(19) United States
(12) Patent Application Publication
(10) Pub. No.: US 2014/0017242 A1
(43) Pub. Date: Jan. 16, 2014

(54) ANTI-BRADYKININ B2 RECEPTOR (BKB2R) MONOCLONAL ANTIBODY

(71) Applicant: Diamedica, Inc., Winnipeg (CA)

(72) Inventors: Mark S. Williams, Plymouth, MN (US); Matthew L. Charles, St. Louis Park, MN (US)

(73) Assignee: DIAMEDICA INC., Winnipeg (CA)

(21) Appl. No.: 13/991,120

(22) PCT Filed: Dec. 1, 2011
(86) PCT No.: PCT/US11/62967

§ 371 Date: Aug. 19, 2013

Related U.S. Application Data
(60) Provisional application No. 61/522,586, filed on Aug. 11, 2011.
(60) Provisional application No. 61/419,609, filed on Dec. 3, 2010.

(51) Int. Cl. C07K 16/28

(52) U.S. Cl. CPC C07K 16/28
USPC 424/135.1; 424/139.1; 424/133.1

(57) ABSTRACT

The present invention relates generally to anti-bradykinin B2 receptor (BKB2R) antibodies and methods for making and using them. In particular, the anti-BKB2R antibodies having the variable region sequences described herein are useful for altering one or more of BKB2R of and/or GSK-3 signaling pathways for the treatment of diseases, disorders and conditions such as cancer, diabetes, cardiovascular disorders and other conditions.

### SEQUENCE LISTING

The Sequence Listing associated with this application is provided in text format in lieu of a paper copy, and is hereby incorporated by reference into the specification. The name of the text file containing the Sequence Listing is 260065_{—}401PC_SEQUENCE_LISTING.txt. The text file is about 73 KB, was created on Dec. 1, 2011, and is being submitted electronically via EFS-Web.

### BACKGROUND

1. Technical Field

The presently disclosed invention embodiments relate generally to anti-bradykinin B2 receptor (BKB2R) antibodies and to methods of making and using such antibodies. In particular, the methods described herein are useful for the treatment of diseases and disorders that are associated with biological signal transduction pathways that are influenced by BKB2R activity, such as diabetes and cancer, and related conditions.

2. Description of the Related Art

There are two generally recognized forms of diabetes. In type 1 diabetes, or insulin-dependent diabetes mellitus (IDDM), patients produce little or no insulin, the hormone which regulates glucose utilization. In type 2 diabetes, or noninsulin dependent diabetes mellitus (NIDDM), patients often have plasma insulin levels that are the same or even elevated compared to nondiabetic subjects; however, these patients have developed a resistance to the insulin stimulating effect on glucose and lipid metabolism in the main insulin-sensitive tissues, which are muscle, liver and adipose tissues, and the plasma insulin levels, while elevated, are insufficient to overcome the pronounced insulin resistance.

Current pharmacological therapies for type 2 DM include injected insulin, and oral agents that are designed to lower blood glucose levels. Currently available oral agents include (i) the sulfonylureas, which act by enhancing the sensitivity of the pancreatic beta cell to glucose, thereby increasing insulin secretion in response to a given glucose load; (ii) the biguanides, which improve glucose disposal rates and inhibit hepatic glucose output; (iii) the thiazolidinediones, which improve peripheral insulin sensitivity through interaction with nuclear peroxisome proliferator-activated receptors (PPAR, see, e.g., Spiegelman, 1998 Diabetes 47:507-514; Schoonjans et al., 1997 Curr. Opin. Lipidol. 8:159-166; Staels et al., 1997 Biochimie 79:95-99), (iv) repaglinide, which enhances insulin secretion through interaction with ATP-dependent potassium channels; and (v) acarbose, which decreases intestinal absorption of carbohydrates. Injectable agents include metformin, alpha-glucosidase blockers, CLP-1 and CLP-1 analogues, and DPP-1V inhibitors. However, the use of these conventional antidiabetic or antihyperglycemic agents can be associated with various adverse effects, and eventually the patients may become resistant to the effects of these agents or the diabetes progresses to a more advanced state wherein the agents are no longer effective.

In the monitoring of the treatment of diabetes mellitus the HbA1c value, the product of a non-enzymatic glycation of the haemoglobin B chain, is of exceptional importance. As its formation depends essentially on the blood sugar level and on the lifetime of erythrocytes, the HbA1c value in the sense of a “blood sugar memory” reflects the average blood sugar level of the preceding 4-12 weeks. Diabetic patients whose HbA1c level has been well controlled over a long time by more intensive diabetes treatment (i.e., <6.5% of the total haemoglobin in the sample) are significantly better protected from diabetic microangiopathy. The available treatments for diabetes can give the diabetic subject an average improvement in HbA1c level by on the order of 1.0-1.5%. This reduction in the HbA1C level is not sufficient in all diabetics to bring them into the desired target range of <7.0%, preferably <6.5% and more preferably <6% HbA1c.

At the cellular level, the degenerative phenotype that may be characteristic of late onset diabetes mellitus includes, for example, impaired insulin secretion, decreased ATP synthesis and increased levels of reactive oxygen species. Studies have shown that type 2 DM may be preceded by or associated with certain related disorders. For example, it is estimated that forty million individuals in the U.S. suffer from impaired glucose tolerance (IGT). Following a glucose load, circulating glucose concentrations in IGT patients rise to higher levels, and return to baseline levels more slowly, than in unaffected individuals. A small percentage of IGT individuals (5-10%) progress to non-insulin dependent diabetes (NIDDM) each year. This form of diabetes mellitus, type 2 DM, is associated with decreased release of insulin by pancreatic beta cells and a decreased end-organ response to insulin. Other symptoms of diabetes mellitus and conditions that precede or are associated with diabetes mellitus include obesity, vascular pathologies, peripheral and sensory neuropathies and blindness.

It is clear that none of the current pharmacological therapies corrects the underlying biochemical defect in type 2 DM. Neither do any of these currently available treatments improve all of the physiological abnormalities in type 2 DM such as impaired insulin secretion, insulin resistance and/or excessive hepatic glucose output. In addition, treatment failures are common with these agents, such that multi-drug therapy is frequently necessary.

The cell surface bradykinin B2 receptor (BKB2R) in mammals (e.g., human BKB2R, SEQ ID NO:71; murine BKB2R, SEQ ID NO:72) mediates kinins and is a G-coupled protein receptor (Leeb-Lundberg et al, 2005 Pharmacol Rev 57:27-77; Belanger et al, 2009 Peptides 30:777-787). BKB2R receptors have high affinity for bradykinin (BK) and kallidin, and are responsible for mediating the majority of known BK physiological effects. BK and other kinins are known to have various organ-protective and cardioprotective effects. Via the BKB2R, BK aids in releasing organ-protecting molecules such as nitric oxide, prostaglandins, and tissue type plasminogen activator. BK also triggers translocation of the glucose transporter GLUT4 from the cytoplasm to the cell surface plasma membrane. Therefore, agonism of BKB2R is thought to have potential therapeutic effects in diabetes and related conditions, and in cardiovascular conditions such as hypertension, hypertrophy, atherosclerosis and ischemic heart disease. BKB2R activation is also thought to be beneficial, insofar as one of its most important effects is the downstream inhibition of glycogen synthase kinase-3 beta (GSK-3(3), a major pharmacological target that has been linked to a wide variety of diseases (Meijer et al, 2004 Trends Pharmacol Sci 25:9, 471-80).

Kallidin, which is an agonist of BKB2R, activates the receptor and in turn triggers the downstream inhibitory phosphorylation (on the serine residue at position number 9) of GSK-36, leading to increased glycogen synthesis (Stambolic et al, 1994 Biochem J 303, 701-704). The activation of the BKB2R receptor also promotes the release of nitric oxide (NO), leading to vasodilation and increased delivery of insulin to tissues; and triggers glucose transporter-4 (GLUT4) translocation to the cell surface, facilitating increased glucose uptake by cells (Kishi et al, 1998 Diabetes 47:4, 550-8).

GSK-3β is located intracellularly, within the cytoplasm, and is thus largely inaccessible to extracellular antibodies. GSK-3β is a constitutively active kinase that regulates multiple signaling pathways (e.g., Wnt pathway, insulin pathway), and GSK-3β also regulates multiple transcription factors via phosphorylation (Doble et al, 2003 J Cell Sci 116: 1175-86). Hence, GSK-3β is regarded as a primary central mediator (“master switch”) of several cellular and developmental functions (e.g., metabolism, cell cycle, cell motility, cytokine expression, and apoptosis). GSK-3β activity is tightly controlled via multiple mechanisms including (i) receptor-mediated signalling which leads to inhibitory phosphorylation of GSK-3 beta, (ii) a requirement in certain cases for “priming phosphorylation” by other kinases of a GSK-3β substrate-binding recognition sequence on GSK-3β target proteins, prior to availability of such substrates for GSK-3β action, (iii) specific GSK-3β intermolecular interactions with a number of defined multi-protein complexes, and (iv) regulated GSK-3β subcellular localization. Given the centrality of GSK-3β to multiple biological processes in cells, a breakdown in regulation of GSK-3β (e.g., in cases of excessive GSK-3β activity with deleterious consequences) has been implicated in a variety of diseases and disorders (Doble et al, 2003 J Cell Sci 116: 1175-86).

Despite recent attention that has been recently focused on GSK-3β, and nomination of GSK-3β by the pharmaceutical industry as a target for drug development, the development of effective GSK-3β inhibitors has been largely unsuccessful, due in part to its central role as a mediator of multiple intracellular pathways without the availability of specific tools that selectively influence desired biological effects. Clearly there is a need for a refined approach to exploit regulation by GSK-3β of particular biological signal transduction in a selective manner, including in clinically relevant contexts. The presently disclosed invention addresses this need, and provides other related advantages.

### BRIEF SUMMARY

According to certain embodiments of the invention described herein, there is provided an isolated antibody, or an antigen-binding fragment thereof, that binds to a human bradykinin B2 receptor (BKB2R), comprising a heavy chain variable region that comprises VHCDR1, VHCDR2 and VHCDR3 amino acid sequences; and a light chain variable region that comprises VLCDR1, VLCDR2 and VLCDR3 amino acid sequences, wherein at least one of: (1) (A) the VHCDR1, VHCDR2 and VHCDR3 amino acid sequences comprise, respectively, the amino acid sequences set forth in (i) SEQ ID NOS:19, 20 and 21, (ii) SEQ ID NOS:22, 23 and 24, or (iii) SEQ ID NOS:25, 26 and 27; and (B) the VLCDR1, VLCDR2 and VLCDR3 amino acid sequences comprise, respectively, the amino acid sequences set forth in (i) SEQ ID NOS:34, 35 and 36, (ii) SEQ ID NOS:37, 38 and 39, or (iii) SEQ ID NOS:40, 41 and 42; or (2) (A) the VHCDR1, VHCDR2 and VHCDR3 amino acid sequences comprise, respectively, the amino acid sequences set forth in (i) SEQ ID NOS:13, 14 and 15, or (ii) SEQ ID NOS:16, 17 and 18; and (B) the VLCDR1, VLCDR2 and VLCDR3 amino acid sequences comprise, respectively, the amino acid sequences set forth in (i) SEQ ID NOS:28, 29 and 30, or (ii) SEQ ID NOS:31, 32 and 33.

In certain further embodiments, the heavy chain variable region comprises the VHCDR1, VHCDR2 and VHCDR3 amino acid sequences set forth in SEQ ID NOS:22, 23 and 24, respectively, and the light chain variable region comprises the VLCDR1, VLCDR2 and VLCDR3 amino acid sequences set forth in SEQ ID NOS:40, 41 and 42, respectively. In certain still further embodiments the heavy chain variable region comprises the amino acid sequence set forth in SEQ ID NO: 6. In certain other embodiments the light chain variable region comprises the amino acid sequence set forth in SEQ ID NO:12. In certain embodiments the light chain variable region comprises the amino acid sequence set forth in any one of SEQ ID NOS:8-12. In certain further embodiments the isolated antibody, or an antigen-binding fragment thereof, comprises a heavy chain variable domain that comprises an amino acid sequence having at least 95% identity to the amino acid sequence set forth in any one of SEQ ID NOS:3-7.

In certain embodiments the heavy chain variable region comprises the amino acid sequence set forth in any one of SEQ ID NOS:3-7. In certain further embodiments the isolated antibody, or an antigen-binding fragment thereof, comprises a light chain variable domain that comprises an amino acid sequence having at least 95% identity to the amino acid sequence set forth in any one of SEQ ID NOS:8-12.

In certain embodiments the heavy chain variable region comprises the VHCDR1, VHCDR2 and VHCDR3 amino acid sequences set forth in SEQ ID NOS:19, 20 and 21, respectively, and the light chain variable region comprises the VLCDR1, VLCDR2 and VLCDR3 amino acid sequences set forth in SEQ ID NOS:37, 38 and 39, respectively. In certain further embodiments the heavy chain variable region comprises the amino acid sequence set forth in SEQ ID NO: 5. In certain other further embodiments the light chain variable region comprises the amino acid sequence set forth in SEQ ID NO:11.

In certain embodiments there is provided an isolated antibody, or an antigen-binding fragment thereof, that binds to a human bradykinin B2 receptor (BKB2R), comprising a heavy chain variable region that comprises the amino acid sequence set forth in SEQ ID NO:1; and a light chain variable region that comprises the VLCDR3 amino acid sequence set forth in SEQ ID NO:2.

In certain embodiments of the above described isolated antibody or antigen-binding fragment thereof, the antibody is humanized. In certain further embodiments the light chain variable domain comprises the amino acid sequence set forth in any one of SEQ ID NOS:8-12. In certain still further embodiments, the isolated antibody, or antigen-binding fragment thereof, comprises a heavy chain variable domain that comprises an amino acid sequence having at least 95% identity to the amino acid sequence set forth in any one of SEQ ID NOS:3-7. In certain embodiments, the isolated antibody or antigen-binding fragment thereof comprises a heavy chain variable domain that comprises the amino acid sequence set forth in any one of SEQ ID NOS:3-7.

In certain embodiments, any of the above described isolated antibodies, or antigen-binding fragments thereof, comprises a human immunoglobulin kappa light chain constant region comprising the amino acid sequence set forth in either SEQ ID NO:77 or SEQ ID NO:81. In certain embodiments, any of the above described isolated antibodies, or antigen-binding fragments thereof, comprises a human immunoglobulin IgG2 heavy chain constant region comprising the amino acid sequence set forth in either SEQ ID NO:75 or SEQ ID NO:79.

In certain embodiments of the above described subject matter, the isolated antibody, or an antigen-binding fragment thereof, comprises either one or both of (a) an immunoglobulin IgG2 heavy chain that comprises the amino acid sequence set forth in any one of SEQ ID NOS:83-87; and (b) an immunoglobulin kappa light chain that comprises the amino acid sequence set forth in any one of SEQ ID NOS:88-92. In certain embodiments, any of the above described isolated antibodies, or antigen-binding fragments thereof, comprises an antibody that is selected from a single chain antibody, a ScFv, a univalent antibody lacking a hinge region, and a minibody. In certain embodiments, any of the above described isolated antibodies, or antigen-binding fragments thereof, comprises a Fab or a Fab′ fragment. In certain embodiments, any of the above described isolated antibodies, or antigen-binding fragments thereof, is a F(ab′)₂ fragment. In certain embodiments, any of the above described isolated antibodies is a whole antibody. In certain embodiments, any of the above described isolated antibodies, or antigen-binding fragments thereof, comprises a human IgG Fc domain.

In certain embodiments there is provided a composition comprising a physiologically acceptable carrier and a therapeutically effective amount of any of the above described isolated antibodies, or antigen-binding fragments thereof.

In certain embodiments there is provided a method for treating a patient with diabetes and having a condition associated with BKB2R activity that is selected from hyperglycemia, hypercholesterolemia, hypertension, cardiovascular disease, retinopathy, nephropathy, neuropathy and insulin resistance, the method comprising administering to the patient the composition comprising a physiologically acceptable carrier and a therapeutically effective amount of any of the above described isolated antibodies, or antigen-binding fragments thereof, and thereby treating the condition associated with BKB2R activity. In certain embodiments there is provided a method for treating a patient with cardiovascular disease, comprising administering to the patient the composition comprising a physiologically acceptable carrier and a therapeutically effective amount of any of the above described isolated antibodies, or antigen-binding fragments thereof, thereby treating the cardiovascular disease. In certain embodiments there is provided a method for treating a patient with hypercholesterolemia, comprising administering to the patient the composition comprising a physiologically acceptable carrier and a therapeutically effective amount of any of the above described isolated antibodies, or antigen-binding fragments thereof, thereby treating the hypercholesterolemia. In certain embodiments there is provided a method for treating a patient with hypertension, comprising administering to the patient the composition comprising a physiologically acceptable carrier and a therapeutically effective amount of any of the above described isolated antibodies, or antigen-binding fragments thereof, thereby treating the hypertension.

In certain embodiments there is provided a method for treating or preventing a cancer that is sensitive to GSK3-β inhibition, comprising administering, to a patient having the cancer, the composition comprising a physiologically acceptable carrier and a therapeutically effective amount of any of the above described isolated antibodies, or antigen-binding fragments thereof, and thereby treating or preventing the cancer. In certain embodiments the cancer is selected from mixed lineage leukemia, esophageal cancer, ovarian cancer, prostate cancer, kidney cancer, colon cancer, liver cancer, stomach cancer, and pancreatic cancer. In certain embodiments there is provided a method of inhibiting the proliferation or survival of a cancer cell, wherein the cancer cell operably expresses a BKB2R protein in a GSK3-B signaling pathway, said method comprising contacting the cancer cells with the composition comprising a physiologically acceptable carrier and a therapeutically effective amount of any of the above described isolated antibodies, or antigen-binding fragments thereof.

In certain embodiments there is provided a method of inhibiting signaling by a GSK3-B signaling pathway in a cell operably expressing a BKB2R protein, comprising contacting the cell with any of the above described antibodies, or an antigen-binding fragment thereof. In certain embodiments there is provided a method for altering at least one of (i) radiation exposure (ii) influenza infection, and (iii) stroke in a BKB2R-expressing cell, comprising contacting the cell with any of the above described antibodies, or an antigen-binding fragment thereof, under conditions and for a time sufficient for specific binding of the antibody to the cell.

These and other aspects and embodiments of the herein described invention will be evident upon reference to the following detailed description and attached drawings. All of the U.S. patents, U.S. patent application publications, U.S. patent applications, foreign patents, foreign patent applications and non-patent publications referred to in this specification and/or listed in the Application Data Sheet are incorporated herein by reference in their entirety, as if each was incorporated individually. Aspects and embodiments of the invention can be modified, if necessary, to employ concepts of the various patents, applications and publications to provide yet further embodiments.

### BRIEF DESCRIPTION OF THE DRAWINGS

FIG. 1 is a bar graph illustrating the induction of GSK-3β inhibition in vivo by anti-BKB2R monoclonal antibodies. The graph shows the level of GSK-3β phosphorylation on serine-9 in 3T3 mouse cells as measured by ELISA, as an indication of GSK-3β inhibition.

FIG. 2 is a bar graph illustrating the induction of GSK-3β inhibition by anti-BKB2R monoclonal antibodies. The graph shows the level of GSK-3β phosphorylation on serine-9 in WI-38 human cells as measured by ELISA, as an indication of GSK-3β inhibition.

FIG. 3 is a graph of acute monoclonal antibody dose response. The graph plots the average mean arterial pressure response for all four indicated anti-BKB2R monoclonal antibody groups following infusion. Data points for each group are presented as mean±Standard Error.

FIG. 4 is a graph depicting the effect of anti-BKB2R monoclonal antibodies on blood pressure one, two and three hours after in vivo administration. The graph plots the mean±SEM for each group (*p<0.05 vs baseline for 5F12G1).

FIG. 5 shows that Tamiflu® reduced influenza replication in A549 cells, as determined by qRT-PCR. The graph shows the increase in relative fluorescence that reflected increasing displacement and cleavage of the Taqman® probe in direct proportion to the amplified portion of the influenza M segment. Samples with lower Tamiflu concentrations increased in fluorescence at an earlier Ct (threshold cycle), indicating a higher viral titer.

FIG. 6 shows an actual and trended plot of the Ct (y-axis) versus the Tamiflu® concentration (x-axis) at a fluorescence threshold of 1500 fluorescence units. Tamiflu® decreased viral titer in a dose dependent manner.

FIG. 7 shows a graph evidencing that anti-BKB2R monoclonal antibody 5F12G1 (“G1”) reduced influenza replication in A549 cells, as determined by qRT-PCR. The graph shows the increase in relative fluorescence that reflected increasing displacement and cleavage of the Taqman® probe in direct proportion to the amplified portion of the influenza M segment. Samples with lower G1 concentrations increased in fluorescence at an earlier Ct (threshold cycle), indicating a higher viral titer.

FIG. 8 shows an actual and trended plot of the Ct (y-axis) versus the anti-BKB2R monoclonal antibody 5F12G1 (“G1”) concentration (x axis) at a fluorescence threshold of 1500 fluorescence units. G1 decreased viral titer in a dose-dependent manner.

FIG. 9 shows the percentage of the control cell viability and the percentage reduction of cytopathic effect (CPE) for the anti-BKB2R monoclonal antibody G1 versus A/Brisbane/59/07 in MDCK cells.

FIG. 10 shows the percentage of the control cell viability and the percentage reduction of CPE for the anti-BKB2R monoclonal antibody G7 versus A/Brisbane/59/07 in MDCK cells.

FIG. 11 shows the percentage of the control cell viability and the percentage reduction of CPE for the anti-BKB2R monoclonal antibody H9 versus A/Brisbane/59/07 in MDCK cells.

FIG. 12 shows the percentage of the control cell viability and the percentage reduction of CPE for the anti-BKB2R monoclonal antibody H3 versus A/Brisbane/59/07 in MDCK cells.

FIG. 13 shows the percentage of the control cell viability and the percentage reduction of CPE for Tamiflu® versus A/Brisbane/59/07 in MDCK cells.

FIG. 14 shows the percentage of the control cell viability and the percentage reduction of CPE for the anti-BKB2R monoclonal antibody G1 versus influenza (CA/07/09) in MDCK cells.

FIG. 15 shows the percentage of the control cell viability and the percentage reduction of CPE for the anti-BKB2R monoclonal antibody G7 versus influenza (CA/07/09) in MDCK cells.

FIG. 16 shows the percentage of the control cell viability and the percentage reduction of CPE for the anti-BKB2R monoclonal antibody H9 versus influenza (CA/07/09) in MDCK cells.

FIG. 17 shows the percentage of the control cell viability and the percentage reduction of CPE for the anti-BKB2R monoclonal antibody H3 versus influenza (CA/07/09) in MDCK cells.

FIG. 18 shows the percentage of the control cell viability and the percentage reduction of CPE for Tamiflu® versus influenza (CA/07/09) in MDCK cells.

FIG. 19 shows BxPC-3 cell viability as a percentage of control when treated with various concentrations of the anti-BKB2R monoclonal antibodies 1F2G7 and 5F12G1.

FIG. 20 shows MV-4-11 cell viability as a percentage of control when treated with various concentrations of the anti-BKB2R monoclonal antibodies 1F2G7 and 5F12G1.

FIG. 21 shows Hep G2 cell viability as a percentage of control when treated with various concentrations of the anti-BKB2R monoclonal antibodies 1F2G7 and 5F12G1.

FIG. 22 shows RS4;11 cell viability as a percentage of control when treated with various concentrations of the anti-BKB2R monoclonal antibodies 1F2G7 and 5F12G1.

FIG. 23 shows HT-29 cell viability as a percentage of control when treated with various concentrations of the anti-BKB2R monoclonal antibodies 1F2G7 and 5F12G1.

FIG. 24 shows NUGC-4 cell viability as a percentage of control when treated with various concentrations of the anti-BKB2R monoclonal antibodies 1F2G7 and 5F12G1.

FIG. 25 shows PC-3 cell viability as a percentage of control when treated with various concentrations of the anti-BKB2R monoclonal antibodies 1F2G7 and 5F12G1.

FIG. 26 shows the glucose infusion rate of monoclonal anti-BKB2R antibody F512G1 in the hyperinsulinemic euglycemic clamps, compared to a vehicle control.

FIG. 27 shows the glucose infusion rate AUC of monoclonal anti-BKB2R antibody F512G1 in the hyperinsulinemic euglycemic clamps, compared to a vehicle control.

FIG. 28A shows the blood glucose levels during an oral glucose tolerance test in Zucker rats treated with various doses of monoclonal antibody 5F12G1.

FIG. 28B shows the area under the curve (AUC) of blood glucose levels during an oral glucose tolerance test in Zucker rats treated with various doses of monoclonal antibody 5F12G1.

FIG. 29A shows the serum insulin levels during an oral glucose tolerance test in Zucker rats treated with various doses of monoclonal antibody 5F12G1.

FIG. 29B shows the area under the curve (AUC) of serum insulin levels during an oral glucose tolerance test in Zucker rats treated with various doses of monoclonal antibody 5F12G1.

FIG. 30A shows the blood glucose levels during an oral glucose tolerance test in DIO mice treated with various doses of monoclonal antibody 5F12G1.

FIG. 30B shows the area under the curve (AUC) of blood glucose levels during an oral glucose tolerance test in DIO mice treated with various doses of monoclonal antibody 5F12G1.

FIG. 31 shows the serum insulin levels during an oral glucose tolerance test in DIO mice treated with various doses of monoclonal antibody 5F12G1.

FIG. 32A shows the blood glucose levels during an oral glucose tolerance test in ZDF fa/fa rats at day 0, and FIG. 32B shows the blood glucose levels during an oral glucose tolerance test on day 21, after treatment with various doses of monoclonal antibody 5F12G1, exenatide, sitagliptin or MG2b-57.

FIG. 33 shows the area under the curve (AUC) of blood glucose levels in ZDF fa/fa rats during an oral glucose tolerance on day 21 after treatment with various doses of 5F12G1, exenatide, sitagliptin or MG2b-57.

FIG. 34A shows the serum insulin levels during an oral glucose tolerance test in ZDF fa/fa rats at day 0, and FIG. 32B shows serum insulin levels during an oral glucose tolerance test in ZDF fa/fa rats on day 21, after treatment with various doses of monoclonal antibody 5F12G1, exenatide, sitagliptin or MG2b-57.

FIG. 35 shows the fasting blood glucose levels in ZDF fa/fa rats from day 0 to day 21 of treatment with various doses of monoclonal antibody 5F12G1, exenatide, sitagliptin or MG2b-57.

FIG. 36 shows the serum cholesterol levels in ZDF fa/fa rats on day 21 after treatment with various doses of 5F12G1, exenatide, sitagliptin or MG2b-57.

FIG. 37 shows the percent glycosylated hemoglobin (HbA1c) levels in ZDF fa/fa rats on day 21 after treatment with various doses of monoclonal antibody 5F12G1, exenatide, sitagliptin or MG2b-57.

FIG. 38 shows the levels of glucose detected in the urine of ZDF fa/fa rats on day 14 after treatment with various doses of 5F12G1, exenatide, sitagliptin or MG2b-57.

FIG. 39A shows the systolic blood pressure in ZDF fa/fa rats at day 0, and FIG. 39B shows the systolic blood pressure on day 21, after treatment with various doses of 5F12G1, exenatide, sitagliptin or MG2b-57.

FIG. 40A shows the diastolic blood pressure in ZDF fa/fa rats at day 0, and FIG. 40B shows the diastolic blood pressure on day 21 after treatment with various doses of 5F12G1, exenatide, sitagliptin or MG2b-57.

FIG. 41A shows the heart rate in ZDF fa/fa rats at day 0, and FIG. 41B shows the heart rate on day 21, after treatment with various doses of 5F12G1, exenatide, sitagliptin or MG2b-57.

FIG. 42 shows the area under the curve (AUC) of glucose infusion rate in ZDF fa/fa rats during an hyperinsulinemic-euglycemic clamp on day 21 after treatment with various doses of 5F12G1, exenatide, sitagliptin or MG2b-57.

FIG. 43 summarizes the area under the curve (AUC) data from an oral glucose tolerance test that monitored blood glucose concentration following single administration of 5F12G1 or humanized anti-BKB2R monoclonal antibodies, after oral administration of glucose in ZDF fa/fa rats, as compared to a vehicle control.

### BRIEF DESCRIPTION OF THE SEQUENCES

SEQ ID NO:1 is the amino acid sequence of the murine heavy chain variable region of the 5F12G1 anti-BKB2R antibody.

SEQ ID NO:2 is the amino acid sequence of the murine light chain variable region of the 5F12G1 anti-BKB2R antibody.

SEQ ID NO:3 is the amino acid sequence of the H1 heavy chain variable region of the humanized anti-BKB2R antibody.

SEQ ID NO:4 is the amino acid sequence of the H2 heavy chain variable region of the humanized anti-BKB2R antibody.

SEQ ID NO:5 is the amino acid sequence of the H37 heavy chain variable region of the humanized anti-BKB2R antibody.

SEQ ID NO:6 is the amino acid sequence of the H38 heavy chain variable region of the humanized anti-BKB2R antibody.

SEQ ID NO:7 is the amino acid sequence of the H39 heavy chain variable region of the humanized anti-BKB2R antibody.

SEQ ID NO:8 is the amino acid sequence of the L1 light chain variable region of the humanized anti-BKB2R antibody.

SEQ ID NO:9 is the amino acid sequence of the L2 light chain variable region of the humanized anti-BKB2R antibody.

SEQ ID NO:10 is the amino acid sequence of the L37 light chain variable region of the humanized anti-BKB2R antibody.

SEQ ID NO:11 is the amino acid sequence of the L38 light chain variable region of the humanized anti-BKB2R antibody.

SEQ ID NO:12 is the amino acid sequence of the L39 light chain variable region of the humanized anti-BKB2R antibody.

SEQ ID NO:13 is the amino acid sequence of the H1 VHCDR1 of the humanized anti-BKB2R antibody.

SEQ ID NO:14 is the amino acid sequence of the H1 VHCDR2 of the humanized anti-BKB2R antibody.

SEQ ID NO:15 is the amino acid sequence of the H1 VHCDR3 of the humanized anti-BKB2R antibody.

SEQ ID NO:16 is the amino acid sequence of the H2 VHCDR1 of the humanized anti-BKB2R antibody.

SEQ ID NO:17 is the amino acid sequence of the H2 VHCDR2 of the humanized anti-BKB2R antibody.

SEQ ID NO:18 is the amino acid sequence of the H2 VHCDR3 of the humanized anti-BKB2R antibody.

SEQ ID NO:19 is the amino acid sequence of the H37 VHCDR1 of the humanized anti-BKB2R antibody.

SEQ ID NO:20 is the amino acid sequence of the H37 VHCDR2 of the humanized anti-BKB2R antibody.

SEQ ID NO:21 is the amino acid sequence of the H37 VHCDR3 of the humanized anti-BKB2R antibody.

SEQ ID NO:22 is the amino acid sequence of the H38 VHCDR1 of the humanized anti-BKB2R antibody.

SEQ ID NO:23 is the amino acid sequence of the H38 VHCDR2 of the humanized anti-BKB2R antibody.

SEQ ID NO:24 is the amino acid sequence of the H38 VHCDR3 of the humanized anti-BKB2R antibody.

SEQ ID NO:25 is the amino acid sequence of the H39 VHCDR1 of the humanized anti-BKB2R antibody.

SEQ ID NO:26 is the amino acid sequence of the H39 VHCDR2 of the humanized anti-BKB2R antibody.

SEQ ID NO:27 is the amino acid sequence of the H39 VHCDR3 of the humanized anti-BKB2R antibody.

SEQ ID NO:28 is the amino acid sequence of the L1 VLCDR1 of the humanized anti-BKB2R antibody.

SEQ ID NO:29 is the amino acid sequence of the L1 VLCDR2 of the humanized anti-BKB2R antibody.

SEQ ID NO:30 is the amino acid sequence of the L1 VLCDR3 of the humanized anti-BKB2R antibody.

SEQ ID NO:31 is the amino acid sequence of the L2 VLCDR1 of the humanized anti-BKB2R antibody.

SEQ ID NO:32 is the amino acid sequence of the L2 VLCDR2 of the humanized anti-BKB2R antibody.

SEQ ID NO:33 is the amino acid sequence of the L2 VLCDR3 of the humanized anti-BKB2R antibody.

SEQ ID NO:34 is the amino acid sequence of the L37 VLCDR1 of the humanized anti-BKB2R antibody.

SEQ ID NO:35 is the amino acid sequence of the L37 VLCDR2 of the humanized anti-BKB2R antibody.

SEQ ID NO:36 is the amino acid sequence of the L37 VLCDR3 of the humanized anti-BKB2R antibody.

SEQ ID NO:37 is the amino acid sequence of the L38 VLCDR1 of the humanized anti-BKB2R antibody.

SEQ ID NO:38 is the amino acid sequence of the L38 VLCDR2 of the humanized anti-BKB2R antibody.

SEQ ID NO:39 is the amino acid sequence of the L38 VLCDR3 of the humanized anti-BKB2R antibody.

SEQ ID NO:40 is the amino acid sequence of the L39 VLCDR1 of the humanized anti-BKB2R antibody.

SEQ ID NO:41 is the amino acid sequence of the L39 VLCDR2 of the humanized anti-BKB2R antibody.

SEQ ID NO:42 is the amino acid sequence of the L39 VLCDR3 of the humanized anti-BKB2R antibody.

SEQ ID NO:43 is the amino acid sequence of the VHCDR1 of the murine 5F12G1 anti-BKB2R antibody.

SEQ ID NO:44 is the amino acid sequence of the VHCDR2 of the murine 5F12G1 anti-BKB2R antibody.

SEQ ID NO:45 is the amino acid sequence of the VHCDR3 of the murine 5F12G1 anti-BKB2R antibody.

SEQ ID NO:46 is the amino acid sequence of the VLCDR1 of the murine 5F12G1 anti-BKB2R antibody.

SEQ ID NO:47 is the amino acid sequence of the VLCDR2 of the murine 5F12G1 anti-BKB2R antibody.

SEQ ID NO:48 is the amino acid sequence of the VLCDR3 of the murine 5F12G1 anti-BKB2R antibody.

SEQ ID NO:49 is the polynucleotide encoding the amino acid sequence of SEQ ID NO:1, i.e., encoding the murine heavy chain variable region for the 5F12G1 anti-BKB2R antibody.

SEQ ID NO:50 is the polynucleotide encoding the amino acid sequence of SEQ ID NO:2, i.e., encoding the murine light chain variable region for the 5F12G1 antibody.

SEQ ID NO:51 is the polynucleotide encoding the amino acid sequence of SEQ ID NO: 3, i.e., encoding the H1 humanized heavy chain variable region for the anti-BKB2R antibody.

SEQ ID NO:52 is the polynucleotide encoding the amino acid sequence of SEQ ID NO: 4, i.e., encoding the H2 humanized heavy chain variable region for the anti-BKB2R antibody.

SEQ ID NO:53 is the polynucleotide encoding the amino acid sequence of SEQ ID NO: 5, i.e., encoding the H37 humanized heavy chain variable region for the anti-BKB2R antibody.

SEQ ID NO:54 is the polynucleotide encoding the amino acid sequence of SEQ ID NO: 6, i.e., encoding the H38 humanized heavy chain variable region for the anti-BKB2R antibody.

SEQ ID NO:55 is the polynucleotide encoding the amino acid sequence of SEQ ID NO: 7, i.e., encoding the H39 humanized heavy chain variable region for the anti-BKB2R antibody.

SEQ ID NO:56 is the polynucleotide encoding the amino acid sequence of SEQ ID NO: 8, i.e., encoding the L1 humanized light chain variable region for the anti-BKB2R antibody.

SEQ ID NO:57 is the polynucleotide encoding the amino acid sequence of SEQ ID NO: 9, i.e., encoding the L2 humanized light chain variable region for the anti-BKB2R antibody.

SEQ ID NO:58 is the polynucleotide encoding the amino acid sequence of SEQ ID NO: 10, i.e., encoding the L37 humanized light chain variable region for the anti-BKB2R antibody.

SEQ ID NO:59 is the polynucleotide encoding the amino acid sequence of SEQ ID NO: 11, i.e., encoding the L38 humanized light chain variable region for the anti-BKB2R antibody.

SEQ ID NO:60 is the polynucleotide encoding the amino acid sequence of SEQ ID NO: 12, i.e., encoding the L39 humanized light chain variable region for the anti-BKB2R antibody.

SEQ ID NOS:61-68 are sequences of oligonucleotide RACE primers.

SEQ ID NOS:69-70 are sequences of oligonucleotide sequencing primers.

SEQ ID NO:71 shows a human BKB2R amino acid sequence.

SEQ ID NO:72 shows a mouse BKB2R amino acid sequence.

SEQ ID NO:73 shows the amino acid sequence of an immunogenic human BKB2R peptide fragment.

SEQ ID NO:74 shows the amino acid sequence of an immunogenic mouse BKB2R peptide fragment.

SEQ ID NO:75 is the amino acid sequence of human immunoglobulin IgG2 heavy chain constant region.

SEQ ID NO:76 is the sequence of the polynucleotide encoding the amino acid sequence of SEQ ID NO:75.

SEQ ID NO:77 is the amino acid sequence of human immunoglobulin kappa light chain constant region.

SEQ ID NO:78 is the sequence of the polynucleotide encoding the amino acid sequence of SEQ ID NO:77.

SEQ ID NO:79 is the amino acid sequence of human immunoglobulin IgG2 heavy chain constant region.

SEQ ID NO:80 is the sequence of the polynucleotide encoding the amino acid sequence of SEQ ID NO:79.

SEQ ID NO:81 is the amino acid sequence of human immunoglobulin kappa light chain constant region.

SEQ ID NO:82 is the sequence of the polynucleotide encoding the amino acid sequence of SEQ ID NO:81.

SEQ ID NO:83 is the amino acid sequence of humanized H1 heavy chain, including the human IgG2 constant region.

SEQ ID NO:84 is the amino acid sequence of humanized H2 heavy chain, including the human IgG2 constant region.

SEQ ID NO:85 is the amino acid sequence of humanized H37 heavy chain, including the human IgG2 constant region.

SEQ ID NO:86 is the amino acid sequence of humanized H38 heavy chain, including the human IgG2 constant region.

SEQ ID NO:87 is the amino acid sequence of humanized H39 heavy chain, including the human IgG2 constant region.

SEQ ID NO:88 is the amino acid sequence of humanized L1 light chain, including the human Ig kappa constant region.

SEQ ID NO:89 is the amino acid sequence of humanized L2 light chain, including the human Ig kappa constant region.

SEQ ID NO:90 is the amino acid sequence of humanized L37 light chain, including the human Ig kappa constant region.

SEQ ID NO:91 is the amino acid sequence of humanized L38 light chain, including the human Ig kappa constant region.

SEQ ID NO:92 is the amino acid sequence of humanized L39 light chain, including the human Ig kappa constant region.

### DETAILED DESCRIPTION

According to certain invention embodiments disclosed herein, there are provided compositions and methods that relate to specific anti-BKB2R monoclonal antibodies, and in particular to humanized anti-BKB2R antibodies having the VHCDR1, VHCDR2, and VHCDR3 sequences and/or the VLCDR1, VLCDR2, and VLCDR3 sequences and/or the VH and/or VL sequences, as described herein. As also described herein, the presently disclosed anti-BKB2R antibodies unexpectedly exhibited agonist activity toward the BKB2R when the antibodies were contacted with BKB2R-expressing cells, and surprisingly resulted in inhibition of GSK-313.

The herein described anti-BKB2R antibodies will find uses in a large number of contexts where intervention and alteration (e.g., a statistically significant increase or decrease, such as in detectable activity level) of BKB2R activity and/or of a biological signalling pathway to which BKB2R activity contributes, may be desirable. For instance, a number of clinically defined conditions appear, according to non-limiting theory, to result from excessive GSK-3β activity, such that the GSK-3β-inhibitory properties that were unexpectedly exhibited by the presently described anti-BKB2R antibodies may be beneficially exploited. Hence, also provided herein are compositions and methods for treating a condition associated with BKB2R activity, which may include but need not be limited to diabetes and/or accompanying risks of cardiovascular disorders, retinopathy, neuropathy or nephropathy, cancer, cardiovascular diseases and a number of related conditions, including high blood pressure, excessive blood glucose concentrations, elevated serum cholesterol concentrations, viral infections, stroke, radiation exposure, or other disease.

The BKB2R represents an initiation point of a known, endogenous cell signaling pathway (PI3K/Akt) which leads to the inhibition of GSK-3β via Ser⁹ phosphorylation. This pathway is utilized endogenously to help regulate blood glucose levels and likely in the process of neurogenesis as well, when the enzyme tissue kallikrein 1 (KLK1) cleaves kininogens to liberate kinins (bradykinin and kallidin (Lys-bradykinin)) that activate the BKB2R receptor. Normally, KLK-1 generates kallidin, a short lived (˜30 seconds in vivo) but potent BKB2 receptor agonist (Kd˜0.89 nM). Triggering the BKB2R G protein coupled receptor by kallidin binding induces downstream signalling events via the PI3K/Akt pathway, leading to the phosphorylation and deactivation of GSK-3β on serine-9. Inhibition of GSK-3β in turn can increase glycogen synthesis, and can also decrease Tau phosphorylation, apoptosis and inflammation. Without wishing to be bound by theory, it is believed that the anti-BKB2R antibodies of certain herein described embodiments of the present invention mimic this pathway by binding to a very specific protein sequence-defined structure on the BKB2 receptor, which leads to BKB2R activation and eventual downstream inhibition of GSK-3β. Further according to non-limiting theory, it is believed that the present monoclonal antibodies specifically target an extracellularly disposed epitope on the BKB2R receptor, such that the antibodies act agonistically. By such specificity, the herein described anti-BKB2R antibodies negate the possibility of “off target” binding that has been previously seen with other GSK-3β inhibitors, beneficially reducing the risk of associated side effects that result from a less specific mechanism of action by the prior inhibitors.

Conditions associated with BKB2R activity include a number of diseases and disorders in which improperly regulated GSK-3β activity has been implicated. Non-limiting illustrative examples include:

(a) radiation exposure—inhibition of GSK-3β in some circumstances can prevent apoptosis via the Bax signalling pathway, a p53-dependent pathway that induces apoptosis, and thus could prevent the loss of bone marrow cells and possibly gastrointestinal mucosal tissue following exposure to harmful levels of whole body radiation. Kallikrein-1 (KLK-1) has been studied as a treatment for radiation exposure although it is not known if the reported effect of KLK-1 on radiation survival is mediated though kallidin action on the BKB2R receptor, or by the activation of growth factors, or a combination of both;

(b) type II diabetes and hypertension—one of the major co-pathologies of type 2 diabetes is hypertension, which can retard the delivery of insulin to tissues but can be lowered via BKB2R receptor activation;

(c) cancer—mixed lineage leukemia cells (MLL) are susceptible to GSK-3β inhibition. This relationship is somewhat counterintuitive as GSK-3β typically activates apoptotic pathways. This mechanism does not involve antibody dependent cell cytotoxicity (ADCC) and does not require a unique cancer specific biomarker (the BKB2 receptor is ubiquitously expressed in cells). Instead, cell death occurs in only those cells sensitive to GSK-3β inhibition. GSK-3β has also been suggested as a potential downstream target in a number of different cancers, such as esophageal, ovarian, prostate, kidney, colon, liver, stomach, and pancreatic cancers;

(d) myocardial infarction and stroke—KLK-1 is known to protect and improve cardiac recovery following ischemia. These effects have been blocked in preclinical studies through the use of BK B2 receptor antagonists (e.g., HOE 140); and

(e) influenza-GSK-3β has been confirmed to be a factor necessary for viral entry into a host cell in Influenza A RNA viruses. Inhibition or blockade of GSK-3β would stop replication and hence attenuate infection.

Embodiments of the present invention thus relate to antibodies that bind to BKB2R, a widely expressed cell surface, G protein-coupled receptor protein (e.g., SEQ ID NO:71), to methods of making such antibodies, and to methods of using such antibodies to alter (e.g., increase or decrease in a statistically significant manner) BKB2R-associated signaling pathway events in BKB2R-expressing cells, including methods that result in inhibition of GSK-3β. The methods described herein are useful for the treatment of conditions associated with BKB2R activity, such as diabetes, cancer and other diseases, disorders, and conditions. Amino acid sequences of illustrative anti-BKB2R antibodies including humanized antibodies, or antigen-binding fragments thereof, or complementarity determining regions (CDRs) thereof, are set forth in SEQ ID NOs:1-48, 75, 77, 79, 81, 83-92, and are encoded by the polynucleotide sequences set forth in SEQ ID NOs:49-60, 76, 78, 80, 82.

In certain embodiments and according to non-limiting theory, the herein described anti-BKB2R antibodies may be contacted with BKB2R-expressing cells, including cells in vivo or ex vivo or isolated cells in vitro, to induce or activate a BKB2R-associated signaling pathway, including in certain embodiments to inhibit GSK-3β. An “isolated” cell is one that has been removed from the natural environment in which it originally occurred, or progeny of such a cell that have been maintained, propagated or generated in vitro.

Accordingly, in certain embodiments the present invention provides a method for altering activity of a BKB2R pathway, comprising contacting a BKB2R-expressing cell with an anti-BKB2R antibody as described herein, under conditions and for a time sufficient for specific binding of the antibody to the cell, wherein a level of activity of a BKB2R pathway is altered (e.g., increased or decreased in a statistically significant manner, and in certain preferred embodiments increased) relative to the level of BKB2R pathway activity that is present in a cell that has not been contacted with the anti-BKB2R antibody.

There are thus expressly contemplated, according to certain of the herein described embodiments, methods by which these and/or related systems may be used to determine or effect the activation or induction by an anti-BKB2R antibody of a BKB2R or a BKB2R-associated signaling pathway, or to determine or effect inhibition by an anti-BKB2R antibody of GSK-3β in a BKB2R-expressing cell.

Criteria for determining activity of a BKB2R-associated signaling pathway are described herein and known in the art and will be appreciated by those skilled in the art. Pathways for biological signal transduction, including those associated with cell division, cell survival, apoptosis, proliferation and differentiation, may in certain instances be referred to as “biological signal transduction pathways,” or “inducible signaling pathways” and may include transient or stable associations or interactions among cellular and extracellular molecular components that are involved in the control of these and similar processes in cells. Depending on the particular pathway(s) of interest, one or more appropriate parameters for determining induction of such pathway(s) may be selected based on art-accepted criteria.

For example, for signaling pathways associated with cellular replication or proliferation, a variety of well known methodologies are available for quantifying replication or proliferation, including, for example, incorporation by proliferating cells of tritiated thymidine into cellular DNA, monitoring of detectable (e.g., fluorimetric or colorimetric) indicators of cellular respiratory activity (for example, conversion of the tetrazolium salts (yellow) 3-(4,5-dimethylthiazol-2-yl)-2,5-diphenyltetrazolium bromide (MTT) or 3-(4,5-dimethylthiazol-2-yl)-5-(3-carboxymethoxyphenyl)-2-(4-sulphophenyl)-2H-tetrazolium (MTS) to formazan dyes (purple) in metabolically active cells), or cell counting, or the like.

Similarly, in the cell biology arts, multiple techniques are known for assessing cell survival by any of a number of known methodologies including viability determination by microscopic, biochemical, spectrophotometric, spectroscopic, light-scattering, cytometric including flow cytometric and cytofluorimetric, or other techniques (e.g., vital dyes such as Trypan Blue, DNA-binding fluorophores such as propidium iodide, metabolic indicators, etc.) and for determining apoptosis (for example, annexin V binding, DNA fragmentation assays, caspase activation, marker analysis, e.g., poly(ADP-ribose) polymerase (PARP), etc.).

Other signaling pathways will be associated with particular cellular phenotypes, for example specific induction of gene expression (e.g., detectable as transcription or translation products, or by bioassays of such products, or as nuclear localization of cytoplasmic factors), altered (e.g., statistically significant increases or decreases) levels of intracellular mediators (e.g., activated kinases or phosphatases, altered levels of cyclic nucleotides or of physiologically active ionic species, altered levels of the degree of phosphorylation of one or more specific phosphorylation substrates, etc.), altered cell cycle profiles, or altered cellular morphology, and the like, such that cellular responsiveness to a particular stimulus as provided herein can be readily identified to determine whether a particular cell is undergoing or has undergone a BKB2R-mediated or a GSK-3β-mediated or other defined signaling pathway-mediated event (e.g., calcium flux assays in BKB2R-expressing cells such as a CHO BKB2R-transfected cell line, assays of GSK-3β phosphorylation such as serine-9 phosphorylation or inhibition of GSK-3β activity, ELISA determination of GSK-3β, GSK-3β binding assays, etc.).

In certain embodiments where it is desirable to determine whether or not a subject or biological source falls within clinical parameters indicative of type 2 diabetes mellitus, signs and symptoms of type 2 diabetes that are accepted by those skilled in the art may be used to so designate a subject or biological source, for example clinical signs referred to in Gavin et al. (Diabetes Care 22(suppl. 1):S5-S19, 1999, American Diabetes Association Expert Committee on the Diagnosis and Classification of Diabetes Mellitus) and references cited therein, or other means known in the art for diagnosing type 2 diabetes.

In diabetes and certain other metabolic diseases or disorders, one or more biochemical processes, which may be either anabolic or catabolic (e.g., build-up or breakdown of substances, respectively), are altered (e.g., increased or decreased in a statistically significant manner) or modulated (e.g., up- or down-regulated to a statistically significant degree) relative to the levels at which they occur in a disease-free or normal subject such as an appropriate control individual. The alteration may result from an increase or decrease in a substrate, enzyme, cofactor, or any other component in any biochemical reaction involved in a particular process. An extensive set of altered indicators of mitochondrial function, for example, has been described for use in determining the presence of, and characterizing, diabetes (see, e.g., U.S. Pat. No. 6,140,067).

BKB2R-related signaling pathway components may include components in the signal transduction pathway induced by insulin and may, for example, be evaluated by determining the level of tyrosine phosphorylation of insulin receptor beta (IR-β) and/or of the downstream signaling molecule PKB/Akt and/or of any other downstream polypeptide that may be a component of a particular signal transduction pathway as provided herein. Conditions associated with BKB2R activity may also include disorders, such as JNK-associated disorders (e.g., cancer, cardiac hypertrophy, ischemia, diabetes, hyperglycemia-induced apoptosis, inflammation, neurodegenerative disorders), and other disorders associated with different signal transduction pathways, for instance, cancer, autoimmunity, cellular proliferative disorders, neurodegenerative disorders, and infectious diseases (see, e.g., Fukada et al., 2001 J. Biol. Chem. 276:25512; Tonks et al., 2001 Curr. Opin. Cell Biol. 13:182; Salmeen et al., 2000 Mol. Cell. 6:1401; Hu et al., J. Neurochem. 85:432-42 (2003); and references cited therein).

The presence of a malignant condition in a subject refers to the presence of dysplastic, cancerous and/or transformed cells in the subject, including, for example neoplastic, tumor, non-contact inhibited or oncogenically transformed cells, or the like (e.g., carcinomas such as adenocarcinoma, squamous cell carcinoma, small cell carcinoma, oat cell carcinoma, etc., sarcomas such as chondrosarcoma, osteosarcoma, etc.) which are known to the art and for which criteria for diagnosis and classification are established (e.g., Hanahan and Weinberg, 2011 Cell 144:646; Hanahan and Weinberg 2000 Cell 100:57; Cavallo et al., 2011 Canc. Immunol. Immunother. 60:319; Kyrigideis et al., 2010 J. Carcinog. 9:3) In preferred embodiments contemplated by the present invention, for example, such cancer cells may be cells of mixed lineage leukemia, esophageal cancer, ovarian cancer, prostate cancer, kidney cancer, colon cancer, liver cancer, stomach cancer, and pancreatic cancer.

### Antibodies and Antigen-Binding Fragments Thereof

An “antibody” is an immunoglobulin molecule capable of specific binding to a target, such as a carbohydrate, polynucleotide, lipid, polypeptide, etc., through at least one epitope recognition site, located in the variable region (also referred to herein as the variable domain) of the immunoglobulin molecule. As used herein, the term “antibody” encompasses not only intact polyclonal or monoclonal antibodies, but also fragments thereof (such as a single variable region antibody (dAb), or other known antibody fragments such as Fab, Fab′, F(ab′)₂, Fv and the like, single chain (ScFv), synthetic variants thereof, naturally occurring variants, fusion proteins comprising an antibody portion with an antigen-binding fragment of the required specificity, humanized antibodies, chimeric antibodies, and any other engineered or modified configuration of the immunoglobulin molecule that comprises an antigen-binding site or fragment (epitope recognition site) of the required specificity. “Diabodies”, multivalent or multispecific fragments constructed by gene fusion (WO94/13804; Holliger et al, Proc. Natl. Acad. Sci. USA 90 6444-6448, 1993) are also a particular form of antibody contemplated herein. Minibodies comprising a scFv joined to a CH3 domain are also included herein (Hu et al, Cancer Res., 56, 3055-3061, 1996; see also e.g., Ward et al., Nature 341, 544-546 (1989); Bird et al, Science 242, 423-426, 1988; Huston et al, PNAS USA, 85, 5879-5883, 1988; PCT/US92/09965; WO94/13804; Holliger et al., Proc. Natl. Acad. Sci. USA 90 6444-6448, 1993; Reiter et al., Nature Biotech 14, 1239-1245, 1996; Hu et al, Cancer Res. 56, 3055-3061, 1996). Nanobodies and maxibodies are also contemplated (see, e.g., U.S. Pat. No. 6,765,087; U.S. Pat. No. 6,838,254; WO 06/079372; WO 2010/037402).

The term “antigen-binding fragment” as used herein refers to a polypeptide fragment that contains at least one CDR of an immunoglobulin heavy and/or light chain that binds to the antigen of interest, which antigen in particularly preferred embodiments described herein is the BKB2R receptor. In this regard, an antigen-binding fragment of the herein described antibodies may comprise one, two, three, four, five or all six CDRs of a VH and/or VL sequence set forth herein from antibodies that bind BKB2R. An antigen-binding fragment of the herein described BKB2R-specific antibodies is capable of binding to BKB2R. In certain embodiments, binding of an antigen-binding fragment prevents or inhibits binding of BKB2R ligand(s) (e.g., bradykinin (BK), kallidin (Lys-bradykinin) to the BKB2R receptor, interrupting the biological response that would otherwise result from ligand binding to the receptor. In certain embodiments, the antigen-binding fragment binds specifically to and/or inhibits or modulates the biological activity of human BKB2R.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes.

The term “epitope” includes any determinant, preferably a polypeptide determinant, that is capable of specific binding to an immunoglobulin or T-cell receptor. An epitope is a region of an antigen that is bound by an antibody. In certain embodiments, epitope determinants include chemically active surface groupings of molecules such as amino acids, sugar side chains, phosphoryl or sulfonyl, and may in certain embodiments have specific three-dimensional structural characteristics, and/or specific charge characteristics. In certain embodiments, an antibody is said to specifically bind an antigen when it preferentially recognizes its target antigen in a complex mixture of proteins and/or macromolecules. An antibody may according to certain embodiments be said to bind an antigen specifically when the equilibrium dissociation constant for antibody-antigen binding is less than or equal to 10⁻⁶M, or less than or equal to 10⁻⁷ M, or less than or equal to 10⁻⁸ M. In some embodiments, the equilibrium dissociation constant may be less than or equal to 10⁻⁹ M or less than or equal to 10⁻¹⁹ M.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment which comprises both antigen-binding sites. An Fv fragment for use according to certain embodiments of the present invention can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule (Inbar et al. (1972) Proc. Nat. Acad. Sci. USA 69:2659-2662; Hochman et al. (1976) Biochem 15:2706-2710; and Ehrlich et al. (1980) Biochem 19:4091-4096).

In certain embodiments, single chain Fv or scFV antibodies are contemplated. For example, Kappa bodies (III et al., Prot. Eng. 10:949-57 (1997); minibodies (Martin et al., EMBO J. 13:5305-9 (1994); diabodies (Holliger et al., PNAS 90:6444-8 (1993)); or Janusins (Traunecker et al., EMBO J. 10:3655-59 (1991) and Traunecker et al. Int. J. Cancer Suppl. 7:51-52 (1992)), may be prepared using standard molecular biology techniques following the teachings of the present application with regard to selecting antibodies having the desired specificity. In still other embodiments, bispecific or chimeric antibodies may be made that encompass the ligands of the present disclosure. For example, a chimeric antibody may comprise CDRs and framework regions from different antibodies, while bispecific antibodies may be generated that bind specifically to BKB2R through one binding domain and to a second molecule through a second binding domain. These antibodies may be produced through recombinant molecular biological techniques or may be physically conjugated together.

A single chain Fv (sFv) polypeptide is a covalently linked V_{H}::V_{L} heterodimer which is expressed from a gene fusion including V_{H}- and V_{L}-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an sFv molecule which will fold into a three-dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

A dAb fragment of an antibody consists of a VH domain (Ward et al., Nature 341, 544-546 (1989)).

In certain embodiments, an antibody as herein disclosed (e.g., an BKB2R-specific antibody) is in the form of a diabody. Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site; antigen binding sites are formed by the association of the first domain of one polypeptide within the multimer with the second domain of another polypeptide within the multimer (WO94/13804).

Where bispecific antibodies are to be used, these may be conventional bispecific antibodies, which can be manufactured in a variety of ways (Holliger and Winter, Current Opinion Biotechnol. 4, 446-449 (1993)), e.g. prepared chemically or from hybrid hybridomas, or may be any of the bispecific antibody fragments mentioned above. Diabodies and scFv can be constructed without an Fc region, using only variable regions, potentially reducing the likelihood or severity of an elicited immune response, such as an anti-idiotypic reaction, in a subject receiving an administration of such antibodies.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knobs-into-holes engineering (Ridgeway et al, Protein Eng., 9, 616-621, 1996).

In certain embodiments, the antibodies described herein may be provided in the form of a UniBody®. A UniBody® is an IgG4 antibody with the hinge region removed (see GenMab Utrecht, The Netherlands; see also, e.g., US/2009/0226421). This proprietary antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells. For certain cancer cell surface antigens, this univalent binding may not stimulate the cancer cells to grow as may be seen using bivalent antibodies having the same antigen specificity, and hence UniBody® technology may afford treatment options for some types of cancer that may be refractory to treatment with conventional antibodies. The UniBody® is about half the size of a regular IgG4 antibody. This small size can be a great benefit when treating some forms of cancer, allowing for better distribution of the molecule over larger solid tumors and potentially increasing efficacy.

In certain embodiments, the antibodies of the present disclosure may take the form of a nanobody. Nanobodies are encoded by single genes and are efficiently produced in almost all prokaryotic and eukaryotic hosts, e.g., E. coli (see e.g. U.S. Pat. No. 6,765,087), molds (for example Aspergillus or Trichoderma) and yeast (for example Saccharomyces, Kluyvermyces, Hansenula or Pichia (see e.g. U.S. Pat. No. 6,838,254)). The production process is scalable and multi-kilogram quantities of nanobodies have been produced. Nanobodies may be formulated as a ready-to-use solution having a long shelf life. The Nanoclone™ method (see, e.g., WO 06/079372) is a proprietary method for generating Nanobodies™ against a desired target, based on automated high-throughput selection of B-cells.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

The structures and locations of immunoglobulin variable regions may be determined by reference to Kabat, E. A. et al, Sequences of Proteins of Immunological Interest, 4th Edition, US Department of Health and Human Services, 1987, and updates thereof, now available on the Internet (immuno.bme.nwu.edu).

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)₂, Fv), single chain (ScFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals, etc.). The term includes whole immunoglobulins as well as the fragments etc. described above.

“Humanized” antibodies refer to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable regions fused onto constant domains or only the CDRs grafted onto appropriate framework regions in the variable domains. Epitope binding sites may be wild type or may be modified by one or more amino acid substitutions. This chimeric structure eliminates the constant region of non-human origin as an immunogen in human individuals, but the possibility of an immune response to the foreign variable region remains (LoBuglio et al., (1989) Proc Natl Acad Sci USA 86:4220-4224; Queen et al., PNAS (1988) 86:10029-10033; Riechmann et al., Nature (1988) 332:323-327). Illustrative humanized antibodies according to certain embodiments of the present invention comprise the humanized sequences provided in SEQ ID NOs:3-12 and 83-92.

Another approach focuses not only on providing human-derived constant regions, but also on modifying the variable regions as well so as to reshape them as closely as possible to human form. As also noted above, it is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato et al., (1993) Cancer Res 53:851-856; Riechmann et al., (1988) Nature 332:323-327; Verhoeyen et al., (1988) Science 239:1534-1536; Kettleborough et al., (1991) Protein Engineering 4:773-3783; Maeda et al., (1991) Human Antibodies Hybridoma 2:124-134; Gorman et al., (1991) Proc Natl Acad Sci USA 88:4181-4185; Tempest et al., (1991) Bio/Technology 9:266-271; Co et al., (1991) Proc Natl Acad Sci USA 88:2869-2873; Carter et al., (1992) Proc Natl Acad Sci USA 89:4285-4289; and Co et al., (1992) J Immunol 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies of the present disclosure may be chimeric antibodies. In this regard, a chimeric antibody is comprised of an antigen-binding fragment of an anti-BKB2R antibody operably linked or otherwise fused to a heterologous Fc portion of a different antibody. In certain embodiments, the heterologous Fc domain is of human origin. In other embodiments, the heterologous Fc domain may be from a different Ig class than the parent antibody, including IgA (including subclasses IgA1 and IgA2), IgD, IgE, IgG (including subclasses IgG1, IgG2, IgG3, and IgG4), and IgM. In certain embodiments, the heterologous Fc domain may be comprised of CH2 and CH3 domains from one or more of the different Ig classes. As noted above with regard to humanized antibodies, the anti-BKB2R antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

In certain embodiments, a BKB2R-binding antibody comprises one or more of the CDRs of the antibodies described herein. In this regard, it has been shown in some cases that the transfer of only the VHCDR3 of an antibody can be done while still retaining desired specific binding (Barbas et al., PNAS (1995) 92: 2529-2533). See also, McLane et al., PNAS (1995) 92:5214-5218, Barbas et al., J. Am. Chem. Soc. (1994) 116:2161-2162.

Marks et al (Bio/Technology, 1992, 10:779-783) describe methods of producing repertoires of antibody variable domains in which consensus primers directed at or adjacent to the 5′ end of the variable domain area are used in conjunction with consensus primers to the third framework region of human VH genes, to provide a repertoire of VH variable domains lacking a CDR3. Marks et al further describe how this repertoire may be combined with a CDR3 of a particular antibody. Using analogous techniques, the CDR3-derived sequences of the presently described antibodies may be shuffled with repertoires of VH or VL domains lacking a CDR3, and the shuffled complete VH or VL domains combined with a cognate VL or VH domain to provide an antibody or antigen-binding fragment thereof that binds BKB2R. The repertoire may then be displayed in a suitable host system such as the phage display system of WO92/01047 so that suitable antibodies or antigen-binding fragments thereof may be selected. A repertoire may consist of at least from about 10⁴ individual members and upwards by several orders of magnitude, for example, to about from 10⁶ to 10⁸ or 10¹⁰ or more members. Analogous shuffling or combinatorial techniques are also disclosed by Stemmer (Nature, 1994, 370:389-391), who describes the technique in relation to a β-lactamase gene but observes that the approach may be used for the generation of antibodies.

A further alternative is to generate novel VH or VL regions carrying one or more CDR-derived sequences of the herein described invention embodiments using random mutagenesis of one or more selected VH and/or VL genes to generate mutations within the entire variable domain. Such a technique is described by Gram et al. (1992 Proc. Natl. Acad. Sci. USA 89:3576-3580), who used error-prone PCR. Another method which may be used is to direct mutagenesis to CDR regions of VH or VL genes. Such techniques are disclosed by Barbas et al. (1994 Proc. Natl. Acad. Sci. USA 91:3809-3813) and Schier et al. (1996 J. Mol. Biol. 263:551-567).

In certain embodiments, a specific VH and/or VL of the antibodies described herein may be used to screen a library of the complementary variable domain to identify antibodies with desirable properties, such as increased affinity for BKB2R. Such methods are described, for example, in Portolano et al., J. Immunol. (1993) 150:880-887; and Clarkson et al., Nature (1991) 352:624-628.

Other methods may also be used to mix and match CDRs to identify antibodies having desired binding activity, such as binding to BKB2R. For example: Klimka et al., British Journal of Cancer (2000) 83: 252-260, describe a screening process using a mouse VL and a human VH library with CDR3 and FR4 retained from the mouse VH. After obtaining antibodies, the VH was screened against a human VL library to obtain antibodies that bound antigen. Beiboer et al., J. Mol. Biol. (2000) 296:833-849 describe a screening process using an entire mouse heavy chain and a human light chain library. After obtaining antibodies, one VL was combined with a human VH library with the CDR3 of the mouse retained. Antibodies capable of binding antigen were obtained. Rader et al., Proc. Nat. Acad. Sci. USA (1998) 95:8910-8915 describe a process similar to that of Beiboer et al above.

These just-described techniques are, in and of themselves, known as such in the art. Based on the present disclosure, the skilled person will, however, be able to use such techniques to obtain antibodies or antigen-binding fragments thereof according to several embodiments of the invention described herein, using routine methodology in the art.

Also disclosed herein is a method for obtaining an antibody antigen binding domain specific for BKB2R antigen, the method comprising providing, by way of addition, deletion, substitution or insertion of one or more amino acids in the amino acid sequence of a VH domain set forth herein, a VH domain which is an amino acid sequence variant of the VH domain. Optionally the VH domain thus provided may be combined with one or more VL domains. The VH domain, or VH/VL combination or combinations, may then be tested to identify a specific binding member or an antibody antigen binding domain specific for BKB2R, and optionally further having one or more preferred properties. Said VL domains may have an amino acid sequence which is substantially as set out herein. An analogous method may be employed in which one or more sequence variants of a VL domain disclosed herein are combined with one or more VH domains.

An epitope that “specifically binds” or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a particular BKB2R epitope is an antibody that binds one BKB2R epitope with greater affinity, avidity, more readily, and/or with greater duration than it binds to other BKB2R epitopes or to non-BKB2R epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}. See, generally, Davies et al. (1990) Annual Rev. Biochem. 59:439-473.

The term “immunologically active”, with reference to an epitope being or “remaining immunologically active”, refers to the ability of an antibody (e.g., anti-BKB2R antibody) to bind to the epitope under different conditions, for example, after the epitope has been subjected to reducing and denaturing conditions.

An antibody or antigen-binding fragment thereof according to certain preferred embodiments of the present application may be one that competes for binding to BKB2R with any antibody described herein which both (i) specifically binds to the antigen and (ii) comprises a VH and/or VL domain disclosed herein, or comprises a VH CDR3 disclosed herein, or a variant of any of these. Competition between binding members may be assayed easily in vitro, for example using ELISA and/or by tagging a specific reporter molecule to one binding member which can be detected in the presence of other untagged binding member(s), to enable identification of specific binding members which bind the same epitope or an overlapping epitope.

Thus, there is presently provided a specific antibody or antigen-binding fragment thereof, comprising an antibody antigen-binding site which competes with an antibody described herein that binds to BKB2R, such as the antibodies described in the Examples herein (e.g., clones 5F12G1 and humanized derivatives thereof, e.g., H1/L1, H2/L2, H37/L37, H38/L38; H39/L39).

The constant regions of immunoglobulins show less sequence diversity than the variable regions, and are responsible for binding a number of natural proteins to elicit important biochemical events. In humans there are five different classes of antibodies including IgA (which includes subclasses IgA1 and IgA2), IgD, IgE, IgG (which includes subclasses IgG1, IgG2, IgG3, and IgG4), and IgM. The distinguishing features between these antibody classes are their constant regions, although subtler differences may exist in the V region.

The Fc region of an antibody interacts with a number of Fc receptors and ligands, imparting an array of important functional capabilities referred to as effector functions. For IgG the Fc region comprises Ig domains CH2 and CH3 and the N-terminal hinge leading into CH2. An important family of Fc receptors for the IgG class are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65). These receptors typically have an extracellular domain that mediates binding to Fc, a membrane spanning region, and an intracellular domain that may mediate some signaling event within the cell. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack.

The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP). All FcγRs bind the same region on Fc, at the N-terminal end of the Cg2 (CH2) domain and the preceding hinge. This interaction is well characterized structurally (Sondermann et al., 2001, J Mol Biol 309:737-749), and several structures of the human Fc bound to the extracellular domain of human FcγRIIIb have been solved (pdb accession code 1E4K)(Sondermann et al., 2000, Nature 406:267-273.) (pdb accession codes 1IIS and 1IIX)(Radaev et al., 2001, J Biol Chem 276:16469-16477.)

The different IgG subclasses have different affinities for the FcγRs, with IgG1 and IgG3 typically binding substantially better to the receptors than IgG2 and IgG4 (Jefferis et al., 2002, Immunol Lett 82:57-65). All FcγRs bind the same region on IgG Fc, yet with different affinities: the high affinity binder FcγRI has a Kd for IgG1 of 10⁻⁸ M⁻¹, whereas the low affinity receptors FcγRII and FcγRIII generally bind at 10⁻⁶ and 10⁻⁵ respectively. The extracellular domains of FcγRIIIa and FcγRIIIb are 96% identical, however FcγRIIIb does not have a intracellular signaling domain. Furthermore, whereas FcγRI, FcγRIIa/c, and FcγRIIIa are positive regulators of immune complex-triggered activation, characterized by having an intracellular domain that has an immunoreceptor tyrosine-based activation motif (ITAM), FcγRIIb has an immunoreceptor tyrosine-based inhibition motif (ITIM) and is therefore inhibitory. Thus the former are referred to as activation receptors, and FcγRIIb is referred to as an inhibitory receptor. The receptors also differ in expression pattern and levels on different immune cells.

Yet another level of complexity is the existence of a number of FcγR polymorphisms in the human proteome. A particularly relevant polymorphism with clinical significance is V158/F158 FcγRIIIa. Human IgG1 binds with greater affinity to the V158 allotype than to the F158 allotype. This difference in affinity, and presumably its effect on ADCC and/or ADCP, has been shown to be a significant determinant of the efficacy of the anti-CD20 antibody rituximab (Rituxan®, a registered trademark of DEC Pharmaceuticals Corporation). Patients with the V158 allotype respond favorably to rituximab treatment; however, patients with the lower affinity F158 allotype respond poorly (Cartron et al., 2002 Blood 99:754-758). Approximately 10-20% of humans are V158/V158 homozygous, 45% are V158/F158 heterozygous, and 35-45% of humans are F158/F158 homozygous (Lehrnbecher et al., 1999 Blood 94:4220-4232; Cartron et al., 2002 Blood 99:754-758). Thus 80-90% of humans are poor responders, that is they have at least one allele of the F158 FcγRIIIa.

The Fc region is also involved in activation of the complement cascade. In the classical complement pathway, C1 binds with its C1q subunits to Fc fragments of IgG or IgM, which has formed a complex with antigen(s). In certain embodiments of the invention, modifications to the Fc region comprise modifications that alter (either enhance or decrease) the ability of a herein described BKB2R-specific antibody to activate the complement system (see e.g., U.S. Pat. No. 7,740,847). To assess complement activation, a complement-dependent cytotoxicity (CDC) assay may be performed (See, e.g., Gazzano-Santoro et al., J. Immunol. Meth. 202:163 (1996)). For example, various concentrations of the (Fc) variant polypeptide and human complement may be diluted with buffer. Mixtures of (Fc) variant antibodies, diluted human complement and cells expressing the antigen (BKB2R) may be added to a flat bottom tissue culture 96 well plate and allowed to incubate for 2 hours at 37° C. and 5% CO₂ to facilitate complement mediated cell lysis. Fifty microliters of alamar blue (Accumed International) may then be added to each well and incubated overnight at 37° C. The absorbance may be measured using a 96-well fluorimeter with excitation at 530 nm and emission at 590 nm. The results may be expressed in relative fluorescence units (RFU). The sample concentrations may be computed from a standard curve and the percent activity as compared to nonvariant antibody may be reported for the variant antibody of interest.

Thus in certain embodiments, the present invention provides anti-BKB2R antibodies having a modified Fc region with altered functional properties, such as enhanced ADCC, ADCP, CDC, or enhanced binding affinity for a specific FcγR. Illustrative modifications of the Fc region include those described in, e.g., Stavenhagen et al., 2007 Cancer Res. 67:8882. Other modified Fc regions contemplated herein are described, for example, in issued U.S. Pat. Nos. 7,317,091; 7,657,380; 7,662,925; 6,538,124; 6,528,624; 7,297,775; 7,364,731; Published U.S. Applications US2009092599; US20080131435; US20080138344; and published International Applications WO2006/105338; WO2004/063351; WO2006/088494; WO2007/024249.

The desired functional properties of anti-BKB2R antibodies may be assessed using a variety of methods known to the skilled person, including but not limited to calcium release by cells expressing BKB2R, affinity/binding assays (for example, surface plasmon resonance, competitive inhibition assays); cytotoxicity assays, cell viability assays (e.g., using dye exclusion such as Trypan Blue, propidium iodide, etc), cancer cell and/or tumor growth inhibition using in vitro or in vivo models (e.g., cell proliferation and/or colony formation assays; anchorage-dependent proliferation assays; standard human tumor xenograft models) (see, e.g., Culp P A, et al., Clin. Cancer Res. 16(2):497-508). Other assays may test the ability of antibodies described herein to block normal BKB2R-mediated responses, such as assays for intracellular glycogen synthesis and/or ELISA determination of GSK-3β phosphorylation at serine-9 as indicators of GSK-3β inhibition. Such assays may be performed based on the disclosure herein and knowledge in the art, for instance, using well-established protocols known to the skilled person (see e.g., Current Protocols in Molecular Biology (Greene Publ. Assoc. Inc. & John Wiley & Sons, Inc., NY, N.Y.); Current Protocols in Immunology (Edited by: John E. Coligan, Ada M. Kruisbeek, David H. Margulies, Ethan M. Shevach, Warren Strober 2001 John Wiley & Sons, NY, N.Y.); or commerially available kits.

In one embodiment, the anti-BKB2R antibodies described herein block binding of kinins (e.g., bradykinin and kallidin (Lys-bradykinin)) or any other ligand for BKB2R, to the BKB2R receptor. Binding assays and competitive inhibition assays may be used to determine blocking activity of the antibodies described herein, or variants or antigen-binding fragments thereof.

In certain embodiments, the anti-BKB2R antibodies described herein bind to BKB2R and stimulate, activate or otherwise induce downstream signaling events in the BKB2R signalling pathway. In particular embodiments, a level of BKB2R signaling stimulation provided by an anti-BKB2R antibody may be a statistically significant increase in the level of signaling via BKB2R of at least about 10%, at least about 25%, at least about 50%, at least about 60%, 65%, 70%, 75%, 80%, 85%, at least about 90%, or at least about 95%, 96%, 97%, 98%, 99% or 100% relative to the level of BKB2R signaling in the absence of the herein disclosed anti-BKB2R antibody. In certain embodiments, the statistically significant increase in the level of BKB2R signaling stimulation may be in excess of at least 100% greater than the level that is detectable in the absence of the herein disclosed anti-BKB2R antibody, which in some cases may be higher by 200%, 300% or more.

Thus, the present disclosure provides anti-BKB2R antibodies that modulate components of the GSK-3β signalling pathway. By modulate it is meant to alter activity, protein level, gene expression level, or phosphorylation state of a component of the GSK-3β signalling pathway in a statistically significant manner (e.g., to inhibit in a statistically significant manner, or to increase in a statistically signficant manner, as measured using appropriate controls). A component of the BKB2R G protein coupled receptor induces downstream signalling events via the PI3K/Akt signalling pathway, which includes, but is not limited to, phosphorylation and deactivation of GSK-3β on serine-9.

In certain embodiments, modulation of components of the BKB2R signalling pathway may comprise modulation of the phosphorylation state of one or more components of the pathway. In certain embodiments, binding of the anti-BKB2R antibodies of the present invention to the BKB2R receptor may cause, in a statistically significant manner, increased phosphorylation of GSK-36 on serine-9 and its deactivation.

In vivo and in vitro assays for determining whether an antibody alters (e.g., increases or decreases in a statistically significant manner) BKB2R signaling are known in the art. For example, cell-based assays such as induced calcium mobilization assays, or assays utilizing immunochemical detection of a BKB2R-related pathway component, such as GSK-3β, in cell lysates following induction with the herein described anti-BKB2R antibodies or other relevant stimuli, may be used to measure BKB2R signaling levels in vitro (e.g., Assay Designs® GSK-3β enzyme immunometric assay, Assay Designs, Inc., Ann Arbor, Mich.). Examples of such assays are also described herein in Examples 1 and 9. The level of BKB2R signaling in the presence of BKB2R ligands such as BK or kallidin when the BKB2R-binding antibody is present may also be compared to the level of signaling without the BKB2R-binding antibody being present. Non-limiting, specific examples of the use of cell-based assays to assess an effect of an anti-BKB2R monoclonal antibody on BKB2R signaling are provided in the Examples herein. In addition, the effect of a BKB2R-binding antibody on signaling may be measured in vitro or in vivo by measuring the effect of the antibody on the level of expression of genes that are regulated by components of BKB2R-related pathways, such as one or more of the recognized pathways in which GSK-3β participates. Other assays and commercially available systems for determining modulation of components of the BKB2R signalling pathway are known to the skilled person.

The present invention provides, in certain embodiments, an isolated nucleic acid encoding an antibody or antigen-binding fragment thereof as described herein, for instance, a nucleic acid which codes for a CDR or VH or VL domain. Nucleic acids include DNA and RNA. These and related embodiments may include polynucleotides encoding antibodies that bind BKB2R as described herein. The term “isolated polynucleotide” as used herein shall mean a polynucleotide of genomic, cDNA, or synthetic origin or some combination thereof, which by virtue of its origin the isolated polynucleotide (1) is not associated with all or a portion of a polynucleotide in which the isolated polynucleotide is found in nature, (2) is linked to a polynucleotide to which it is not linked in nature, or (3) does not occur in nature as part of a larger sequence.

The term “operably linked” means that the components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein refers to polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

The term “polynucleotide” as referred to herein means single-stranded or double-stranded nucleic acid polymers. In certain embodiments, the nucleotides comprising the polynucleotide can be ribonucleotides or deoxyribonucleotides or a modified form of either type of nucleotide. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, Anti-Cancer Drug Design, 6:539; Zon et al., 1991, OLIGONUCLEOTIDES AND ANALOGUES: A PRACTICAL APPROACH, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby incorporated by reference for any purpose. An oligonucleotide can include a detectable label to enable detection of the oligonucleotide or hybridization thereof.

The term “vector” is used to refer to any molecule (e.g., nucleic acid, plasmid, or virus) used to transfer coding information to a host cell. The term “expression vector” refers to a vector that is suitable for transformation of a host cell and contains nucleic acid sequences that direct and/or control expression of inserted heterologous nucleic acid sequences. Expression includes, but is not limited to, processes such as transcription, translation, and RNA splicing, if introns are present.

As will be understood by those skilled in the art, polynucleotides may include genomic sequences, extra-genomic and plasmid-encoded sequences and smaller engineered gene segments that express, or may be adapted to express, proteins, polypeptides, peptides and the like. Such segments may be naturally isolated, or modified synthetically by the skilled person.

As will also be recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence that encodes a variant or derivative of such a sequence.

Therefore, according to these and related embodiments, polynucleotides are provided that comprise some or all of a polynucleotide sequence set forth in any one or more of SEQ ID NOs:49-60, 76, 78, 80 and 82, complements of a polynucleotide sequence set forth in any one or more of SEQ ID NOs: 49-60, 76, 78, 80 and 82, and degenerate variants of a polynucleotide sequence set forth in any one or more of SEQ ID NOs: 49-60, 76, 78, 80 and 82. In certain preferred embodiments, the polynucleotide sequences set forth herein encode antibodies, or antigen-binding fragments thereof, which bind the BKB2R, as described elsewhere herein. In certain preferred embodiments, the polynucleotide sequences set forth herein encode polypeptides having the amino acid sequences set forth in SEQ ID NOS:1-48, 75, 77, 79, 81, and 83-92.

In other related embodiments, polynucleotide variants may have substantial identity to the sequences disclosed herein in SEQ ID NOs: 49-60, 76, 78, 80 and 82, for example those comprising at least 70% sequence identity, preferably at least 75%, 80%, 85%, 90%, 95%, 96%, 97%, 98%, or 99% or higher, sequence identity compared to a reference polynucleotide sequence such as the sequences disclosed herein, using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the binding affinity of the antibody encoded by the variant polynucleotide is not substantially diminished relative to an antibody encoded by a polynucleotide sequence specifically set forth herein.

In certain other related embodiments, polynucleotide fragments may comprise or consist essentially of various lengths of contiguous stretches of sequence identical to or complementary to one or more of the sequences disclosed herein. For example, polynucleotides are provided that comprise or consist essentially of at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of the disclosed sequence or at both ends of the disclosed sequence.

In another embodiment, polynucleotides are provided that are capable of hybridizing under moderate to high stringency conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide as provided herein with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2×, 0.5× and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60-65° C. or 65-70° C.

In certain embodiments, the polynucleotides described above, e.g., polynucleotide variants, fragments and hybridizing sequences, encode antibodies that bind BKB2R, or antigen-binding fragments thereof. In other embodiments, such polynucleotides encode antibodies or antigen-binding fragments, or CDRs thereof, that bind to BKB2R at least about 50%, preferably at least about 70%, and more preferably at least about 90% as well as an antibody sequence specifically set forth herein. In further embodiments, such polynucleotides encode antibodies or antigen-binding fragments, or CDRs thereof, that bind to BKB2R with greater affinity than the antibodies set forth herein, for example, that bind quantitatively at least about 105%, 106%, 107%, 108%, 109%, or 110% as well as an antibody sequence specifically set forth herein.

Determination of the three-dimensional structures of representative polypeptides (e.g., variant BKB2R-specific antibodies as provided herein, for instance, an antibody protein having an antigen-binding fragment as provided herein) may be made through routine methodologies such that substitution, addition, deletion or insertion of one or more amino acids with selected natural or non-natural amino acids can be virtually modeled for purposes of determining whether a so derived structural variant retains the space-filling properties of presently disclosed species. See, for instance, Donate et al., 1994 Prot. Sci. 3:2378; Bradley et al., Science 309: 1868-1871 (2005); Schueler-Furman et al., Science 310:638 (2005); Dietz et al., Proc. Nat. Acad. Sci. USA 103:1244 (2006); Dodson et al., Nature 450:176 (2007); Qian et al., Nature 450:259 (2007); Raman et al. Science 327:1014-1018 (2010). Some additional non-limiting examples of computer algorithms that may be used for these and related embodiments, such as for rational design of BKB2R-specific antibodies antigen-binding domains thereof as provided herein, include NAMD, a parallel molecular dynamics code designed for high-performance simulation of large biomolecular systems, and VMD which is a molecular visualization program for displaying, animating, and analyzing large biomolecular systems using 3-D graphics and built-in scripting (see Phillips, et al., Journal of Computational Chemistry, 26:1781-1802, 2005; Humphrey, et al., “VMD—Visual Molecular Dynamics”, J. Molec. Graphics, 1996, vol. 14, pp. 33-38; see also the website for the Theoretical and Computational Biophysics Group, University of Illinois at Urbana-Champagne, at ks.uiuc.edu/Research/vmd/). Many other computer programs are known in the art and available to the skilled person and which allow for determining atomic dimensions from space-filling models (van der Waals radii) of energy-minimized conformations; GRID, which seeks to determine regions of high affinity for different chemical groups, thereby enhancing binding, Monte Carlo searches, which calculate mathematical alignment, and CHARMM (Brooks et al. (1983) J. Comput. Chem. 4:187-217) and AMBER (Weiner et al (1981) J. Comput. Chem. 106: 765), which assess force field calculations, and analysis (see also, Eisenfield et al. (1991) Am. J. Physiol. 261:C376-386; Lybrand (1991) J. Pharm. Belg. 46:49-54; Froimowitz (1990) Biotechniques 8:640-644; Burbam et al. (1990) Proteins 7:99-111; Pedersen (1985) Environ. Health Perspect. 61:185-190; and Kini et al. (1991) J. Biomol. Struct. Dyn. 9:475-488). A variety of appropriate computational computer programs are also commercially available, such as from Schrödinger (Munich, Germany).

The polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., Unified Approach to Alignment and Phylogenes, pp. 626-645 (1990); Methods in Enzymology vol. 183, Academic Press, Inc., San Diego, Calif.; Higgins, D. G. and Sharp, P. M., CABIOS 5:151-153 (1989); Myers, E. W. and Muller W., CABIOS 4:11-17 (1988); Robinson, E. D., Comb. Theor 11:105 (1971); Santou, N. Nes, M., Mol. Biol. Evol. 4:406-425 (1987); Sneath, P. H. A. and Sokal, R. R., Numerical Taxonomy—the Principles and Practice of Numerical Taxonomy, Freeman Press, San Francisco, Calif. (1973); Wilbur, W. J. and Lipman, D. J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

Preferred examples of algorithms that are suitable for determining percent sequence identity and sequence similarity are the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the parameters described herein, to determine percent sequence identity among two or more the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff, Proc. Natl. Acad. Sci. USA 89:10915 (1989)) alignments, (B) of 50, expectation (E) of 10, M=5, N=−4 and a comparison of both strands.

In certain embodiments, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode an antibody as described herein. Some of these polynucleotides bear minimal sequence identity to the nucleotide sequence of the native or original polynucleotide sequence, such as those described herein that encode antibodies that bind to BKB2R. Nonetheless, polynucleotides that vary due to differences in codon usage are expressly contemplated by the present disclosure. In certain embodiments, sequences that have been codon-optimized for mammalian expression are specifically contemplated.

Therefore, in another embodiment of the invention, a mutagenesis approach, such as site-specific mutagenesis, may be employed for the preparation of variants and/or derivatives of the antibodies described herein. By this approach, specific modifications in a polypeptide sequence can be made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments, the inventors contemplate the mutagenesis of the disclosed polynucleotide sequences to alter one or more properties of the encoded polypeptide, such as the binding affinity of the antibody or the antigen-binding fragment thereof, or the function of a particular Fc region, or the affinity of the Fc region for a particular FcγR. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maniatis et al., 1982, infra, and other sources cited below for molecular biology and molecular genetic and related methodologies, each incorporated herein by reference for that purpose.

The term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. The term “oligonucleotide directed mutagenesis procedure” is intended to refer to a process that involves the template-dependent extension of a primer molecule. The term “template dependent process” refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide variants, recursive sequence recombination, as described in U.S. Pat. No. 5,837,458, may be employed. In this approach, iterative cycles of recombination and screening or selection are performed to “evolve” individual polynucleotide variants having, for example, increased binding affinity. Certain embodiments also provide constructs in the form of plasmids, vectors, transcription or expression cassettes which comprise at least one polynucleotide as described herein.

According to certain related embodiments there is provided a recombinant host cell which comprises one or more constructs as described herein; a nucleic acid encoding any antibody, CDR, VH or VL domain, or antigen-binding fragment thereof; and a method of production of the encoded product, which method comprises expression from encoding nucleic acid therefor. Expression may conveniently be achieved by culturing under appropriate conditions recombinant host cells containing the nucleic acid. Following production by expression, an antibody or antigen-binding fragment thereof, may be isolated and/or purified using any suitable technique, and then used as desired.

Antibodies or antigen-binding fragments thereof as provided herein, and encoding nucleic acid molecules and vectors, may be isolated and/or purified, e.g. from their natural environment, in substantially pure or homogeneous form, or, in the case of nucleic acid, free or substantially free of nucleic acid or genes of origin other than the sequence encoding a polypeptide with the desired function. Nucleic acid may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli.

The expression of antibodies and antigen-binding fragments in prokaryotic cells such as E. coli is well established in the art. For a review, see for example Pluckthun, Bio/Technology 9: 545-551 (1991). Expression in eukaryotic cells in culture is also available to those skilled in the art as an option for production of antibodies or antigen-binding fragments thereof, see recent reviews, for example Ref, (1993) Curr. Opinion Biotech. 4: 573-576; Trill et al. (1995) Curr. Opinion Biotech 6: 553-560.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press; see also additional references cited below pertaining to molecular biology methods. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992, or subsequent updates thereto.

The term “host cell” is used to refer to a cell into which has been introduced, or which is capable of having introduced into it, a nucleic acid sequence encoding one or more of the herein described antibodies, and which further expresses or is capable of expressing a selected gene of interest, such as a gene encoding any herein described antibody. The term includes the progeny of the parent cell, whether or not the progeny are identical in morphology or in genetic make-up to the original parent, so long as the selected gene is present. Accordingly there is also contemplated a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene. In one embodiment, the nucleic acid is integrated into the genome (e.g. chromosome) of the host cell. Integration may be promoted by inclusion of sequences which promote recombination with the genome, in accordance-with standard techniques.

The present invention also provides, in certain embodiments, a method which comprises using a construct as stated above in an expression system in order to express a particular polypeptide such as a BKB2R-specific antibody as described herein. The term “transduction” is used to refer to the transfer of genes from one bacterium to another, usually by a phage. “Transduction” also refers to the acquisition and transfer of eukaryotic cellular sequences by retroviruses. The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, MOLECULAR CLONING, A LABORATORY MANUAL, Cold Spring Harbor Laboratories; Davis et al., 1986, BASIC METHODS 1N MOLECULAR BIOLOGY, Elsevier; and Chu et al., 1981, Gene 13:197. Such techniques can be used to introduce one or more exogenous DNA moieties into suitable host cells.

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell, or may be maintained transiently as an episomal element without being replicated, or may replicate independently as a plasmid. A cell is considered to have been stably transformed when the DNA is replicated with the division of the cell. The term “naturally occurring” or “native” when used in connection with biological materials such as nucleic acid molecules, polypeptides, host cells, and the like, refers to materials which are found in nature and are not manipulated by a human. Similarly, “non-naturally occurring” or “non-native” as used herein refers to a material that is not found in nature or that has been structurally modified or synthesized by a human.

The terms “polypeptide” “protein” and “peptide” and “glycoprotein” are used interchangeably and mean a polymer of amino acids not limited to any particular length. The term does not exclude modifications such as myristylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. The terms “polypeptide” and “protein” specifically encompass the antibodies that bind to BKB2R of the present disclosure, or sequences that have deletions from, additions to, and/or substitutions of one or more amino acid of an anti-BKB2R antibody. Thus, a “polypeptide” or a “protein” can comprise one (termed “a monomer”) or a plurality (termed “a multimer”) of amino acid chains.

The term “isolated” with respect to a protein referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or noncovalent interaction) with portions of a protein with which the “isolated protein” is associated in nature, (6) is operably associated (by covalent or noncovalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise).

The term “polypeptide fragment” refers to a polypeptide, which can be monomeric or multimeric, that has an amino-terminal deletion, a carboxyl-terminal deletion, and/or an internal deletion or substitution of a naturally-occurring or recombinantly-produced polypeptide. In certain embodiments, a polypeptide fragment can comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long. Particularly useful polypeptide fragments include functional domains, including antigen-binding domains or fragments of antibodies. In the case of an anti-BKB2R antibody, useful fragments include, but are not limited to: a CDR region, especially a CDR3 region of the heavy or light chain; a variable domain of a heavy or light chain; a portion of an antibody chain or just its variable region including two CDRs; and the like.

BKB2R-binding antibodies or antigen-binding fragments thereof as described herein which are modulators, agonists or antagonists of BKB2R function are expressly included within the contemplated embodiments. These agonists, antagonists and modulator antibodies or antigen-binding fragments thereof interact with one or more of the antigenic determinant sites of BKB2R, or epitope fragments or variants of BKB2R.

As would be recognized by the skilled person, there are many known methods for making antibodies that bind to a particular antigen, such as BKB2R, including standard technologies, see, e.g., Harlow and Lane, Antibodies: A Laboratory Manual, Cold Spring Harbor Laboratory, 1988. In general, antibodies, such as antibodies that specifically block binding of the BKB2R-binding antibodies expressly disclosed herein to their cognate antigens, can be produced by cell culture techniques, including the generation of monoclonal antibodies as described herein, or via transfection of antibody genes into suitable bacterial or mammalian cell hosts, in order to allow for the production of recombinant antibodies. In certain embodiments, an immunogen comprising a polypeptide antigen (e.g., human BKB2R protein comprising the amino acid sequence as set forth in SEQ ID NO:71, or a fragment thereof such as the polypeptide comprising the amino acid sequence set forth in SEQ ID NO:73) is initially injected into any of a wide variety of mammals (e.g., mice, rats, rabbits, sheep or goats). In this step, the polypeptide may serve as the immunogen without modification. Alternatively, particularly for relatively short polypeptides, a superior immune response may in some cases be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

In certain embodiments, monoclonal antibodies specific for an antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides may be used in the purification process in, for example, an affinity chromatography step.

### Methods of Use and Pharmaceutical Compositions

Provided herein are methods of treatment using the antibodies that bind BKB2R. In one embodiment, an antibody of the present invention is administered to a patient having a disease, disorder or condition involving a biological signaling pathway the activity of which may be altered (e.g., increased or decreased in a statistically significant manner) by agonizing the BKB2R, which is meant in the context of the present disclosure to include diseases and disorders characterized by aberrant BKB2R and/or GSK-3β activity, due for example to alterations (e.g., statistically significant increases or decreases) in the amount or activity of a protein that is present, or the presence of a mutant protein, or both. An overabundance may be due to any cause, including but not limited to overexpression at the molecular level, prolonged or accumulated appearance at the site of action, or increased (e.g., in a statistically significant manner) activity of GSK-3β relative to that which is normally detectable. Such an overabundance of GSK-3β activity can be measured relative to normal expression, appearance, or activity of GSK-3β, and said measurement may play an important role in the development and/or clinical testing of the antibodies described herein.

In particular, the present antibodies described herein are useful for the treatment of diabetes and specifically certain complications of diabetes, by binding to BKB2R and subsequent signalling events. Thus, in certain embodiments, the antibodies described herein are useful for the treatment of diseases associated with diabetes including type 2 diabetes, such as, impaired glucose tolerance, insulin resistance, or other related disorders or conditions, including associated symptoms, hypercholesterolemia, hypertriglyceridemia, cardiovascular disease, hypertension, nephropathy, retinopathy and neuropathy.

In type II diabetes, resistance to insulin results in the lack of glucose uptake by tissues such as skeletal muscles. The insulin-resistance results in higher blood glucose levels, and the pancreas produces more insulin to compensate for the higher blood glucose levels. Exercise studies have discovered the connection between insulin-resistance, skeletal muscle glucose uptake and the BKB2R. During exercise, within skeletal muscles there is a localized increase in kinin release. This increase results in the increased muscle cell surface expression of the glucose transporter GLUT-4 and improved glucose uptake into muscle cells (Kishi et al, 1998 Diabetes 47:4, 550-8). Insulin resistance of muscle cells has been shown to be improved by the addition of kinins that act on the BKB2R for type II diabetes (Henriksen et al., 1998 Am J Physiol 275(1 Pt 2):R40-5.

In animal models of Type 2 diabetes insulin resistance, overactive glycogen synthase kinase-3 beta (GSK-3β) was found to be responsible for insulin resistance. Down regulation of GSK-3β resulted in reduced insulin resistance and improve glucose utilization by the body (Tanabe et al, 2007 PLos Biol 6:307-318). Although primarily an autoimmune based disease, Type I diabetes is now being recognized as having an insulin resistance component as well (Xu, et al., 2007 Diabetes Care 30:2314-20). Insulin resistance may be diagnosed via a hyperinsulinemic-euglycemic clamp. The BKB2R antibodies of certain of the instant invention embodiments may be administered to diabetic patients exhibiting insulin resistance.

The complications of diabetes, type 1 and type 2, may include the results of long term hyperglycemia and insulin resistance leading to severe damage to the kidneys (nephropathy), eyes (retinopathy), and/or nerves (neuropathy), and may additionally or alternatively include hypercholesterolemia and/or hypertension that lead to cardiovascular disease (e.g., myocardial infarction, cardiomyopathy and stroke). Activation of the BKB2R has been shown to contribute significantly to the protection of the kidneys against diabetic nephropathy (Allard et al. 2008 Am J Physiol Renal Physiology 294:F1249-56; Yuan et al, 2007 Endocrinology 148; 2016-2026) and certain BKB2K polymorphisms increase the risk of diabetic nephropathy (Maltais et al, 2002 Can J Physiol Pharmacol 80:323-7). BKB2R expression appears to play an important role in diabetic retinopathy and activation of BKB2R should improve diabetic retinopathy (Kato et al. 2009 Eur J Pharamcol 606:187-90) and neuropathy as well (Kakoki et al, 2010 Proc Natl Acd Sci USA 107:10190-5). The presently provided anti-BKB2R antibodies thus may, according to certain contemplated embodiments, be administered to diabetic patients to reverse or prevent further development of nephropathy, neuropathy or retinopathy.

Diabetes is also associated with cardiovascular disease. Tissue kallikrein, via activation of the bradykinin B2 receptor (BKB2R), plays an important role in cardioprotection. Bradykinin B2 receptor knock-out mice were shown to develop dilated cardiomyopathy in association with perivascular and reparative fibrosis (Emanueli et al., 1999 Circulation, 100; 2359-2365). Systemic delivery of adenovirus carrying the tissue kallikrein gene led to blood pressure reduction and attenuation of cardiac hypertrophy and fibrosis in hypertensive rats (Chao et al 1999 Stroke; 30; 1925-1932). Moreover, kallikrein gene transfer attenuated cardiac hypertrophy and fibrosis in normotensive rats after myocardial infarction and in genetically hypertensive rats without apparently affecting blood pressure. Furthermore, BKB2R activation improved cardiac function and reduced infarct size after myocardial infarction and the incidence of ventricular fibrillation; icatibant abolished these beneficial effects (Yin et al., 2005 J. Biol. Chem. 280, 8022-8030). The use of a BKB2R peptide agonist after myocardial infarction has also been noted to confer a beneficial effect on cardiac function (Marketou et al, 2010 Am J Hypertens 23:562-568). Kinin protects against ischemia/reperfusion-induced cardiomyocyte apoptosis in vivo and in cultured cells via stimulation of kinin B2 receptor-Akt-GSK-3b and Akt-Bad-14-3-3 signaling pathways. In addition, nitric oxide (NO) plays an important role in BKB2R-mediated protection against myocardial ischemia/reperfusion-induced inflammation and ventricular remodeling by suppression of oxidative stress, TGF-b1/Smad2 and JNK/p38MAPK signaling pathways and NF-kB activation. These findings indicate that kallikrein protects against cardiac injury and improves cardiac function with or without affecting blood pressure. Taken together, the results from in vivo and in vitro studies indicate that tissue kallikrein, through BKB2R activation, protects against cardiac injury by inhibiting apoptosis, inflammation, hypertrophy and fibrosis through increasing NO formation and suppressing oxidative stress-mediated signaling cascades. The anti-BKB2R antibodies described herein therefore may, according to certain expressly contemplated embodiments, be administered to diabetic patients to reverse or prevent further development of cardiovascular disease.

Another embodiment provides a method for inhibiting GSK-313 pathway signalling in a cell expressing BKB2R by contacting the cell with an amount of a herein disclosed BKB2R-specific antibody sufficient to decrease cholesterol levels. By way of a brief background, hypercholesterolemia occurs when the presence of cholesterol in the blood is very high. Long-term hypercholesterolemia results in cardiovascular disease with hardening of the arteries (atherosclerosis) and a higher risk of myocardial infraction and stroke. Total cholesterol concentrations in the circulation of less than 200 mg/dL are desirable, however, between 200-239 mg/dL is typically regarded as a borderline high level and above 240 mg/dL is considered high. In order to reduce the risks of cardiovascular disease, total cholesterol may desirably be lowered to less than 200 mg/dL, in which LDL cholesterol should be ideally below 100 mg/dL, or below 70 mg/dL for those at very high risk, and HDL cholesterol below 40 mg/dL. Although diet and exercise may contribute to lowering total cholesterol levels, such a regimen alone is not always successful and thus additional drug therapy may be indicated. Subjects having diabetes are considered to be at high risk, and thus are typically advised to carefully control cholesterol levels. Kallikrein via the BKB2R activation also protected against cardiomyopathy by improving cardiac function, serum glucose and lipid profiles, including cholesterol, in streptozotocin-induced diabetic rats (Montanari et al., 2005 Diabetes 54; 1573-1580). In a type 2 diabetes, high fat diet animal model, introduction of the tissue kallikrein gene expression via a recombinant retrovirus led to significant reduction in total cholesterol levels compared to untreated animals (Yuan, G, et al, 2007 Endocrinology 148; 2016-2026). Accordingly, therapeutic intervention as disclosed herein, by administration of the present agonistic anti-BKB2R antibody, is contemplated according to certain embodiments, to beneficially decrease circulating cholesterol levels.

With regard to treatment of hypertension with the herein described antibody according to certain other embodiments, it is known that kinins (Lys-bradykinin and bradykinin) bind to the constitutively expressed cell surface receptor BKB2R (bradykinin type 2 receptor), leading to smooth muscle relaxation in blood vessels which results in a drop in blood pressure. Angiotensin converting enzyme (ACE) counters the hypotensive properties of these kinins by further metabolizing them so that they can no longer bind to the BKB2R. The importance of the BKB2R in blood pressure regulation is further highlighted by an increase in blood pressure when receptor expression is knocked out (Madeddu et al, 1996 Hypertension 28:980-987). In another study, the over expression of tissue kallikrein acting through the BKB2R in a hypertension animal model led to sustained reductions in blood pressure (Wang et al 1995 J Clin Invest. 95: 1710-1760). The BKB2R antibodies described herein thus may, in these and related embodiments, be administered to patients to treat hypertension.

In particular, the present antibodies are useful for the treatment of a variety of cancers associated with the expression and/or activity of BKB2R and/or GSK-3β. For example, one embodiment of the invention provides a method for the treatment of a cancer including, but not limited to, mixed lineage leukemia, esophageal cancer, ovarian cancer, prostate cancer, kidney cancer, colon cancer, liver cancer, stomach cancer, and pancreatic cancer, by administering to a cancer patient a therapeutically effective amount of a herein disclosed BKB2R-specific antibody. An amount that, following administration, inhibits, prevents or delays the progression and/or metastasis of a cancer in a statistically significant manner (i.e., relative to an appropriate control as will be known to those skilled in the art) is considered effective.

Another embodiment provides a method for inhibiting the GSK-3β pathway signalling in a cell expressing BKB2R by contacting the cell with an amount of a herein disclosed BKB2R-specific antibody sufficient to inhibit signalling and inhibit the growth of cancer cells. Certain cancers have been determined to be sensitive to glycogen synthase kinase-3 beta (GSK-3β) inhibition. Specifically, pancreatic carcinoma, hepatocellular carcinoma, gastric cancer and colorectal cancer were shown to have increased GSK-3β expression compared to non-neoplastic tissues. Inhibition of GSK-3β resulted in attenuated survival and proliferation of the cancer cells, and increased apoptosis in cell culture and in xenografts in mice (Mai et al, Clin Cancer Res 2009; 15(22) 6810-6819). The anti-BKB2R antibodies described herein were also effective in inhibiting the growth of cell lines derived from hepatocellular carcinoma, gastric cancer and colorectal cancer. In esophageal cancer, GSK-313 inhibition similarly resulted in cell cycle arrest of the cell line in culture (Wang et al, Worl J Gastroenterol, 2008; 14(25): 3982-3989).

In prostate cancer, inhibition of GSK-3β repressed expression of the androgen receptor and inhibited growth of the prostate cancer cell lines (Mazor et al, Oncogene 2004; 23; 7882-7892). In ovarian cancer, GSK-3β activity was involved in the proliferation of human ovarian cancer cells both in culture and in an animal model. Inhibition of GSK-3β prevented the formation in nude mice of tumors generated from human ovarian cancer cell line (Cao et al, 2006 Cell Research; 16; 671-677). In MLL (myeloid/lymphoid or mixed lineage leukemia) GSK-3β has been demonstrated as an oncogenic requirement for maintenance of human leukemia with mutations in the MLL proto-oncogene. Inhibition of GSK-3β resulted in cell cycle arrest of several MLL cell lines in culture. In a preclinical murine model of human MLL leukemia, GSK-3β inhibition resulted in significant prolongation of survival of the mice (Wang et al, 2008 Nature; 455; 1205-1210). The anti-BKB2R antibodies described herein were effective in inhibiting the growth of cell lines derived from prostate cancer and MML leukemia.

Another embodiment provides a method for inhibiting GSK-3β pathway signalling in a cell expressing BKB2R by contacting the cell with an amount of a herein disclosed anti-BKB2R-specific antibody sufficient to counteract exposure to radiation. Exposure to radiation from a variety of sources (nuclear accident, nuclear weapon detonation, cancer radiation therapy) can lead to very severe and life-threatening physical and neurological deficits. Inhibition of GSK-3β may be a way to counteract the exposure to radiation at the cellular level and has been noted to help overcome neurological deficits from cancer radiation therapy (Yazlovtskaya et al, 2006 Cancer Res 66:11179-86).

Another embodiment provides a method for inhibiting GSK-3β pathway signalling in a cell expressing BKB2R by contacting the cell with an amount of a herein disclosed BKB2R-specific antibody sufficient to counteract exposure to influenza virus infection. Influenza virus infection of the respiratory tract is a majory cause of illness and death worldwide each year. Currently, anti-viral therapeutics, such as Oseltamivir, when used against influenza, are becoming ineffective due to the rapid mutation of rate of the virus. The influenza virus relies on host cell machinery for viral entry and replication. One of the identified host cell proteins required by influenza is GSK-3β (Konig, R, et al, (2010) Nature 463:813-817), and knocking out GSK-3β expression with siRNAs, led to a large reduction in viral replication. Certain herein disclosed embodiments, by inhibiting GSK-3β through the agonist signaling activity of the presently provided anti-BKB2R antibodies, therefore contemplate a therapeutic approach to the treatment of influenze virus infections that, according to non-limiting theory, are not likely to result in viral resistance.

Another embodiment provides a method for inhibiting GSK-3β pathway signalling in a cell expressing BKB2R by contacting the cell with an amount of a herein disclosed BKB2R-specific antibody sufficient to inhibit signalling via the GSK-3β pathway for the treatment of stroke patients. An ischemic stroke occurs when a blood vessel to the brain is blocked by a blood clot, resulting in no blood flow to the brain. The loss of blood flow to the brain results in damage to brain tissue in a particular area leading to debilitating injury. The BKB2R is known for its protective role in ischemic stroke. Infarct volume and neurological deficit scores were found to be more pronounced in BKB2R-deficient mice compared to normal mice using the MCAO ischemic stroke model (Chao et al, 2006 Front Biosci 11:1323-7). Survival rates were also found to be lower in the BKB2R deficient mice. Hence, certain presently disclosed embodiments relate to methods for treating stroke by administering the herein described anti-BKB2R antibodies.

Administration of the BKB2R-specific antibodies described herein, in pure form or in an appropriate pharmaceutical composition, can be carried out via any of the accepted modes of administration of agents for serving similar utilities. The pharmaceutical compositions can be prepared by combining an antibody or antibody-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients and/or suitable excipients such as salts, buffers and stabilizers may, but need not, be present within the composition. Administration may be achieved by a variety of different routes, including oral, parenteral, nasal, intravenous, intradermal, subcutaneous or topical. Preferred modes of administration depend upon the nature of the condition to be treated or prevented. An amount that, following administration, reduces, inhibits, prevents or delays the progression and/or metastasis of a cancer is considered effective.

In certain embodiments, the amount administered is sufficient to result in reduced blood pressure, and/or decreased blood glucose concentrations, and/or decreased serum cholesterol concentrations, and/or reduced viral load, and/or tumor regression, and/or reduced risk of cardiovascular disease, retinopathy, neuropathy or nephropathy, and/or reduced morbidity or mortality following stroke or radiation exposure, as indicated by a statistically significant decrease in one or more of the particular parameters for which therapeutic intervention is indicated. The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described BKB2R-specific antibody in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of an antibody of the present disclosure, for treatment of a disease or condition of interest in accordance with teachings herein.

A pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should contain an amount of an BKB2R-specific antibody as herein disclosed such that a suitable dosage will be obtained. Typically, this amount is at least 0.01% of the antibody in the composition. When intended for oral administration, this amount may be varied to be between 0.1 and about 70% of the weight of the composition. Certain oral pharmaceutical compositions contain between about 4% and about 75% of the antibody. In certain embodiments, pharmaceutical compositions and preparations according to the present invention are prepared so that a parenteral dosage unit contains between 0.01 to 10% by weight of the antibody prior to dilution.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The pharmaceutical composition in solid or liquid form may include an agent that binds to the antibody of the invention and thereby assists in the delivery of the compound. Suitable agents that may act in this capacity include other monoclonal or polyclonal antibodies, one or more proteins or a liposome. The pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The pharmaceutical compositions may be prepared by methodology well known in the pharmaceutical art. For example, a pharmaceutical composition intended to be administered by injection can be prepared by combining a composition that comprises a herein-described BKB2R-specific antibody and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the antibody composition so as to facilitate dissolution or homogeneous suspension of the antibody in the aqueous delivery system.

The compositions may be administered in a therapeutically effective amount, which will vary depending upon a variety of factors including the activity of the specific compound (e.g., BKB2R-specific antibody) employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 kg mammal) from about 0.001 mg/kg (i.e., 0.07 mg) to about 100 mg/kg (i.e., 7.0 g); preferaby a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g).

The compositions comprising herein described BKB2R-specific antibodies may be administered to an individual afflicted with a disease as described herein, such as a cancer. For in vivo use for the treatment of human disease, the antibodies described herein are generally incorporated into a pharmaceutical composition prior to administration. A pharmaceutical composition comprises one or more of the antibodies described herein in combination with a physiologically acceptable carrier or excipient as described elsewhere herein. To prepare a pharmaceutical composition, an effective amount of one or more of the compounds is mixed with any pharmaceutical carrier(s) or excipient known to those skilled in the art to be suitable for the particular mode of administration. A pharmaceutical carrier may be liquid, semi-liquid or solid. Solutions or suspensions used for parenteral, intradermal, subcutaneous or topical application may include, for example, a sterile diluent (such as water), saline solution, fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously, suitable carriers include physiological saline or phosphate buffered saline (PBS), and solutions containing thickening and solubilizing agents, such as glucose, polyethylene glycol, polypropylene glycol and mixtures thereof.

The compositions comprising BKB2R-specific antibodies as described herein may be prepared with carriers that protect the antibody against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

As used herein the singular forms “a”, “an” and “the” include plural aspects unless the context clearly dictates otherwise. Thus, for example, reference to “a cell” includes a single cell, as well as two or more cells; reference to “an agent” includes one agent, as well as two or more agents; and so forth.

Each embodiment described in this specification is to be applied mutatis mutandis to every other embodiment unless expressly stated otherwise.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references in microbiology, molecular biology, biochemistry, molecular genetics, cell biology, virology and immunology techniques that are cited and discussed throughout the present specification. See, e.g., Sambrook, et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, N.Y.); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3^{rd} Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and CC Blackwell, eds., 1986); Riott, Essential Immunology, 6th Edition, (Blackwell Scientific Publications, Oxford, 1988); Embryonic Stem Cells: Methods and Protocols (Methods in Molecular Biology) (Kurstad Turksen, Ed., 2002); Embryonic Stem Cell Protocols: Volume I: Isolation and Characterization (Methods in Molecular Biology) (Kurstad Turksen, Ed., 2006); Embryonic Stem Cell Protocols Volume II: Differentiation Models (Methods in Molecular Biology) (Kurstad Turksen, Ed., 2006); Human Embryonic Stem Cell Protocols (Methods in Molecular Biology) (Kursad Turksen Ed., 2006); Mesenchymal Stem Cells: Methods and Protocols (Methods in Molecular Biology) (Darwin J. Prockop, Donald G. Phinney, and Bruce A. Bunnell Eds., 2008); Hematopoietic Stem Cell Protocols (Methods in Molecular Medicine) (Christopher A. Klug, and Craig T. Jordan Eds., 2001); Hematopoietic Stem Cell Protocols (Methods in Molecular Biology) (Kevin D. Bunting Ed., 2008) Neural Stem Cells: Methods and Protocols (Methods in Molecular Biology) (Leslie P. Weiner Ed., 2008).

Unless specific definitions are provided, the nomenclature utilized in connection with, and the laboratory procedures and techniques of, molecular biology, analytical chemistry, synthetic organic chemistry, and medicinal and pharmaceutical chemistry described herein are those well known and commonly used in the art. Standard techniques may be used for recombinant technology, molecular biological, microbiological, chemical syntheses, chemical analyses, pharmaceutical preparation, formulation, and delivery, and treatment of patients.

Unless the context requires otherwise, throughout the present specification and claims, the word “comprise” and variations thereof, such as, “comprises” and “comprising” are to be construed in an open, inclusive sense, that is, as “including, but not limited to”. By “consisting of” is meant including, and typically limited to, whatever follows the phrase “consisting of:” By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are required and may or may not be present depending upon whether or not they affect the activity or action of the listed elements.

In this specification and the appended claims, the singular forms “a,” “an” and “the” include plural references unless the content clearly dictates otherwise. As used herein, in particular embodiments, the terms “about” or “approximately” when preceding a numerical value indicates the value plus or minus a range of 5%, 6%, 7%, 8% or 9%. In other embodiments, the terms “about” or “approximately” when preceding a numerical value indicates the value plus or minus a range of 10%, 11%, 12%, 13% or 14%. In yet other embodiments, the terms “about” or “approximately” when preceding a numerical value indicates the value plus or minus a range of 15%, 16%, 17%, 18%, 19% or 20%.

Reference throughout this specification to “one embodiment” or “an embodiment” or “an aspect” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

### EXAMPLES

### Example 1

### Screening and Selection of Bk B2 Receptor Monoclonal Antibodies

This example describes screening of hybridoma supernatants containing antibodies generated by immunization against a BKB2R polypeptide, for the ability to activate p-GSK3β. Activation was assessed by immunoassay determination of GSK3β in lysates prepared from WI-38 human fibroblasts after 60 minutes of treatment with anti-BKB2R hybridoma supernatants, and in lysates prepared from 3T3 mouse fibroblast cells after 10 minutes of treatment with anti-BKB2R hybridoma supernatants.

Mice were immunized with BKB2R polypeptides (SEQ ID NOS:73 and 74) and hybridomas were isolated, using standard protocols. Fifty hybridomas were grown from fused splenocytes of animals immunized with the mouse sequence (SEQ ID NO:74) and 50 were also grown from fused splenocytes of animals immunized with the human sequence (SEQ ID NO:73). Antibodies from each hybridoma were added to wells of an ELISA plate that had been pre-coated with the BKB2R peptide to measure peptide binding.

Fifty hybridoma supernatants were screened for the presence of anti-BKB2R antibodies that were capable of stimulating phosphorylation of GSK-3β (Glycogen Synthase Kinase-3-beta) in both murine fibroblast 3T3 cells and WI-38 human fibroblast cells. Phosphorylation of GSK-3β is an indication of the deactivation of GSK-3β, through the activation of the BK B2 receptor by the antibodies.

Stimulation of 3T3 Cells.

Murine 3T3 cells were cultured in DMEM supplemented with 10% fetal bovine serum (FBS) and 1% penicillin/streptomycin (P/S). Forty-eight hours prior to stimulation, the cells were plated at 5×10⁴ cells/cm² on 12-well plates in one mL of culture medium with FBS (approximately 1.8×10⁵ cells/ml/well). Twelve to twenty-four hours prior to stimulation the culture medium was replaced with one mL of serum-free DMEM.

Reagents.

Platelet-derived growth factor (PDGF, Sigma P8147-1VL, 250 ng) was reconstituted in 4 mM HCl containing 0.1% BSA to obtain a solution containing PDGF at 5 pg/mL, which was further diluted in 4 mM HCL/0.1% BSA to obtain a stock solution containing PDGF at 1000 ng/mL. This stock was further diluted 1:10 (v/v) in serum-free medium to obtain a 100 ng/mL (“2×”) solution, which was then diluted 1:1 with samples to achieve a final sample treatment concentration of 50 ng/mL.

Lysis Buffer (“RIPA CLB”) contained 5 μl/mL protease inhibitor cocktail (“PIC”, Sigma, St. Louis, Mo.; catalogue number P8340), 2 mM NaVO₄, 20 mM Na₄P₂O₇ and 1 mM phenylmethylsulfonylfluoride (PMSF).

Samples.

Culture medium was removed from 3T3 cell cultures and replaced with 0.5 mL per well of fresh DMEM containing no added serum; care was taken not to disturb cell adherence to the culture wells. Positive control wells received 50 ng/mL PDGF in DMEM/FBS; negative control wells received DMEM/FBS alone. Test wells received 0.5 mL of hybridoma supernatants. After a ten-minute incubation at 37° C., the media were removed by aspiration and the adherent cells were gently rinsed with PBS and the plates held on ice.

Lysis.

0.5 ml of lysis buffer was added to each well and cells were lysed on ice for 30 minutes. A cell lifter was used to transfer the contents of each well to a microfuge tube. The supernatants were microcentrifuged for 15 minutes to remove insoluble material. The supernatants were then collected into fresh tubes and stored at −80° C.

ELISA.

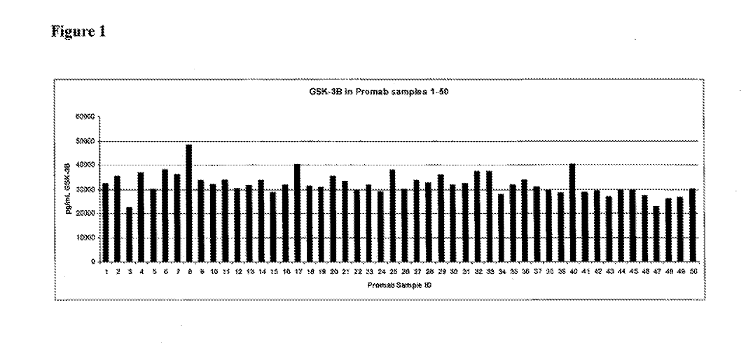

An immunoassay to quantify GSK-3β in the cell lysates was performed using the Assay Design® Kit (Assay Designs, Inc., Ann Arbor, Mich., Cat No. 900-123) according to the manufacturer's instructions. Samples and controls were diluted 1:50. The results are shown in FIG. 1. Two hybridoma clones (sample numbers 8 and 17) were selected for expansion, based on their high activity levels.

Stimulation of WI-38 Cells (Human).

Human WI-38 cells were cultured in MEM containing 10% FBS, 1% P/S and 2 mM L-glutamine. 48 hours prior to stimulation, the cells were plated at 5×10⁴ cells/cm² on 12 well plates (−1.8×10⁵ cells/mL/well) in 1 mL of culture media with FBS. 12-24 hours prior to stimulation, the medium was replaced with 1 mL of serum-free MEM.

Reagents.

PDGF was prepared as described above. Kallikrein (KLK, Sigma Cat. No. K3627) was dissolved in MEM containing 10% FBS and diluted to 200 μg/mL (2×); 500 μL of the KLK solution was added to selected culture wells to achieve a final concentration of 100 μg/mL. LiCl (Sigma L-8895) was dissolved in PBS and diluted to 40 mM in MEM/10% FBS; 500 μL of the LiCl solution was added to selected culture wells to achieve a final concentration of 20 mM. Lysis Buffer (RIPA CLB, from Assay Designs, Inc., MBL#061708C) was as described above.

Samples.

Culture medium was removed from WI-38 cell cultures and replaced with 0.5 mL per well of fresh DMEM containing no added serum; care was taken not to disturb cell adherence to the culture wells. Control wells received one of the following treatments: (A) 50 ng/mL PDGF in DMEM/FBS; (B) LiCl (20 mM), (C) KLK (500 μg/mL), (D) KLK (100 μg/mL), (E) negative control, DMEM/FBS alone, (F) negative control, serum-free DMEM. Test wells received 0.5 mL of hybridoma supernatants. After a sixty-minute incubation at 37° C., the media were removed by aspiration and the adherent cells were gently rinsed with PBS and the plates held on ice.

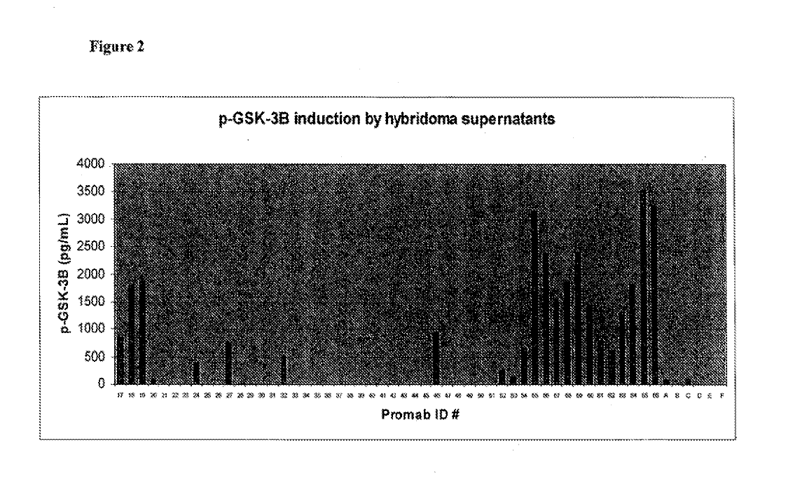

Lysis and ELISA immunoassay to quantify GSK-3β were as described above. The results are shown in FIG. 2. Multiple hybridoma supernatants induced GSK-3β significantly over background levels. Specifically, hybridoma supernatant sample numbers 55, 65 and 66 showed greater that 3000 pg/mL p-GSK-3B over background.

The anti-BKB2R antibody-containing hybridoma supernatants appear to have activated the BKB2R receptor, triggering inactivation (through phosphorylation) of GSK-3B.

### Example 2

### Acute Effects of Anti-Bkb2R Antibodies on Blood Pressure Using the Wistar Rat Model

This example describes the acute effects of several anti-BKB2R antibodies on blood pressure in anesthetized Wistar rats.

Study Design.

Male Wistar rats (Charles River Laboratories, Boston, Mass.) were 7.0 to 7.6 weeks old, weighed an average of 245 grams, and were maintained on Purina 5001 rat chow ad libitum. Following one week of laboratory acclimatization, treatments femoral catheter surgery and drug administration were conducted within a one-day period with measurements commencing the same day and continued during an ongoing three-week follow-up period. Treatment groups were (1) 3H3H3 (anti-BKB2R) antibody (n=8), (2) 3H3H9 (anti-BKB2R) antibody (n=8), (3) 1F2G7 (anti-BKB2R) antibody, (n=3), (4) 5F12G1 (anti-BKB2R) antibody (n=8).

Blood Pressure Measurements.

Rats were anesthetized with ketamine (30 mg/kg, IM) and Inactin (50 mg/kg, IP). Cannulae were implanted in the femoral artery for blood pressure measurements and in the femoral vein for drug administration. Arterial line was filled with saline with 10 UI/ml of heparin to keep the line patent over the experiment and avoid frequent flushing of the arterial line. After 15-20 minutes of equilibration period, and once the blood pressure was stable, a baseline blood pressure was recorded for 15 minutes; then, drugs were administered and its effects on blood pressure assessed. For the antibodies a single dose (0.5 mg/kg) was administered and blood pressure recorded for three hours. All drugs were diluted in saline or PBS to achieve a total volume of 1 ml/kg. Drugs were slowly administered, on a 40-sec period on average. Animals were kept at 37° C. during the experiment. At the end of the experiments, animals were euthanized and no blood or tissues were collected.

Calculations.

Baseline blood pressure, length of blood pressure response to drug, maximum blood pressure change and Area Under the Curve (AUC) for the blood pressure response. Blood pressure at 1, 2 and 3 h after infusion for the antibodies.

Results.

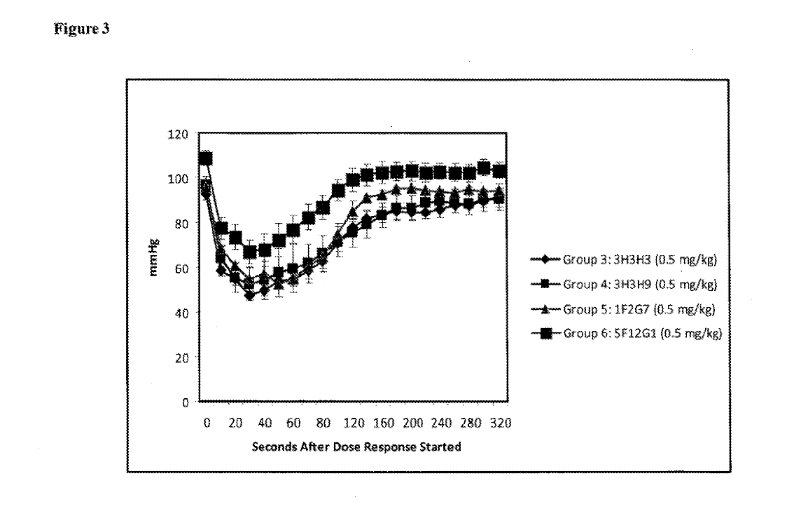

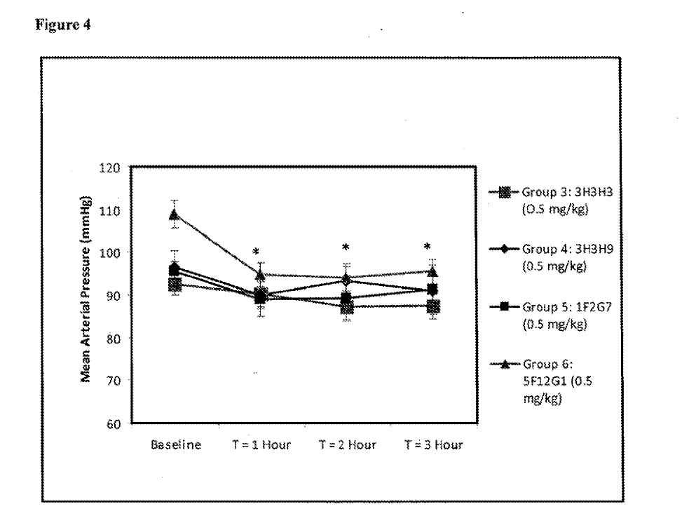

All four anti BKB2R antibodies had a transient effect on blood pressure starting immediately after IV administration. 5F12G1 showed a mild but significant reduction on blood pressure at all time points after administration. In this group, blood pressure at baseline was 109±3 mm Hg, and decreased to 95±3 mm Hg at one hour, to 94±3 mm Hg at two hours and 95±3 mm Hg at three hours after the antibody was administered. For these groups, the Peak Blood Pressure Response and the Length of the response (until blood pressure returned to baseline) values are presented in Table 1, and are shown in graph form in FIGS. 3 and 4.

TABLE 1
Blood Pressure Response and Length of Response

| | Length (sec) of Blood | Peak Blood Pressure |
|---|---|---|
| antibody | Pressure Response | Response (mm HG) |
| 3H3H3 | 193 +/− 23 | 41 +/− 1 |
| 3H3H9 | 171 +/− 28 | 45 +/− 5 |
| 1F2G7 | 147 +/− 18 | 45 +/− 1 |
| 5F12G1 | 168 +/− 29 | 57 +/− 4 |

### Example 3

### qRT-PCR Analysis of Viral Titer Reduction in A549 Cells by the Monoclonal Antibodies

Quantitative real-time polymerase chain reaction (qRT-PCR) methods have been used as a primary low throughput screen, as a confirmatory screen and for mechanism of action studies using influenza virus. This example describes use of a qRTPCR assay to measure the amount of viral genomic RNA in virally infected cells in the presence of a test compound, as a direct correlate to the number of replicated viral particles. The assay provides direct and reliable measurements that can also suggest mechanism of action. In conjunction with this assay, a 96-well low-throughput-sequencing of the isolated cDNAs for the quantitation of the virus population has been developed.

Experimental Design and Methods

A549 Cell Culture and Influenza Virus Infection.

A549 cells (ATCC CCL-185, ATCC, Manassas, Va.) were grown to ˜95% confluency in tissue culture plates. Cells were maintained and plated in DMEM supplemented with 10% FBS and 1% Pen/Strep/Glutamine (Invitrogen, Carlsbad, Calif.). 24 h after plating, antibodies 5F12G1 (“G1”), 1F2G7 (“G7”), 3H3H9 (“H9”), and 3H3H3 (“H3”), as well as the positive control drug Tamiflu® were added to the plates as dilutions in culture medium, after which the plates were returned to incubate at 37° C./5% CO₂ for 1 h. The cells were then infected or mock-infected with virus. Infection took place using 0.1 multiplicity of infection (MDIs) of influenza strain A/Brisbane/07 (H1N1). To infect cells, the growth medium was removed and cells were washed 3× with DPBS. The virus was diluted in DMEM-PSG (or just DMEM-PSG containing no virus was used for mock infections) and was added to cells. Fresh antibody preparations were added again, after which the plates were returned to incubate at 37° C./5% CO₂ for 1 h. The cells were then removed from the incubator, the infection medium was replaced with fresh medium containing the appropriate antibody, control drug, or mock dilution in OptiPro™ (Invitrogen, Carlsbad, Calif.) serum-free medium/2 μg/ml trypsin, and the cells were returned to the incubator. The cells were incubated in 37° C./5% CO₂, and harvested at 72 h post-infection for qRT-PCR analysis. As a negative control, uninfected cells were subjected to the same procedures. A control plate with the dosed antibodies only (no viral infection) was also analyzed to determine the extent of cytotoxicity of each antibody dose in A549 cells. The control plate was prepared as described above but no virus (medium mock infection) was added to the cells. Cell viability was determined after 72 h.

Analysis of DNA and RNA quantities from biological matrices (e.g., tissues, fluids, or excreta) was conducted using Qiagen extraction kits (Qiagen GmbH, Valencia, Calif.) as required by the matrix type. The concentration of extracted RNA samples was measured by optical density (A₂₆₀). cDNA sequences were quantified by real-time PCR using a TaqMan® assay (Invitrogen) with custom designed primers complementary to a 200 nt section of the influenza M segment. RNA samples were first transcribed into cDNA using the Invitrogen SuperScript™ Reverse Transcriptase per the supplier's instructions, and cDNA was quantified in the same manner as DNA above (qRT-PCR). For this analysis by qRT-PCR, duplicate samples were pooled and analyzed; positive, negative, and no-template controls were also run. A known amount of template (e.g., plasmid containing the influenza M gene) was used to generate a standard curve. A linear comparison was created by plotting Ct values against the known copy number of the template. This plot was then used to estimate the amount of cDNA in unknown samples. Statistical analysis was performed and graphed using Microsoft Excel.

Results.

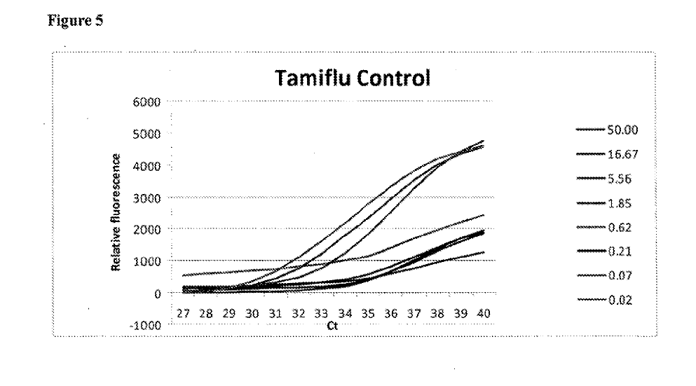

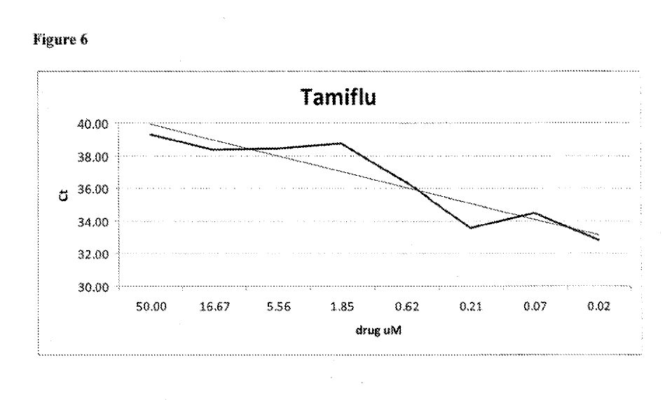

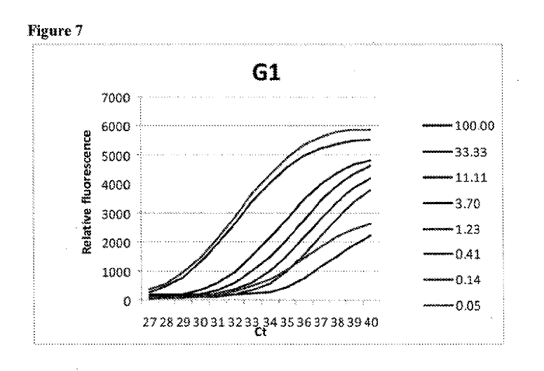

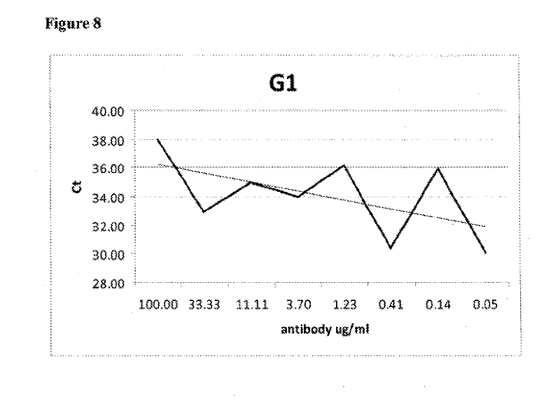

The results are summarized in table 2 and in FIGS. 5 to 8. The qRT-PCR assay results show that the Tamiflu® control reduced the measured number of viral genomic copies in a dose-responsive manner. Anti-BKB2R body G1 (5F12G1) showed a strong reduction in viral titer (reducing viral titer by 100-fold) at the highest tested concentration of 100 μg/ml, and was therefore considered a candidate for treatment of influenza virus.

TABLE 2
Ct values for qRT-PCR assay

| Concentration | | | | # of virus |
|---|---|---|---|---|
| (mAb in μg/mL) | Treatment | +/− Result FAM | Ct FAM | particles |
| 100.00 | G1 | Positive | 37.95 | 1560 |
| 33.33 | G1 | Positive | 32.97 | 50000 |
| 11.11 | G1 | Positive | 34.96 | 12500 |
| 3.70 | G1 | Positive | 34.02 | 12500 |
| 1.23 | G1 | Positive | 36.21 | 3120 |
| 0.41 | G1 | Positive | 30.38 | >100000 |
| 0.14 | G1 | Positive | 35.93 | 6250 |
| 0.05 | G1 | Positive | 30.08 | >100000 |
| 100.00 | G7 | Positive | 29.84 | >100000 |
| 33.33 | G7 | Positive | 28.85 | >100000 |
| 11.11 | G7 | Positive | 30.92 | >100000 |
| 3.70 | G7 | Positive | 30.52 | >100000 |
| 1.23 | G7 | Positive | 37.84 | 1560 |
| 0.41 | G7 | Positive | 29.09 | >100000 |
| 0.14 | G7 | Positive | 33.59 | 25000 |
| 0.05 | G7 | Positive | 32.82 | 50000 |
| 100.00 | H9 | Positive | 31.77 | 100000 |
| 33.33 | H9 | Positive | 28.52 | >100000 |
| 11.11 | H9 | Positive | 26.51 | >100000 |
| 3.70 | H9 | Positive | 29.47 | >100000 |
| 1.23 | H9 | Positive | 28.57 | >100000 |
| 0.41 | H9 | Positive | 32.30 | 50000 |
| 0.14 | H9 | Positive | 27.21 | >100000 |
| 0.05 | H9 | Positive | 25.81 | >100000 |
| 100.00 | H3 | Negative | 40.00 | 190 |
| 33.33 | H3 | Positive | 39.60 | 390 |
| 11.11 | H3 | Positive | 35.15 | 6250 |
| 3.70 | H3 | Positive | 30.13 | >100000 |
| 1.23 | H3 | Positive | 33.48 | 25000 |
| 0.41 | H3 | Positive | 34.69 | 12500 |
| 0.14 | H3 | Positive | 28.44 | >100000 |
| 0.05 | H3 | Positive | 36.16 | 3120 |
| 50.00 μM | Tamiflu ® | Negative | 39.33 | 390 |
| 16.67 μM | Tamiflu ® | Positive | 38.39 | 780 |
| 5.56 μM | Tamiflu ® | Positive | 38.44 | 780 |
| 1.85 μM | Tamiflu ® | Positive | 38.72 | 780 |
| 0.62 μM | Tamiflu ® | Positive | 36.38 | 3120 |
| 0.21 μM | Tamiflu ® | Positive | 33.57 | 25000 |
| 0.07 μM | Tamiflu ® | Positive | 34.51 | 12500 |
| 0.02 μM | Tamiflu ® | Positive | 32.83 | 50000 |
| G3 | 10⁵ particles | Positive | 31.53 | 100000 |
| Neg. | 0 particles | Negative | 40.00 | 0 |

### Example 4

### Monoclonal Anti-Bkb2R Antibodies Exhibit Cytotoxicity Against Mdck (Transformed) Cells

This example describes the effects of a monoclonal anti-BKB2R antibody on the Madin-Darby canine kidney (MDCK) cell line, an immortal, transformed renal epithelial cell line (Kushida et al., 1999). It was surprisingly observed that anti-BKB2R antibodies were cytotoxic to the MDCK cells, making these antibodies candidates for use as cancer therapeutics, such as for renal cancers.

Methods.

The antiviral and toxicity assay has been validated and was performed essentially as described in Noah et. al, Antiviral Res. 2007 January; 73 (1):50-9. Madin Darby canine kidney (MDCK) cells were used to test the efficacy of the anti-BKB2R monoclonal antibodies or other compounds in preventing the cytopathic effect (CPE) induced by influenza infection. Oseltamivir carboxylate (Tamiflu®) was included in each run as a positive control compound. Subconfluent cultures of MDCK cells were plated into 96-well plates for the analysis of cell viability (cytotoxicity). Antibodies or other drugs were added to the cells at the time of plating. 24 hours later, the CPE wells also received 100 tissue culture infectious doses (100 TCID₅₀s) of influenza virus. 72 hours later the cell viability was determined. Cell viability was assessed using Cell Titer-Glo™ (Promega, Madison, Wis.). The toxic concentrations of drug that reduced cell numbers by 50% and 90% (TC₅₀ and TC₉₀, respectively) were calculated.

CellTiter-Glo™ Detection Assay for Cell Viability.

Measurement of influenza-induced CPE was based on quantitation of ATP, an indicator of metabolically active cells. The CPE assay employed a commercially available CellTiter-Glo™ Luminescent Cell Viability Kit (Promega, Madison, Wis.) according to the supplier's instructions, to determine cytotoxicity and cell proliferation in culture. Briefly, following a cell culture incubation, the CellTiter-Glo™ Reagent was added directly to previously cultured, subconfluent cells in media, inducing cell lysis and the production of a bioluminescent signal (half-life greater than 5 hours, depending on the cell type) that was proportional to the amount of ATP present (as a biomarker for cell viability).

On day one, MDCK cells were grown to 90% confluency, then trypsinized, recovered, centrifuged, and washed twice in PBS to remove residual serum. Cells were resuspended and diluted in DMEM/pen/strep/L-glutamine, aliquoted into 96-well plates, and allowed to attach to the plate for 18 hours at 37° C. Antibodies (anti-BKB2R mAbs) or other test compounds, or vehicle (medium) controls, were then added to test wells.

On day two, a visual observation confirmed cell viability, with confluency visually estimated at 80-90%. 100 TCID₅₀s (100 times the tissue culture infectious dose that causes 50% lethality in 72 h) of each virus (containing 2 μg of trypsin, final concentration) was added to the test wells. Medium alone (also containing trypsin) was added to the control wells. Final well volumes were 100 mL. The plates were incubated for 72 h at 37° C./5% CO₂.

On day five, 100 ml of CellTiter-Glo™ reagent was added to each well, and plates were subsequently analyzed by luminescence detection.

Testing of Antibodies for Cytotoxicity in MDCK Cells.

On day one, MDCK cells recovered from 90% confluent monolayers were seeded cells in 96-well plates 18 hr prior to assay, at a cell density selected to achieve 90% confluency for uninfected cells on day two. Immediately after plating, test compounds (anti-BKB2R mAbs or Tamiflu®) diluted in culture medium containing less than 1% DMSO were added to replicate wells (triplicate for efficacy determinations, duplicate for cytotoxicity determinations); control wells received medium alone. Test compound (“drug”) preparations for the anti-BKB2R monoclonal antibodies (mAbs) had final concentrations of 100, 33, 11, 3.7, 1.2, 0.4, 0.14, 0.05 μg/ml; Tamiflu® preparations had final concentrations of 0.023, 0.07, 0.2, 0.6, 1.9, 5.5, 16.6, 50 mM. Cultures were maintained overnight at 37° C./5% CO₂ and on day two, virus was added. To each well in which efficacy determination was to be conducted, 100TCID₅₀s of virus (final test concentrations) were added; wells that did not receive virus were used for cytotoxicity determinations. The plates were incubated for an additional 72 h at 37° C./5% CO₂, after which cell viability was measured by luminescence analysis using the Promega CellTiter-Glo™ kit as described above.

Results.

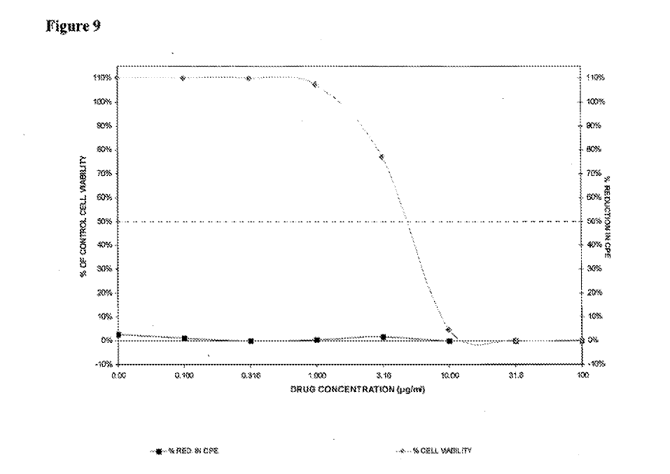

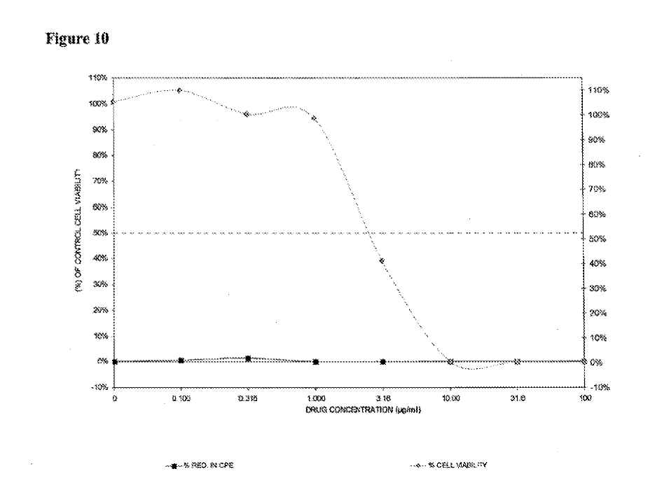

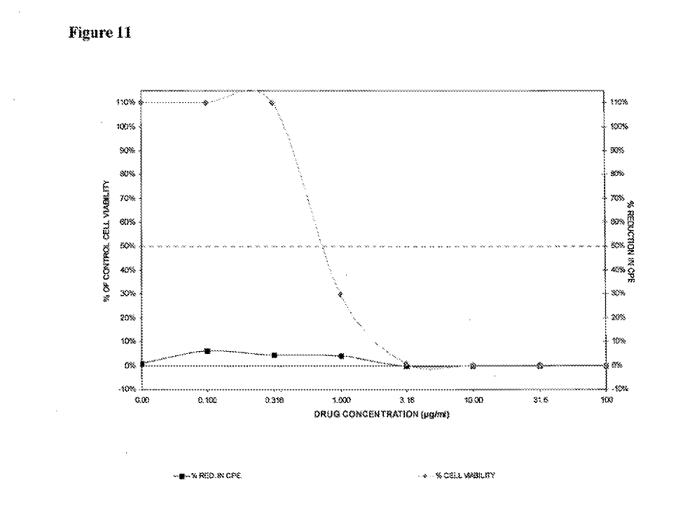

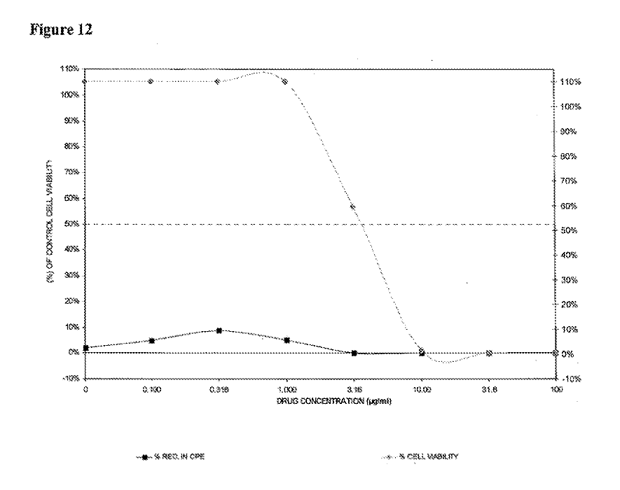

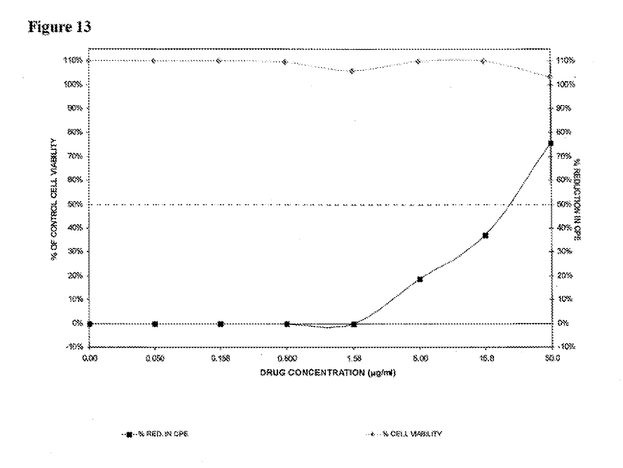

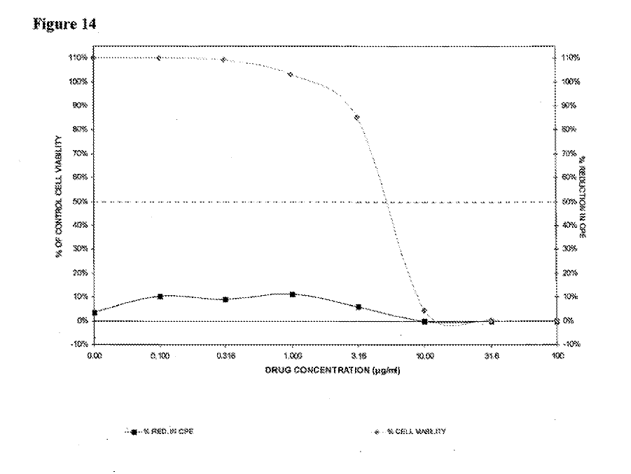

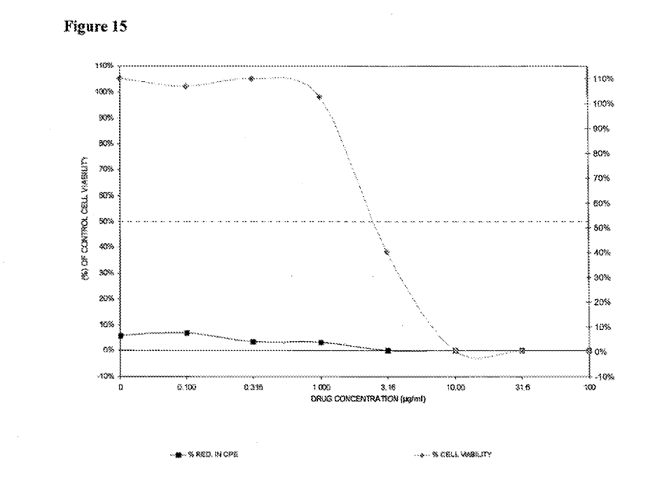

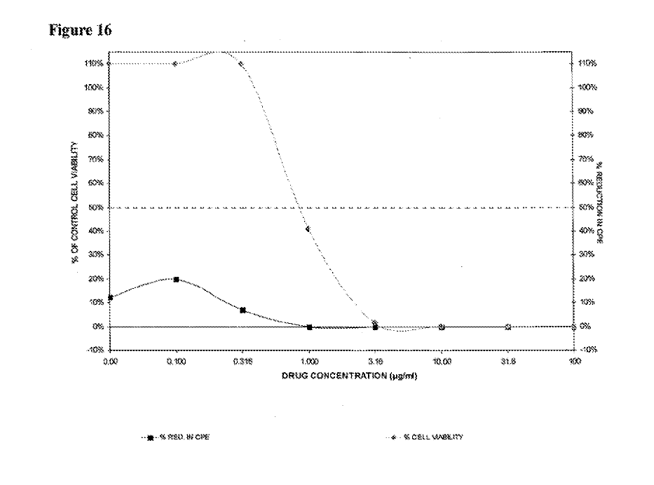

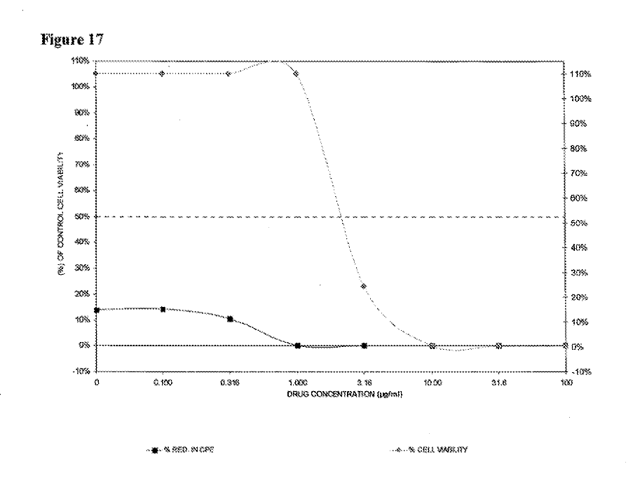

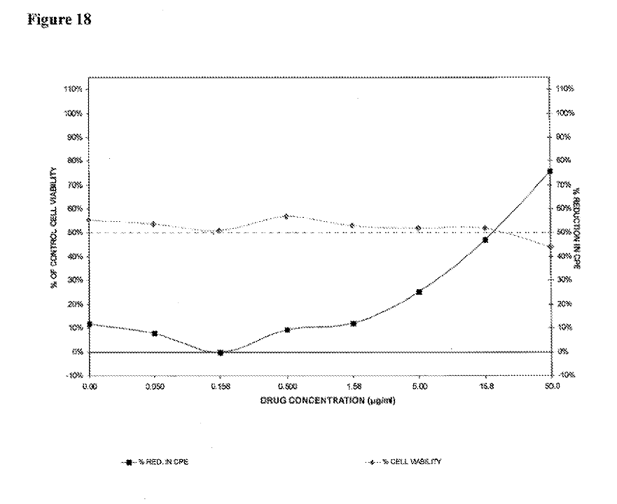

All of the tested anti-BKB2R antibodies showed cytotoxicity in MDCK cells at the higher concentrations tested. FIGS. 9-18 summarize, in graph form, the results, with the various antibodies, as compared to A/Brisbane/59/07 and Influenza CA/07/09.

### Example 5

### Anti-BKB2R Monoclonal Antibodies Exhibit Cytotoxicity Against a Variety of Cancer Cell Lines

This Example describes characterization of the cytotoxic activity of herein described anti-BKB2R monoclonal antibodies against a panel of cancer cell lines. BxPC-3 is a human adenocarcinoma cell line originally isolated from the pancreas (pancreatic cancer) (ATCC # CRL-1687; Tan et al., Cancer Invest. 4: 15-23, 1986. PubMed: 3754176). MV-4-11 is a human biphenotypic B myelomonocytic leukemia (mixed-lineage leukemia, MLL-AF4) cell line originally isolated from the peripheral blood (ATCC # CRL-959; Lange et al., Blood 70: 192-199, 1987. PubMed: 3496132). Hep G2 is a human hepatocellular carcinoma cell line isolated from the liver (liver cancer) (ATCC # HB-8065; Aden et al., Nature 282: 615-616, 1979. PubMed: 233137). RS4;11 is a human acute lymphoblastic leukemia (mixed-lineage leukemia, MLL-AF4) cell line isolated from the bone marrow (ATTC # CRL-1873) Stong et al., Blood 65: 21-31, 1985. PubMed: 3917311). HT-29 is a human colorectal adenocarcinoma (ATTC # HTB-38) cell line isolated from the colon (colon cancer). Fogh et al., J. Natl. Cancer Inst. 58: 209-214, 1977. PubMed: 833871). NUGC-4 is a human stomach carcinoma isolated from the stomach paragastiric lymph node (JCRB # JCRB0834; Akiyama et al., Jpn. J. Surg., 18: 438-446, 1988). PC-3 is a human prostate adenocarcinoma cell line originally isolated from bone metastasis (prostate cancer) (ATCC # CRL-1435; Kaighn et al., Invest. Urol. 17: 16-23, 1979. PubMed: 447482).

Testing of Anti-BKB2R Monoclonal Antibodies (1F12G7 and 5F12G1) for Cytotoxicity in Cancer Cell Lines BxPC-3, MV-4-11, Hep G2, RS4;11, HT-29 and NUGC-4.

Cell lines were grown using media, serum, and culture conditions recommended by the ATCC guidelines for each cell line (ATCC, Manassas, Va.). Cells were seeded into 96-well culture plates at 30,000 cell/well on day 0 in a volume of 0.1 mL complete medium. Plates were then placed in a humidified incubator at 37° C. with 5% CO₂ and 95% HEPA filtered room air for 24 hrs. Next, 0.1 mL of serum-free medium in which was diluted each test antibody at twice (2×) the desired final concentration (50,000 ng/ml, 25,000 ng/ml, 12,500 ng/ml 6,250 ng/ml, 3125 ng/ml, 1563 ng/ml, 781 ng/nl, 391 ng/ml, 195 ng/ml, or 98 ng/ml) was added to indicated wells and the plates were returned to the incubator for 120 hours (5 days). A positive control, 0.1 mL of a 2× concentration of the anti-cancer drug cisplatin, was used at the following concentrations: 300,050.000 ng/ml, 75,012.500 ng/ml, 18,753.125 ng/ml, 4,688.281 ng/ml, 1,172.070 ng/ml, 293.018 ng/ml, 73.254 ng/ml, 18.314 ng/ml, 4.578 ng/ml or 1.145 ng/ml.

MTT Assay.

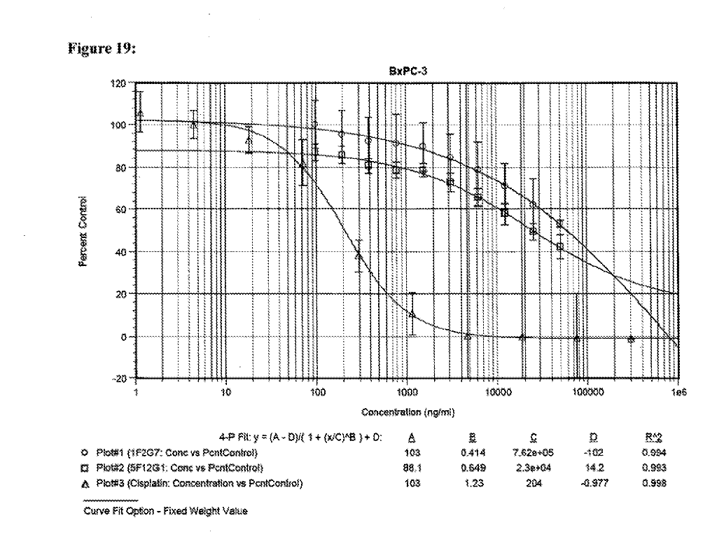

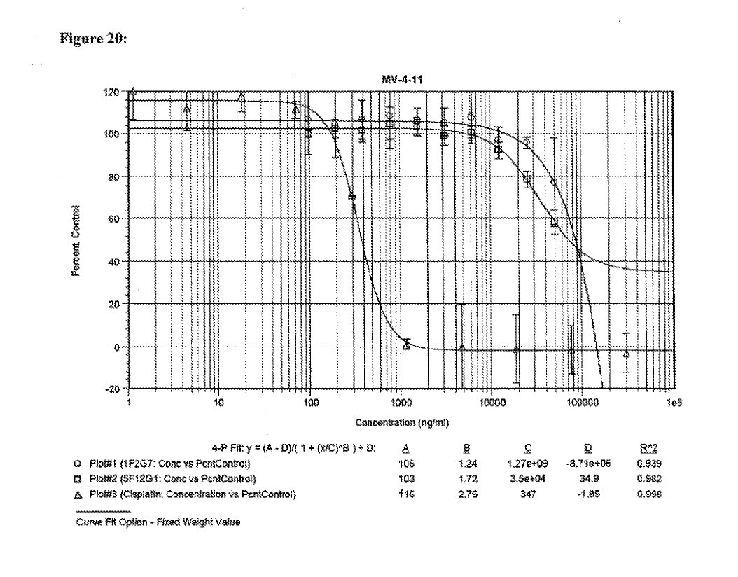

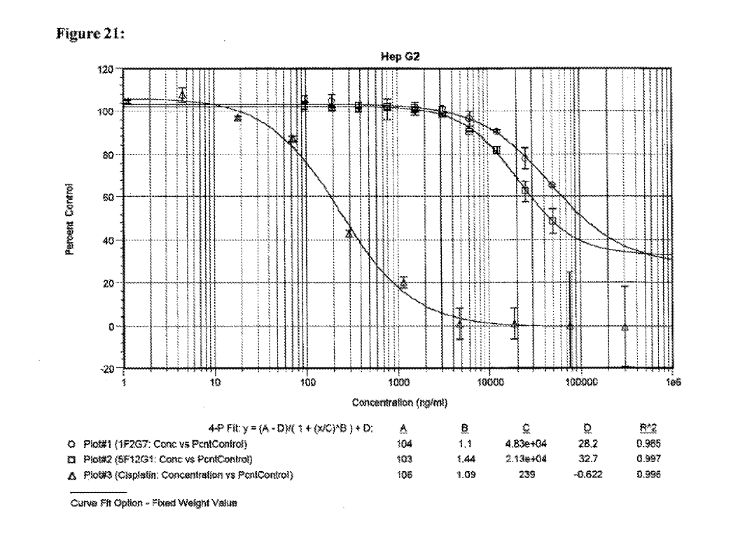

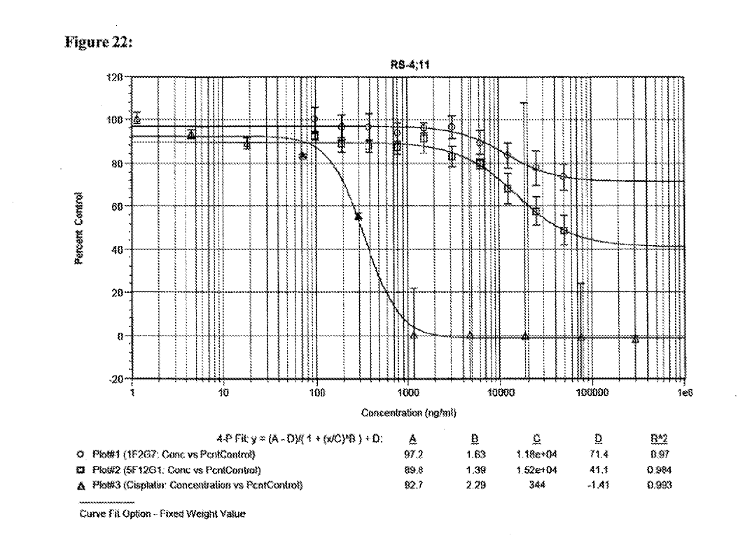

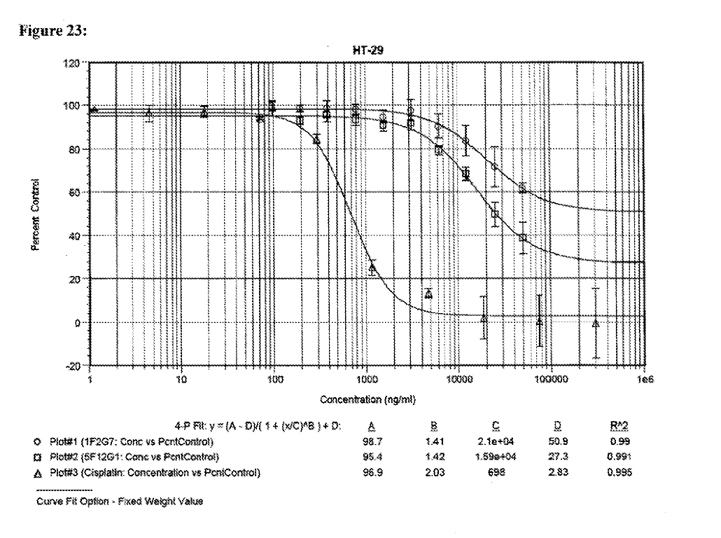

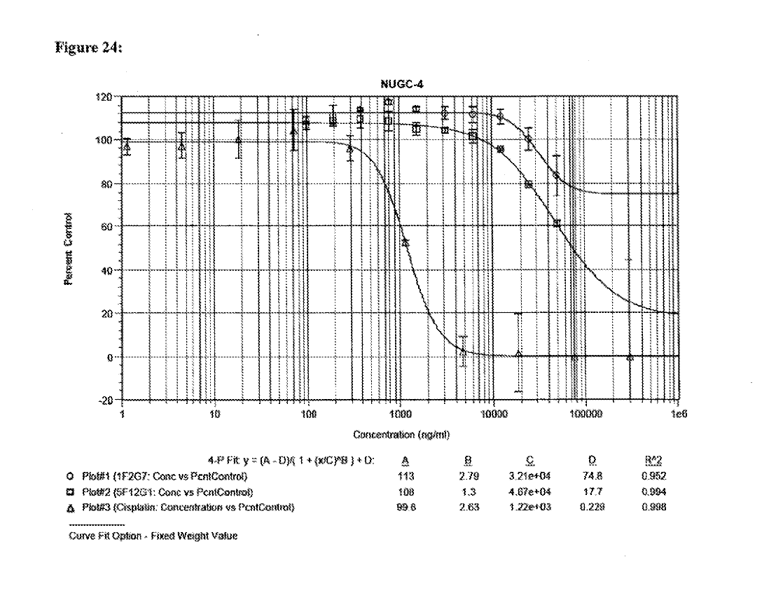

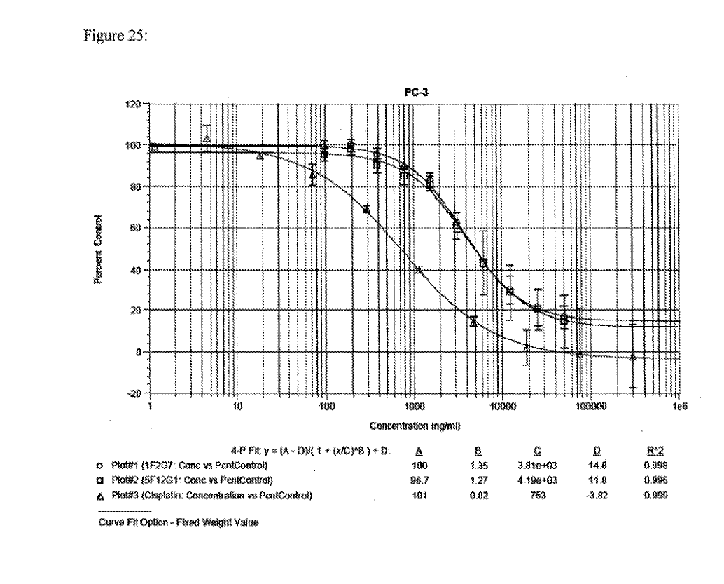

The anti-proliferative activity of test compounds against the indicated cell lines was evaluated in vitro using the ATCC's MTT Cell Proliferation Assay (Catalog No. 30-1010K). After the 120 hour incubation with drug (e.g., anti-BKB2R mAb or cisplatin), cell proliferation was measured by addition of MTT reagent to each well and incubation for an additional 4 hrs. This step was then followed by addition of the cell lysis/MTT solublization reagent and incubation overnight. Optical absorbance (570 nm) of the test wells was measured and then quantitated relative to control wells that received no drug. Results were expressed as percent inhibition versus compound concentration and graphed, as shown in FIGS. 19-25, for cell lines BxPC-3, MV-4;11, HepG2, RS-4;11, HT-29, NUGC-4, and PC-3, respectively. Based on these results, the EC₅₀ concentration for each antibody, in each cell line, was calculated and tabulated in comparison to the cisplatin-treated control, in Table 3, below. Both anti-BKB2R mAbs tested showed marked cytotoxicity toward all tested cancer cell lines following 120 hours of exposure.

TABLE 3
Cytotoxicity of anti-BKB2R mAbs toward cancer cell lines

| | EC 50 Values (ng/ml) | | | | | | |
|---|---|---|---|---|---|---|---|
| | BxPC-3 | Hep G2 | HT-29 | NUGC-4 | PC-3 | MV-4-11 | RS-4;11 |
| 1F2G7 | 6.2E+04 | 4.8E+04 | 2.1E+04 | 3.2E+04 | 3.8E+03 | >5.0E+04 | 1.2E+04 |
| 5F12G1 | 2.3E+04 | 2.1E+04 | 1.6E+04 | 4.7E+04 | 4.2E+03 | 3.5E+04 | 1.5E+04 |
| Cisplatin | 204 (0.7 μM) | 239 (0.8 μM) | 698 (2.3 μM) | 1.2E+03 | 753 (2.5 μM) | 347 (1.2 μM) | 344 (1.1 μM) |
| | | | | (4.1 μM) | | | |

### Example 6

### Bradykinin Receptor Agonist Monoclonal Antibody 5F12G1 Increases Insulin Sensitivity

The hyperinsulinemic euglycemic clamp has been considered to as the standard in vivo technique for measuring insulin sensitivity effects of type 2 diabetes drugs. In this procedure, insulin is administered to a test animal to raise the insulin concentration, while glucose is infused to maintain euglycemia. The glucose infusion rate (GIR) needed to maintain euglycemia is a reflection of insulin action or improved insulin sensitivity. The bradykinin receptor agonist (anti-BKB2R) monoclonal antibody clone 5F12G1 was tested in a euglycemic clamp study to measure its ability to improve insulin sensitivity.

Materials and Methods.

Healthy young male Sprague Dawley Rats weighing 275-300 g were used for the study (Harlan Laboratory, Indianapolis, USA). The rats were maintained in a controlled environment at a temperature of 70-72° F., humidity 30-70%, with a photo cycle of 12 hours of light and 12 hours of dark. They were provided with TEKLAD™ 2018-Global 18% diet and drinking water ad libitum. After seven days of acclimatization, rats were grouped in groups of four.

Hyperinsulinemic-Euglycemic Clamp.

Animals were anesthetized with an intraperitoneal injection of ketamine-plus-xylazine cocktail and the right jugular vein and left carotid artery were catheterized externally through an incision in the skin flap. The catheterized animals were allowed to recover for five days. After five days of recovery, animals were fasted for six hours and a 120-minute hyperinsulimic-euglycemic clamp was applied with continuous infusion of human insulin (Humulin, Eli Lilly, Indianapolis, Ind.) at a constant rate of 4mU/kg/minute. At the same time a 20% glucose solution at variable rate was infused and the rate was adjusted every 10 minutes to maintain a target blood glucose level of 115±5 mg/dl. Both insulin and glucose were infused through catheterized right jugular vein and blood glucose levels were monitored from the catheterized carotid artery. Arterial blood glucose levels and plasma insulin levels were measured prior to infusion at t=−120, −90, −30, −15 and 0 minutes and then at every 10 minutes for 120 minutes (t=120), using a Glucose meter (Accu-Chek™ Roche Diagnostics, Indianapolis, Ind.) and a rat insulin ELISA kit. The clamps were continued for 120 minutes (t=120), after which the experiment was terminated. The vehicle group was injected with PBS (i.m.) at t=−30 min and the 5F12G1 treated group was injected with the antibody (i.m.) at t=−30 min at 0.5 mg/kg concentration.

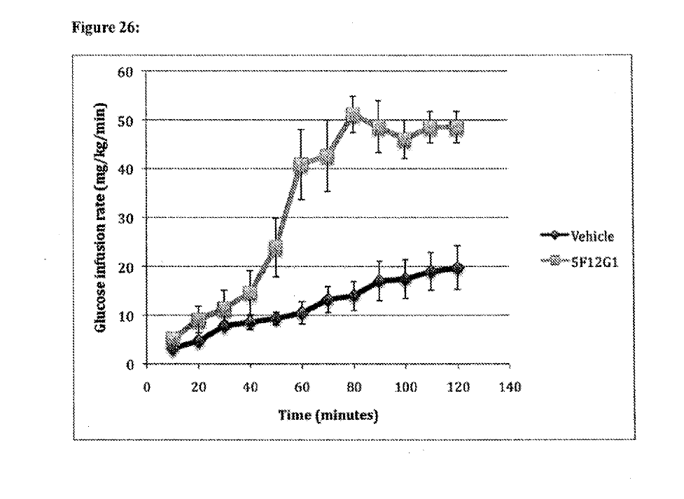

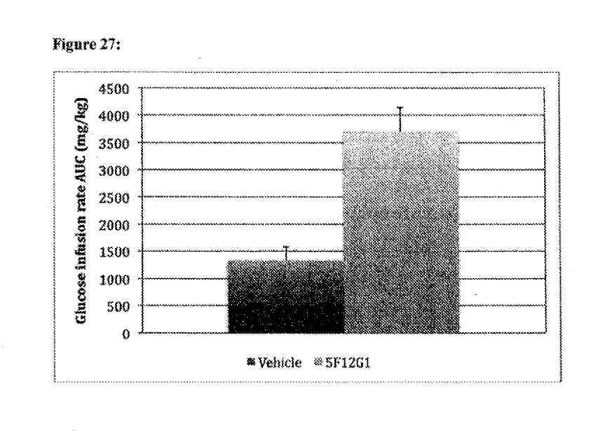

The glucose infusion rate increased significantly upon treatment with antibody 5F12G1 with a peak increase of 291% compared to vehicle (t=60 min) (p=0.0066) and a 179% increase in total glucose infusion rate AUC compared to vehicle (p=0.0035). These results demonstrated the ability of 5F12G1 to significantly increase insulin sensitivity by improving the action of insulin. The results were tabulated and the glucose infusion rate was graphed as a function of time (FIG. 26), and as area under the curve (AUC) (Table 4 and FIG. 27).

TABLE 4
Calculated Glucose Infusion Rate Area Under the Curve (mg/kg)

| Animal # | Vehicle | 5F12G1 |
|---|---|---|
| 1 | 1193 | 4560 |
| 2 | 1746 | 4317 |
| 3 | 1693 | 3243 |
| 4 | 673 | 2685 |

### Example 7

### Effects of 5F12G1 ON OGTT in Zucker Diabetic Fatty Rats

This Example describes evaluation of oral glucose tolerance in Zucker Diabetic fatty (ZDF fa/fa) rats treated with 5F12G1 monoclonal antibody. Male ZDF fa/fa rats (Charles River) were maintained on a Harlan Tekled diet with Arrowhead drinking water ad libitum and allowed to acclimatize for one week. Six animals per group were treated according to the following treatment groups: 1, sterile PBS (vehicle control); 2, 1.0 mg/kg murine monoclonal antibody (mAb) 5F12G1 (VH comprising SEQ ID NO:1, VL comprising SEQ ID NO:2); 3, 0.2 mg/kg mAb 5F12G1; 4, 0.04 mg/kg mAb 5F12G1.

Oral Glucose Tolerance Test. The Oral Glucose Tolerance Test (OGTT) was performed on overnight fasted (16 hours) rats. Vehicle control (PBS) or 5F12G1 monoclonal antibody was administered subcutaneously thirty minutes prior to glucose loading. D-glucose was prepared in distilled water and administered orally at 2 g/kg body weight.

At multiple time points (0, 15, 30, 60, 90 and 120 minutes) blood samples of approximately 50 μl each were collected and processed to isolate the plasma. The plasma samples were analyzed for insulin by an ELISA method using an ultra sensitive mouse insulin ELISA kit (Crystal Chem, Inc., Downers Grove, Ill.). ELISA data were compiled and used to calculate the mean±standard error (SEM) with Microsoft Excel or GraphPad Prism version 5.00 for Windows (GraphPad Software, San Diego Calif. USA).

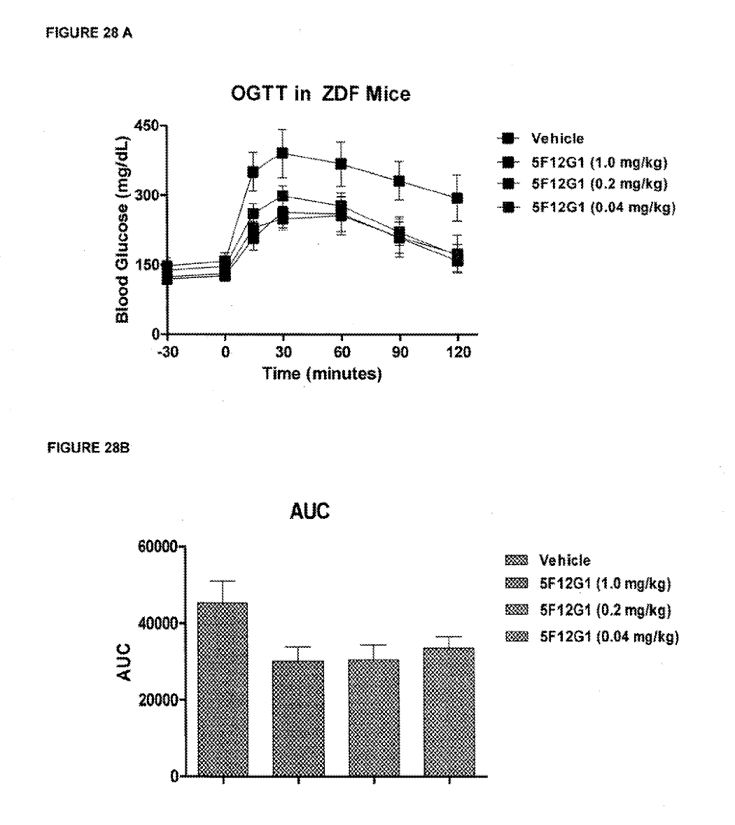

Results. The results are presented in FIGS. 28A, 28B, 29A, and 29B. Compared to treatment with the vehicle control, single administration of monoclonal antibody 5F12G1 (1.0, 0.2 and 0.04 mg/kg) decreased the area under curve (AUC) of blood glucose concentration after oral loading of glucose in DIO rats. The decrease in AUC of blood glucose was higher with 1.0 mg/kg followed by 0.2 and 0.04 mg/kg body weight. Monoclonal antibody, 5F12G1 dose dependently increased insulin activity in OGTT ZDF fa/fa rats.

### Example 8

### Effects of 5F12G1 on OGRR in DIO Mice

This Example describes evaluation of oral glucose tolerance in diet induced obese (DIO) mice treated with the anti-BKB2R monoclonal antibody 5F12G1 (VH comprising SEQ ID NO:1, VL comprising SEQ ID NO:2). Male C57BL/6J mice (Jackson Laboratory, Bar Harbor, Me.) were maintained on a 60 kcal % fat diet with Research Diet and Arrowhead drinking water ad libitum and permitted to acclimatize for a period of eight weeks. Ten animals per group were treated according to the following treatment groups: 1, sterile PBS (vehicle control); 2, 1.0 mg/kg murine monoclonal antibody (mAb) 5F12G1; 3, 0.2 mg/kg mAb 5F12G1; 4, 0.04 mg/kg mAb 5F12G1.

Oral Glucose Tolerance Test. The Oral Glucose Tolerance Test (OGTT) was performed on overnight fasted (16 hours) mice. Vehicle control (PBS) or 5F12G1 monoclonal antibody was administered subcutaneously thirty minutes prior to glucose loading. D-glucose was prepared in distilled water and administered orally at 2 g/kg body weight. Blood glucose levels were measured before administration of vehicle or 5F12G1 (−30 minutes) and just before glucose loading (0 minute) and at ensuing timepoints of 15, 30, 60 90 and 120 minutes using an Accu-Chek™ glucose meter (Roche Diagnostics, Indianapolis, Ind.) according to the manufacturer's instructions.

At multiple time points (0, 15, 30, 60, 90 and 120 minutes) blood samples of approximately 50 μl each were collected and processed to isolate the plasma. The plasma samples were analyzed for insulin by an ELISA method using an ultra sensitive mouse insulin ELISA kit (Crystal Chem, Inc., Downers Grove, Ill.). ELISA data were compiled and used to calculate the mean±standard error (SEM) with Microsoft Excel or GraphPad Prism version 5.00 for Windows (GraphPad Software, San Diego Calif. USA).

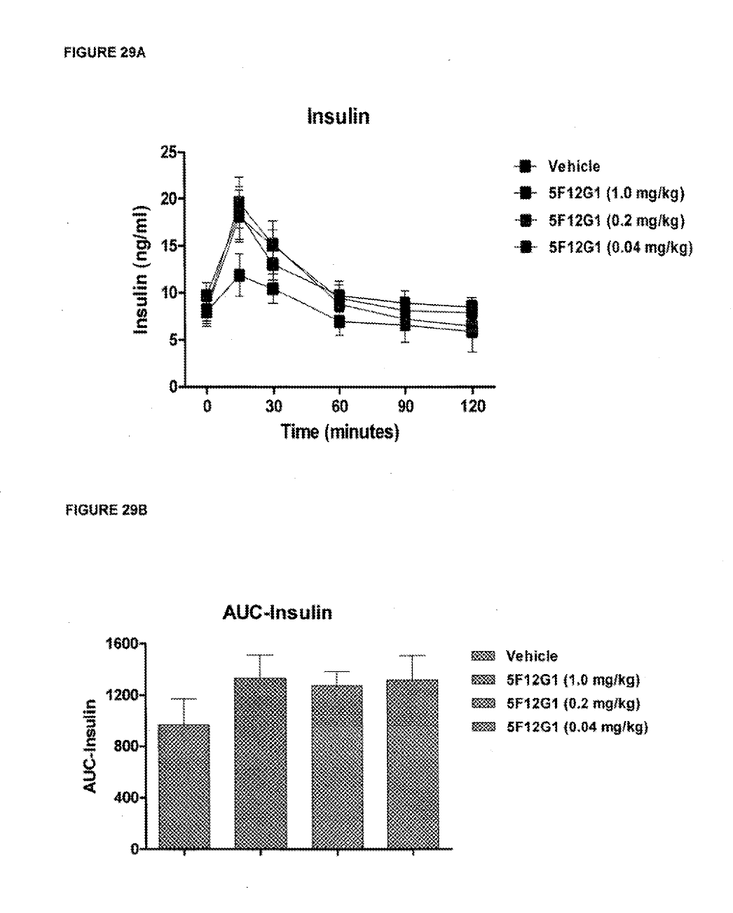

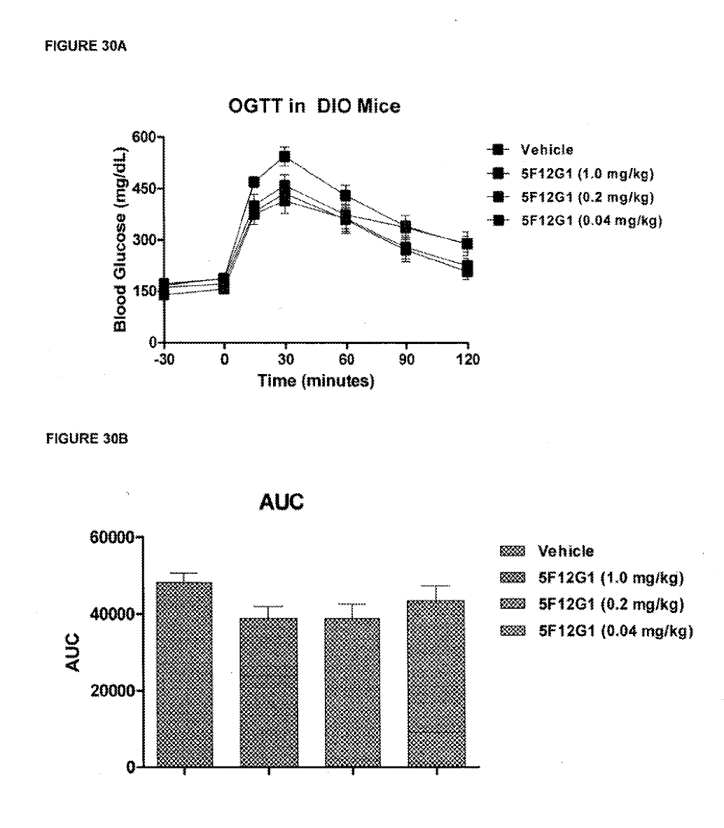

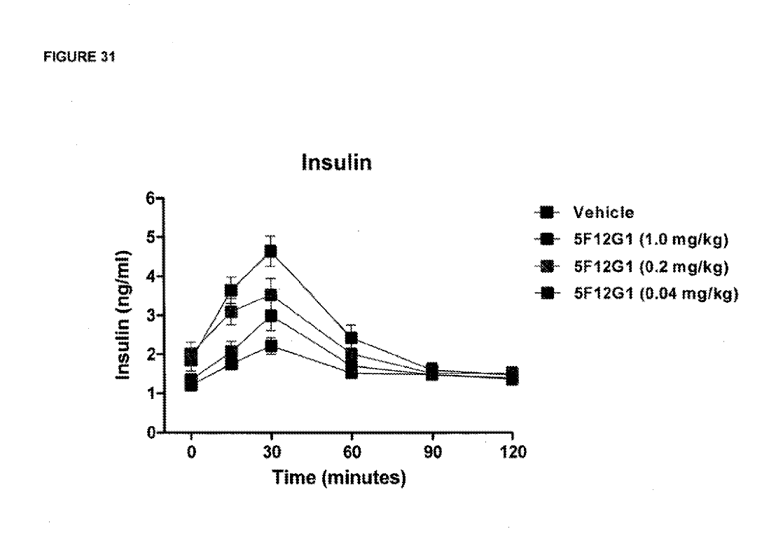

Results. The results are presented in FIGS. 30A, 30B and 31. Compared to treatment with the vehicle control, single administration of monoclonal antibody 5F12G1 (1.0, 0.2 and 0.04 mg/kg) decreased the area under curve (AUC) of blood glucose concentration after oral loading of glucose in DIO mice. The decrease in AUC of blood glucose was higher with 1.0 mg/kg followed by 0.2 and 0.04 mg/kg body weight. Monoclonal antibody, 5F12G1 dose dependently increased insulin activity in OGTT DIO mice.

### Example 9

### 5F12G1 is an Agonist of the Human Bradykinin B2 Receptor

This example describes testing of the dose-dependent stimulatory response of the monoclonal anti-BKB2R antibody 5F12G1 on the bradykinin receptor B2 as measured by downstream intracellular calcium release. A stable CHO cell line expressing the human BKB2 receptor (CHO-K1/B2/Gα15) was used for the screening. The antibody was diluted to five different concentrations, from 0.5 mg/ml, via three-fold dilution increments, and screened on duplicate cell samples.

Expression and functional activity of the human BKB2 receptor in the CHO-K1/B2/Gα15 cell line were validated by exposure to the positive control, bradykinin. The EC₅₀ value was similar to the reported values for bradykinin. The stimulatory activity of the 512G1 antibody was normalized to the positive control; data were compiled as % activation.

To perform the assay, CHO-K1/B2/Gα15 cells were seeded in wells of a 384-well black-wall, clear-bottom plate at a density of 20,000 cells per well in 20 μL of growth medium 20 hours prior to the day of experiment, and maintained at 37° C./5% CO₂. 20 μL of dye-loading solution (FLIPR™ Calcium 4 assay kit, Molecular Devices, Sunnyvale, Calif.) was added into each well and the plate was placed into a 37° C. incubator for 60 minutes, followed by 15 minutes at room temperature. The total reading time was 120 sec. After a 20-second reading to establish the baseline, the antibody or agonist were added to selected wells and the fluorescence signal was captured for another 100 seconds (21 s to 120 s). Readings from wells containing cells stimulated with assay buffer (0.03% Na₃N PBS) containing 1% DMSO were chosen as the background values for screening; readings from wells containing cells stimulated with the agonist bradykinin (at 10 uM) were chosen as the positive control.

Results. For cells treated with mAb, 5F12G1, the percentage of activation was 72.7+/−3.5% (mean+/−SD, n=2) at 0.5 mg/ml, and the ED₅₀ was 0.24 mg/ml. For cells treated with bradykinin, the percentage of activation was 93.1+/−5.7% (mean+/−SD, n=2), and the ED₅₀ was 0.95 nm/l. Exposure to the monoclonal antibody 5F12G1 thus resulted in a high % activation of cells expressing the human bradykinin receptor B2.

### Example 10

### Effects of 5F12G1 Antibody Administration in Chronic Type 2 Diabetes

This example describes 21-day evaluation of the effects of the anti-BKB2R mAb 5F12G1 at three different doses in the chronic Type II diabetes model of ZDF fa/fa rats, as compared to exenatide, sitagliptin and mAb MG2b-57.

The ZDF fa/fa rat is a model for Type 2 diabetes based on impaired glucose tolerance caused by the inherited obesity gene mutation that leads to insulin resistance. In ZDF fa/fa rats, hyperglycemia is initially manifested at about seven weeks of age, and obese male rats are fully diabetic by approximately 12 weeks. Between seven and ten weeks of age, blood insulin levels in theses animals are elevated (hyperinsulinemia), but the insulin levels subsequently drop as the pancreatic beta cells cease to respond to the glucose stimulus.

The fasting hyperglycemia, which first appears at 10 to 12 weeks of age, progresses with aging; insulin resistance and abnormal glucose tolerance become progressively worse with age. Left untreated, the ZDF rats eventually exhibit hyperlipidemia, hypertriglyceridemia and hypercholesterolemia, resulting in mild hypertension.

Test compounds and vehicle used in this study were: 1. mouse monoclonal anti-BKB2R antibody 5F12G1 (IgG2b,κ); 2. mouse monoclonal antibody MG2b-57 (BioLegend, San Diego, Calif.), chosen as an isotype-matched control (IgG2b,κ) for 5F12G1 and having an irrelevant antigen specificity (e.g., negative control); 3. sitagliptin (Selleck Chemicals LLC, Houston, Tex.); 4. exenatide (Bachem Americas, Torrance, Calif.).

Sitagliptin (Januvia®) is an antihyperglycemic (antidiabetic drug) of the dipeptidyl peptidase-4 (DPP-4) inhibitor class. Sitagliptin works to competitively inhibit the enzyme dipeptidyl peptidase 4 (DPP-4), which breaks down the incretins GLP-1 and GIP, gastrointestinal hormones released in response to a meal. By preventing GLP-1 and GIP inactivation, they are able to increase the secretion of insulin and suppress the release of glucagon by the pancreas. This effect drives blood glucose levels towards normal.

Exenatide is a 39-amino-acid peptide, an insulin secretagogue, with glucoregulatory effects. Exenatide is a synthetic version of exendin-4, a hormone that displays biological properties similar to human glucagon-like peptide-1 (GLP-1), a regulator of glucose metabolism and insulin secretion. Exenatide enhances glucose-dependent insulin secretion by the pancreatic beta-cell, suppresses inappropriately elevated glucagon secretion, and slows gastric emptying.

Animals. Male ZDF fa/fa rats were obtained from CRL (Kingston, N.Y.). Upon arrival, rats were seven weeks of age. The rats were housed individually per cage in a room with a photo cycle of 12 hours of light and 12 hours of dark and an ambient temperature of 70-72° F. and fed on regular rodent diet and water ad libitum. At the age of eleven weeks, rats were divided into six groups (Table 5) of eight rats per group based on fasting blood glucose levels. A sub-group of four rats per group was maintained in parallel to the main groups and was dosed similarly for twenty-one days for a hyperinsulinemic-euglycemic clamp study.

TABLE 5
ZDF fa/fa rat groups

| | | N | N | | | | | |
|---|---|---|---|---|---|---|---|---|
| | | (Main | (Sub- | | | | Dosing | Dosing |
| Group | Description | group) | group) | ROA | Dose | | Volume | Frequency |
| 1 | 5F12G1 | 8 | 4 | s.c. | 0.2 | mg/kg | 200 μl/rat | Every 3 days |
| 2 | 5F12G1 | 8 | 4 | s.c. | 0.04 | mg/kg | 200 μl/rat | Every 3 days |
| 3 | 5G12G1 | 8 | 4 | s.c. | 0.008 | mg/kg | 200 μl/rat | Every 3 days |
| 4 | Negative Control MG2b-57 | 8 | 4 | s.c. | 0.2 | mg/kg | 200 μl/rat | Every 3 days |
| 5 | Sitagliptin | 8 | 4 | p.o. | 10 | mg/kg | 500 μl/rat | 1×/daily |
| 6 | Exenatide | 8 | 4 | i.p. | 1 | μg/kg | 200 μl/rat | 2×/daily |

The test compounds 5F12G1 and MG2b-57 were administered subcutaneously once every three days. Exenatide was administrated intraperitoneally twice every day and sitagliptin was administrated orally once every day for a period of twenty-one days. 5F12G1 was administrated at three different doses, 0.2, 0.4 and 0.008 mg/kg, exenatide at 1 μg/kg, sitagliptin at 10 mg/kg and MG2b-57 at 0.2 mg/kg, respectively.

An oral glucose tolerance test (OGTT) was performed on Day 0, 7, 14 and 21 for each group of the study. Plasma samples were collected at each time point during OGTT to also measure insulin levels. Body weight, food and water intake were measured twice a week. Blood pressure and heart rates were monitored on Day 0, 7, 14 and 21 using a non-invasive tail cuff method (with five readings per rat taken and then averaged). Fasting serum samples were collected on Day 0, 7, 14 and 21 before OGTT for determination of triglyceride and total cholesterol level. Urine samples were collected on Day 7 and 14 for the determination of glycosuria. Glycated (Glycosylated) hemoglobin (HbA1c) was measured at the end of the study. Assay kits for these studies were as presented in Table 6, and were used according to the suppliers' instructions.

TABLE 6
Assay Kits

| TEST | TEST KIT | VENDOR |
|---|---|---|
| Glucose | Accu Check Glucose | Roche, CA |
| (For OGTT) | Meter | |
| Triglyceride | Triglycerides kit | Wako Chemicals |
| | | USA, Inc. Richmond, |
| | | VA |
| Cholesterol | Cholesterol kit | Wako Chemicals |
| | | USA, Inc. Richmond, |
| | | VA |
| Insulin | Ultra Sensitive Rat | ALPCO Diagnostic, |
| | Insulin ELISA kit | Inc. |
| Glycated hemoglobin, | Bayer A1C Now+ | Bayer Healthcare, US |
| HbA1c | | |
| Glycosuria | Glucose Auto kit | Wako Chemicals |
| | | USA, Inc. Richmond, |
| | | VA |

Oral Glucose Tolerance Test (OGTT)

Because of the rats' age at the start of this study, the ZDF fa/fa rats were expected to have slight insulin resistance resulting in higher than normal increase in blood glucose levels during an OGTT. Insulin resistance in the rats was expected to increase during the 21 day study as the animals aged, resulting in higher blood glucose levels in subsequent OGTT's.

OGTTs were performed on Day 0, 7, 14 and Day 21. Rats were fasted overnight and fasted blood glucose levels were measured (t=0 min), and then each rat was given a single 1.5 ml dose of glucose solution (2 g/kg body weight of D-(+)-glucose (G7528, Sigma) solubilized in deionized water) administered by oral gavage. The blood glucose levels were then measured by glucose meter at 15, 30, 60, 90 and 120 minutes to observe the rate of glucose clearance from the blood over time. At each time point of an OGGT approximately 50-60 μl of blood were collected and processed for plasma to measure insulin levels.

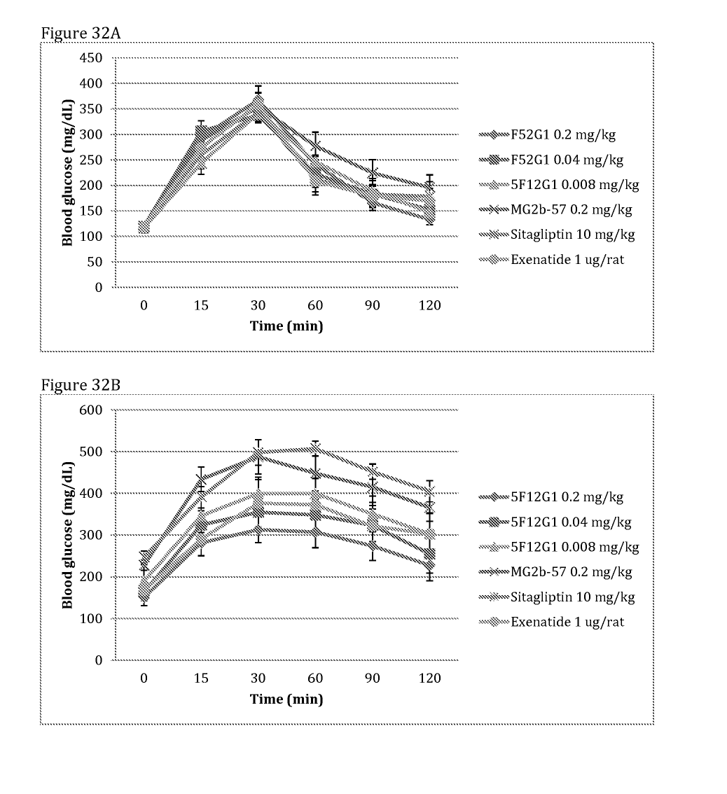

On Day-0, as expected, all the groups showed a similar pattern of glycemic response to the OGTT, in that blood glucose levels increased from about 100 mg/dl at time 0, and peaked at about 340-370 mg/dl at t=30 minutes, then gradually returned to baseline over the next 60 to 90 minutes (see FIG. 32A). At day 7, 14 and 21, rats in the negative control group, MG2b-57, exhibited progressively higher fasting blood glucose levels, higher peak blood glucose levels, and the glucose levels were elevated for increasingly prolonged periods of time during the OGTT. This result is expected as the ZDF rats develop type 2 diabetes and glycemic control is progressively lost. After 21 days of treatment, rats in the negative control group, MG2b-57, and animals in the sitagliptin treatment group had significantly higher fasting blood glucose levels (228 mg/dl) at the start of the OGTT and the blood glucose levels rose to 488 mg/dl at 30 minutes and remained high (FIG. 32B). This increase in blood glucose levels during an OGTT indicated the ZDF fa/fa rats were developing type 2 diabetes, as expected. However, rats treated with 5F12G1 had significantly lowered blood glucose levels at the start of the OGTT (150+/−20 mg/dl for the 0.2 mg/kg group, 163+/−40 mg/dl for the 0.04 mg/kg group and 190+/−40 mg/dl for the 0.008 mg/kg group) compared to the negative control rats. The blood glucose profile during the OGTT at day 21 for 5F12G1 was similar to the profile at day 0 (see FIG. 32B) with blood glucose levels peaking at 312 mg/dL for 0.2 mg/kg, 355 mg/dL for 0.04 mg/kg and 400 mg/dL for 0.008 mg/dL at 30 minutes, then decreasing. Rats treated with exenatide had OGTT profiles similar to the low dose of 5F12G1. These results suggested that treatment with the anti-BKB2R mAb 5F12G1 prevented or delayed insulin resistance and the onset of type 2 diabetes.

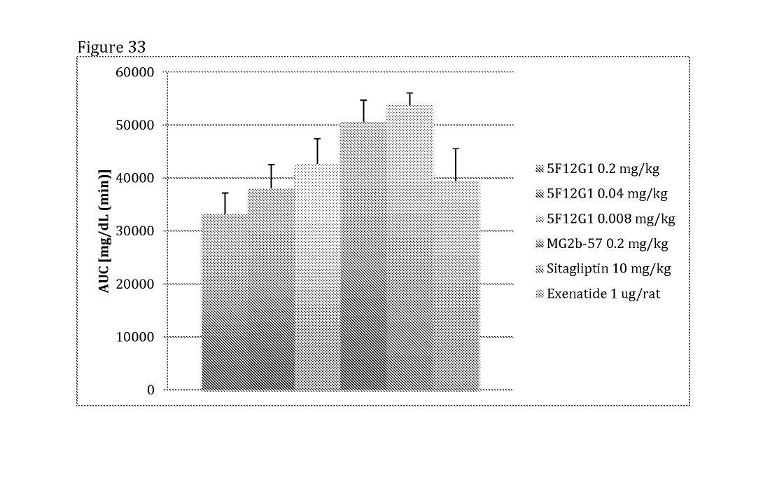

The total blood glucose levels measured during the above-described OGTT were expressed as the area under curve (AUC). Rats in all the groups at day 0 had a range of AUC blood glucose of 27044-31167 (mg/dL (min)) see FIG. 33. On days 7, 14 and 21, as expected, the blood glucose levels in the negative control group (MG2b-57) increased due to the development of type 2 diabetes, resulting in significantly higher glucose AUC in each subsequent OGTT (data not shown). By day 21, rats treated with MG2b-57 had AUC amounts of 50569.88+/−4124.62 mg/dL (min), the sitagliptin treatment group had AUC amounts of 53765.75+/−2281.45 mg/dL (min), which was equal to the blood sugar AUC obtained with exenatide (39450.13+/−6087.89 mg/dL (min)). In contrast, the 5F12G1 (0.2 mg/kg) treatment group had an AUC glucose of 33241.13+/−3910.62 mg/dL (min) on day 21, and statistically lower blood sugar AUC on days −7, 14 and 21 compared to sitagliptin and MG2b-57. Rats treated with 5F12G1 had AUC blood glucose levels on days −7, 14 and 21 that were similar to day 0, indicating treatment with 5F12G1 prevented the further development of insulin resistance and maintenance of glucose control.

Insulin levels in the ZDF rats were expected to decrease significantly past 11 weeks of age. The mean plasma insulin concentrations measured during the OGTT on day 0 and are presented in FIG. 34A. As expected, no significant differences were observed on day-0 between the groups, and mean fasting insulin levels were approximately 8-11 ng/ml, which during the OGTT increased to approximately 15-19 ng/ml at 15 minutes. However, at day −7, rats treated with the negative control MG2b-57 had significantly decreased insulin levels compared to day 0 during fasting and during the OGTT. Animals treated with 5F12G1 had insulin levels during the OGTT on day 7 comparable to day 0. By day −21, animals treated with 5F12G1 at 0.2, 0.04 and 0.008 mg/kg had insulin levels that were comparable to day 0, and significantly higher insulin levels as compared to MG2b-57, sitagliptin and exenatide (see FIG. 34B). Animals treated with 5F12G1 at 0.2, 0.04 and 0.008 mg/kg had fasting insulin levels of 17+/−5, 12+/−3 and 14+/−3 ng/ml, respectively, that increased to 30+/−7, 26+/−3 and 24+/−6 ng/ml at 15 minutes of the OGTT and returned to baseline. In contrast, animals in the negative control group had fasting insulin levels of 4+/−1.6 ng/ml that increased to 9+/−2.8 at 15 minutes. Rats treated with sitagliptine and exenatide had fasting insulin levels of 10+/−3.5 and 7+/−2.5 ng/ml respectively, that increased to 15+/−4 ng/ml in both groups at 15 minutes and slowly decreased. The detection of near normal levels of insulin secretion in groups treated with 5F12G1 at day 21 was likely due to maintenance of insulin sensitivity (prevention of insulin resistance, hyperinsulinemea), glycemic control and overall beta cell function.

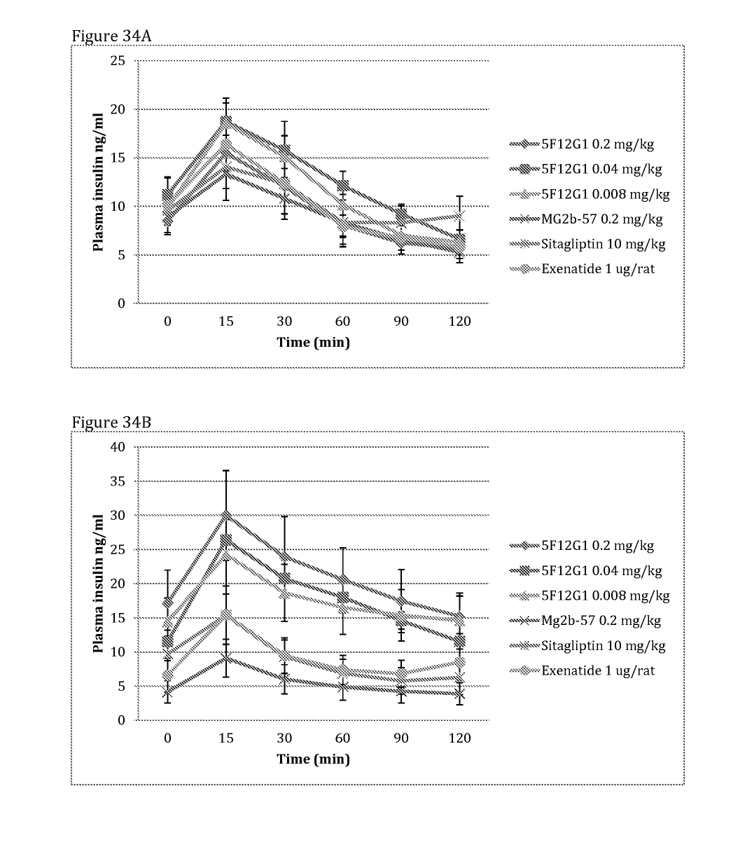

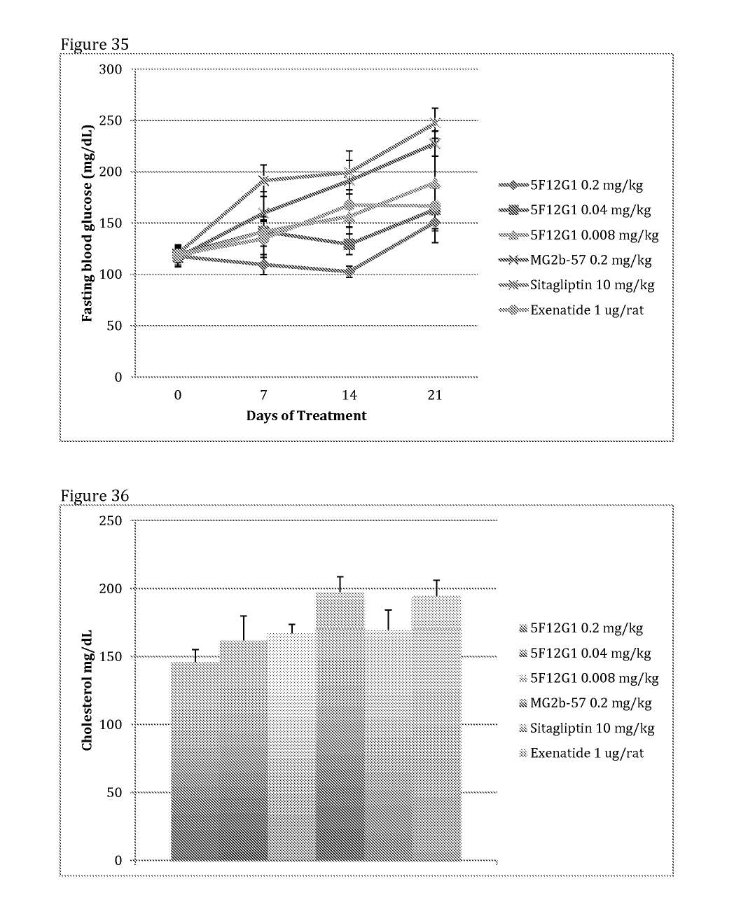

The ZDF fa/fa rats were expected to have slightly elevated fasting blood glucose level at the start of the study. This elevation in fasting blood glucose level was expected to increase with the age of the rat. Fasting blood glucose levels were measured on Day 0, 7, 14 and 21. Fasting blood glucose levels in all groups were approximately 117-120 mg/dl at day 0. As expected, fasting blood glucose levels in the negative control group increased at day 7, 14, and 21, as did the levels in the sitagliptin group. By day 21, the fasting blood glucose level in the negative control (MG2b-57) group and sitagliptin groups increased from a baseline of 116.5+/−25.8 mg/dl to 227.5+/−34.3 mg/dl and 247+/−14 mg/dl, respectively (see FIG. 35), an increase of 111.0+/−12.1 mg/dl for MG2b-57. The fasting blood glucose levels in 5F12G1 group (0.2 mg/dl) only increased from 117.6+/−14.2 mg/dl to approximately 150.8+/−56.5, 163+/−21 and 190+/−40 mg/dl by day 21, respectively, an increase of 33.1+/−19.7 mg/dl from baseline. The ZDF rats treated with high doses of 5F12G1 had a significantly lower increase in fasting blood glucose levels (p=0.0058) compared to negative control animals. Fasting blood glucose levels for the exenatide-treated group also increased a relatively small amount, to 167+/−22 mg/dl at day 21. Treatment with 5F12G1 protected against an increase in fasting blood glucose levels in a dose dependent manner. The protection by 5F12G1 from development of type 2 diabetes, as measured by fasting blood glucose levels, was similar to exenatide and improved over sitagliptin, and was indicative of maintenance of glycemic control and insulin sensitivity.

Body weights were measured prior to dosing and twice a week thereafter using a laboratory balance. The ZDF rats at 11 weeks of age had not reached their maximum body weight and were expected to increase in weight. Animals treated with 5F12G1 at all dosage groups had an approximate 13+/−1 percent increase in body weight by day 21, where as animals in the negative control, exenatide and sitagliptin treated groups had a 10+/−2 percent increase in body weights at day 21. The body weight increase in animals treated with 5F12G1 was likely due to improved health of the animals, specifically prevention of type 2 diabetes development.

Food and water intakes were measured twice a week by providing measured amounts of food and water and subtracting the measured amounts of leftover food and water. Food consumption was slightly lower in the 5F12G1 groups (all dosage groups) and differed significantly as compared to MG2b-57, sitagliptin and exenatide treated groups. All animals had food consumption of approximately 29-30 g/rat/day on day 0. By day 21, food consumption was slightly higher with MG2b-57 33+/−1 g/rat/day, exenatide 31+/−1 g/rat/day and sitagliptin treated groups 31+/−2 g/rat/day compared to the 5F12G1 groups (28+/−1 g/rat/day at 0.2 mg/kg, 27+/−2 g/rat/day at 0.04 mg/kg and 30+/−0.5 g/rat/day at 0.008 mg/kg). However, water consumption was significantly increased in animals treated with MG2b-57 (59+/−10 ml/rat/day), exenatide (48+/−4 ml/rat/day) and sitagliptin (48+/−7 ml/rat/day) treated groups, compared to animals treated with 5F12G1 in all three dosage groups (26+/−3 ml/rat/day at 0.2 mg/kg, 40+/−10 ml/rat/day at 0.04 mg/kg and 28+/−4 ml/rat/day). The increased water consumption in the negative control and sitagliptin group may have been due to higher blood glucose levels, which would result in polyuria. Decreased water consumption in the 5F12G1 treatment group may have indicated better glycemic control, and that the animals had not developed diabetes. The decreased food consumption and increased weights of animals treated with 5F12G1 compared to control animals may also indicate better glycemic control.

Serum Collection. On Day 0, 7, 14 and 21, blood samples were collected from the fasted rats in serum separator tubes (BD Biosciences, USA) by tail nip, and the blood allowed to stand at room temperature for 30 minutes. The samples were then centrifuged and the serum supernatant were transferred into 0.5 ml Eppendorf™ microfuge tubes by pipette and stored at −80° C. for the analysis of total cholesterol and triglyceride levels.

Plasma Collection. On days 0, 7, 14 and 21 during an OGTT test, blood samples were collected from the rats at each time point (0, 15, 30, 60, 90 and 120 minutes) into tubes containing lithium heparin (BD Biosciences, USA) by tail nip and kept on ice. The samples were then centrifuged at 4° C. for plasma separation and the plasma supernatants were transferred into 0.5 ml Eppendorf™ tubes by pipette and stored at −80° C. for the analysis of insulin levels.

Urine collection. On days 7 and 14 (24 hours post OGTT) urine samples were collected from each rat by spot collection method. Urine samples were analyzed for glycosuria using a glucose auto kit (Wako Chemicals USA, Inc.) according to the manufacturer's instruction.

Analysis of Plasma, Serum and Urine. As mentioned above, left untreated, the ZDF rats eventually exhibited hyperlipidemia, hypertriglyceridemia and hypercholesterolemia resulting in mild hypertension. Serum samples were analyzed for triglyceride and total cholesterol concentrations using Wako kits (Wako Chemicals USA, Inc. Richmond, Va.). Urine samples were analyzed using a Glucose Auto kit (Wako Chemicals USA, Inc. Richmond, Va.). Total cholesterol levels were measured in serum on days 0, 7, 14 and 21 (see FIG. 36). Total cholesterol on day 0 at 11 weeks of age ranged from 144-169 mg/di in the ZDF rats, or approximately 2 fold higher than in normal rats. As expected, animals treated with MG2b-57 had significantly higher serum cholesterol levels (198+/−11 ml/dl) at day 21, an increase of 28+/−11 mg/dl from baseline, which were similar to serum cholesterol levels measured in exenatide treated rats at day 21 (195+/−11 ml/dl). Serum cholesterol in animals treated with 0.2 mg/kg 5F12G1 decreased during treatment and was 145+/−26 mg/dl on day 21, a decrease of 12+/−8 mg/dl from baseline. Serum cholesterol in the 0.04 and 0.008 mg/kg 5F12G1 treatment groups increased slightly through the study, and by day 21 were 162+/−18 and 167+/−7 mg/dl, respectively. Serum cholesterol in the sitagliptin treatment groups 170+/−14 mg/dl by day 21. Treatment with 5F12G1 prevented the development of hypercholesterolemia in ZDF rats compared to negative controls, and in the highest dosage group of 5F12G1 the difference was statistically significant (p=0.0156).

Triglyceride levels were measured on days 0, 7, 14 and day 21. Serum triglyceride levels on day 0 were between 600 and 750 mg/dl, or approximately three-fold higher in the ZDF rats compared to normal rats. No significant differences were observed in serum triglyceride levels between any of the groups throughout the study.

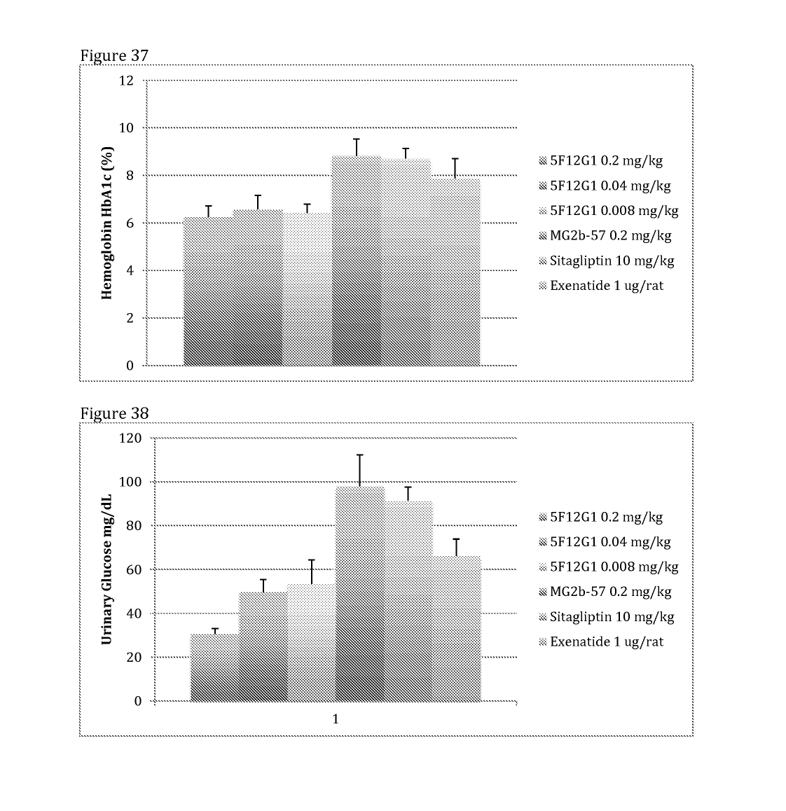

The percent of glycosylated or glycated hemoglobin A1c (HbA1c) was measured on day 21, and the mean values are presented in FIG. 37. HbA1c levels in ZDF fa/fa rats were expected to increase as the animal become hyperglycemic with age. As expected, by day 21, significantly higher percentages of HbA1c were detected in animals treated with MG2b-57 (8.8+/−0.7%), and similar percentages of HbA1c were detected in the exenatide (7.9+/−0.8%) and sitagliptin (8.8+/−0.4%) treatment groups Significantly lower percent HbA1c was detected in all dosage groups of 5F12G1, with the percentage at 6.3+/−0.5% for the high dose 5F12G1 group, and slightly higher amounts for lower dosage groups. The difference in percent HbA1c between the high dose of 5F12G1 and negative control animals was −2.58+/−0.85, and was statistically significant (p=0.0103). These results were consistent with lower blood glucose levels being detected in rats treated with 5F12G1, and suggested that 5F12G1 offers better protection against increased HbA1c in the ZDF fa/fa rats than either exenatide or sitagliptin.

The ZDF fa/fa rats were expected to have increased urine glucose levels as the study progressed. As rats develop type 2 diabetes, increased blood glucose levels eventually result in appearance of excess glucose in the urine. Urinary glucose levels were measured on day 7 and 14, and the day 14 results are presented in FIG. 38. On day 14, significant differences were observed, with the highest levels of glucose detected in urine in rats treated with MG2b-57 (98+/−14 mg/dL), and elevated urine glucose was also detected in rats treated with exenatide and sitagliptin. All groups treated with 5F12G1 at 0.2, 0.04 and 0.008 mg/kg (31+/−2, 49+/−6, and 54+/−11 mg/dL respectively) had significantly lower urine glucose levels compared to MG2b-57, exenatide and sitagliptin. These results further confirmed that treatment with 5F12G1 prevented the development of hyperglycemia in the rats.

Blood pressure measurements were performed using a blood pressure monitor and data acquisition software. The measurements were performed on days 0, 7, 14 and 21 by placing the rat in a specialized restrainer for approximately 10 to 15 minutes prior to blood pressure monitoring, with a warming pad to control the temperature. The occlusion cuff was then slid on to the base of the tail, followed by the VPR (Volume Pressure Recording) sensor cuff. The VPR sensor utilized a differential pressure transducer to non-invasively measure the blood volume in the tail, and determined systolic blood pressure, diastolic blood pressure, and heart rate. Five readings were taken per rat and the data were presented as an average.

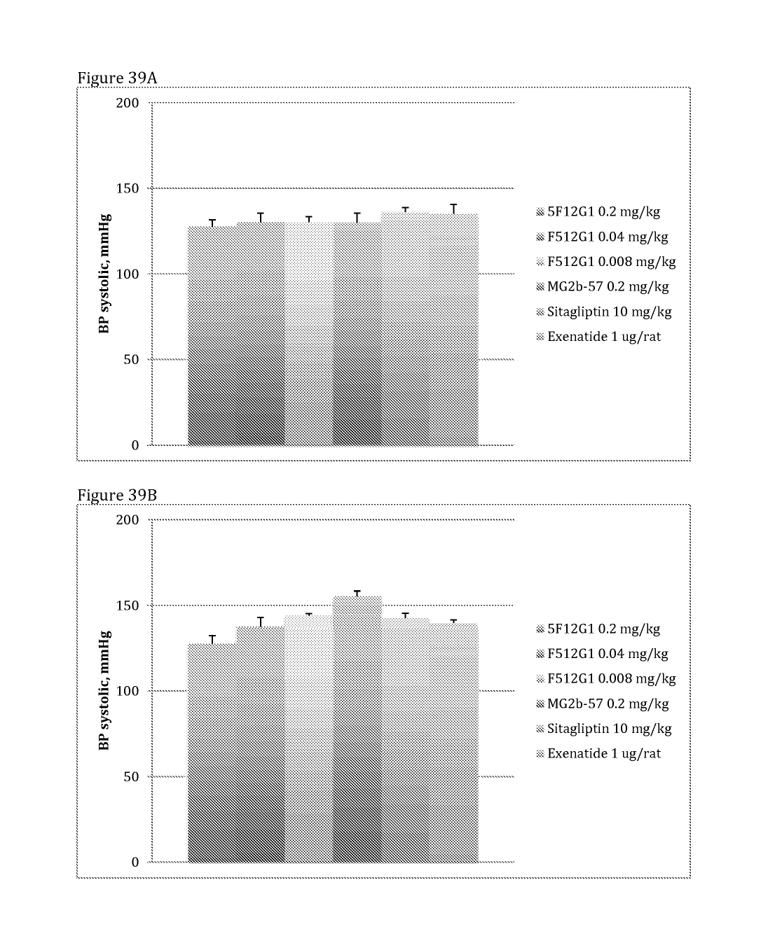

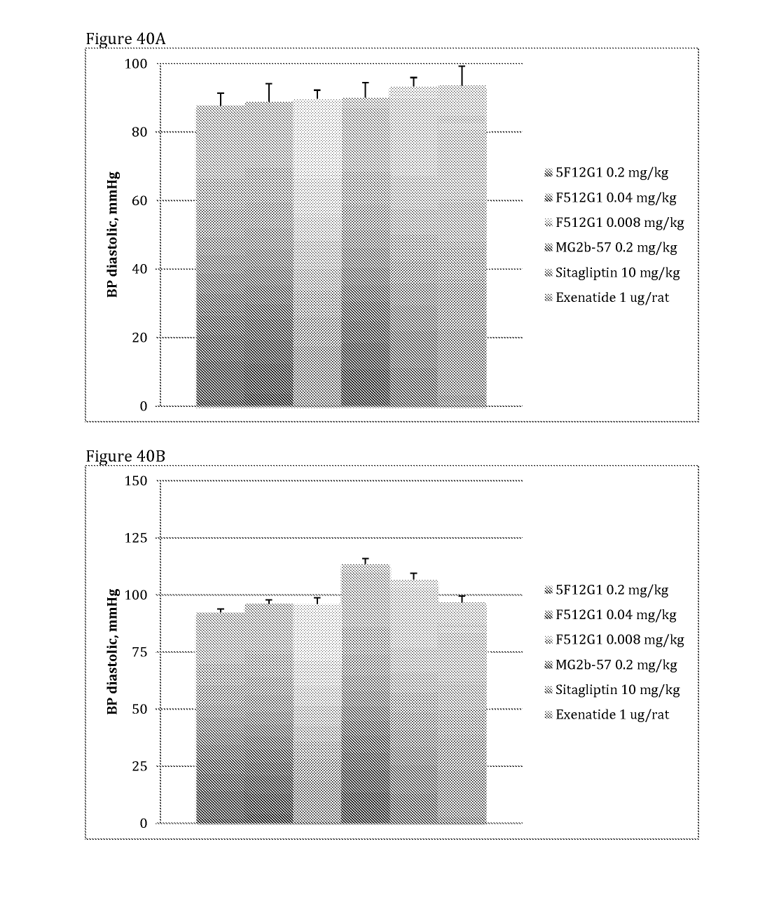

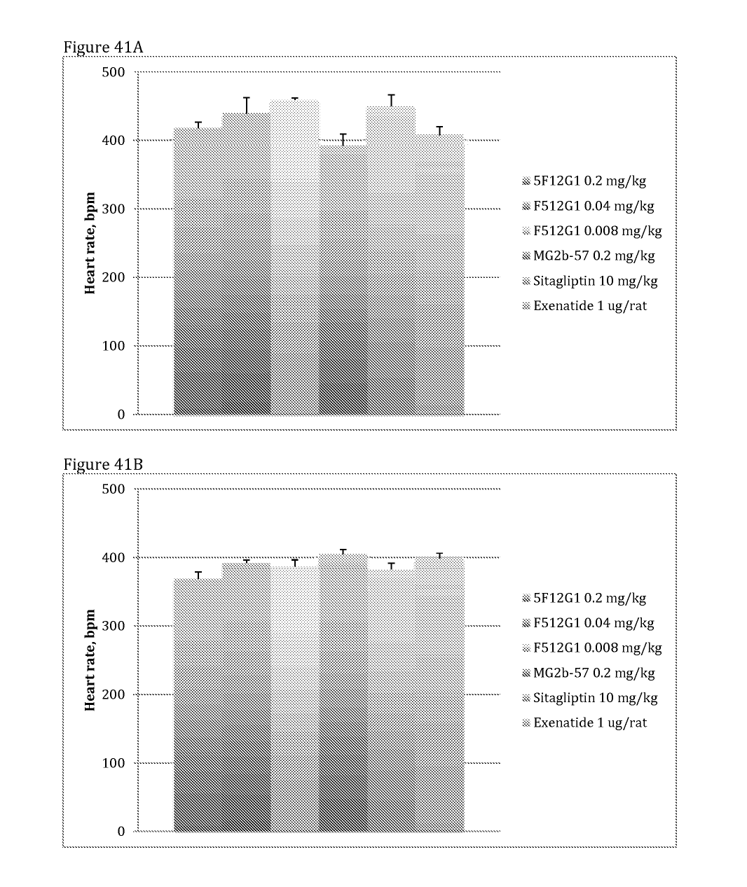

Systolic, diastolic blood pressure and heart rate were monitored on days 0, 7, 14 and 21, and the data are presented in FIGS. 39, 40 and 41. As expected, on Day-0, no significant differences were observed among the groups in measurements of systolic, diastolic blood pressure and heart rate (FIGS. 39A, 40A and 41A). All animals receiving 5F12G1 doses were observed on day 21 (see FIGS. 39B, 40B and 41B) to have systolic, diastolic blood pressure and heart rate measurements that were below the control group, MG2b-57. Treatment with 5F12G1 resulted in lower systolic, and diastolic blood pressure and also lower heart rate in the ZDF fa/fa rats, likely through the prevention of the onset of Type 2 diabetes. Specifically, treatment with 5F12G1 at the highest dose resulted in an increase in systolic blood pressure of 0.12+/−4.4 mm Hg from baseline, compared to the negative control group which had an increase of 25.5+/−2.9, the difference being statistically significant (p=0.0004).

Hyperinsulinemic-Euglycemic Clamp Study. The gold standard for investigating and quantifying insulin resistance is the hyperinsulinemic-euglycemic clamp, so-called because it measures the amount of glucose necessary to compensate for an increased insulin level without causing hypoglycemia. After 21 days of treatment, animals in the sub-groups (N=4 per treatment) that were not subjected to prior testing were fasted overnight and a 120-minute hyperinsulinemic-euglycemic clamp study was performed on animals in the sub-groups. Animals were anesthetized and maintained throughout the procedure under isoflurane anesthesia. The saphenous vein and femoral artery were catheterized. The saphenous vein catheter was used to infuse human insulin (Humalin® R, Eli Lilly, Indianapolis, Ind.) and a 20% glucose solution. The femoral artery catheter was used to collect blood samples and monitoring of arterial blood glucose levels. At the start of the clamp study, insulin was infused at a constant rate of 8 mU/kg/minute. In order to compensate for the resulting drop in blood glucose levels from the insulin infusion, a 20% glucose solution was infused at variable rates, adjusted every 10 minutes, to maintain a target blood glucose level. Arterial blood glucose levels were measured prior to infusion at t=−120, −90, −30, and 0 minutes and then at every 10 minutes for 120 minutes (t=120), using a glucose meter (Accu-Chek®, Roche Diagnostics). The glucose infusion rate (GIR) during the test determined insulin sensitivity. If a high GIR was required to compensate for the insulin infusion, then the animal was considered insulin-sensitive. If a low GIR was required, the animal was considered resistant to insulin action.

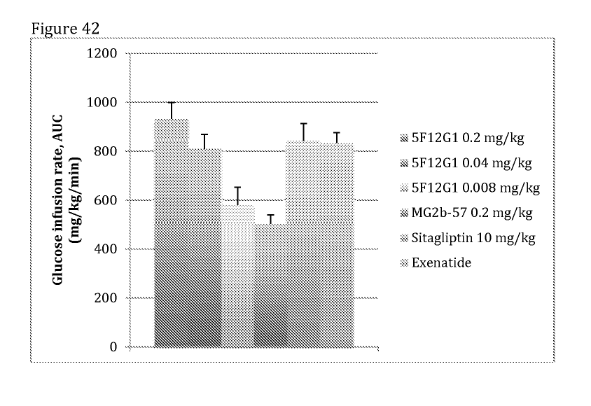

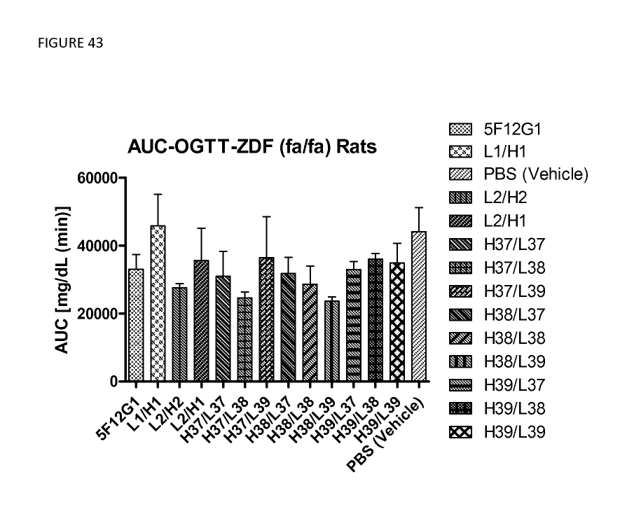

The glucose infusion rate (GIR), AUC-GIR and arterial blood glucose were measured during the hyperinsulinmic-euglycemic clamp study and data for the AUC-GIR are presented in FIG. 42. As expected, rats treated with the negative control, MG2b-57, were resistant to insulin and had a low AUC-GIR. Animals treated with 5F12G1 at 0.2 and 0.04 mg/kg had significantly higher AUC-GIR, indicating the treatment had preserved insulin sensitivity in these animals after 21 days. The AUC-GIR for groups treated with sitagliptin and exenatide were similar to the 5F12G1 high dose treatment group. Treatment with 5F12G1 for 21 days preserved insulin sensitivity in the ZDF fa/fa rats.

Data Analysis. Data are presented as the mean±standard error (SEM) obtained from Microsoft Excel or Graph Pad Prism version 5.00 for Windows (Graph Pad Software, San Diego Calif. USA). P values were calculated using T Test analysis on Graph Pad Prism® software. Differences between groups were considered significant at P<0.05.

The mean change in fasting blood glucose (mg/dL), serum cholesterol (mg/dL) and systolic blood pressure (mm Hg) from baseline (Day 0) to Day 21 was compared between the high-dose (5F12G1, 0.2 mg/kg) and negative control (MG2b-57, 0.2 mg/kg) groups using an analysis of covariance to adjust for baseline levels. The mean HbA1c percentage in high-dose (5F12G1, 0.2 mg/kg) rats was compared to the mean HbA1c percentage for negative control (MG2b-57, 0.2 mg/kg) rats using an unequal-variance, independent two-sample t-test. A significance level of a=0.05 was used for all tests, and all analyses were conducted using SAS statistical software (vs. 9.2, Cary, N.C., U.S.A.).

Overall, administration of 5F12G1 at different doses was well tolerated and no toxic effects were noted.

Administration of 5F12G1 at 0.2 and 0.04 mg/kg daily for 21 days to ZDF fa/fa rats prevented the development of insulin resistance, and maintained glycemic control as measured by OGTT, insulin secretion, blood glucose levels and HbA1c. Animals treated with MG2b-57, sitagliptin and exenatide all had significant deterioration in the above parameters. 5F12G1 treatment also prevented increases in blood pressure, heart rate, triglyceride and cholesterol levels as compared to the other treatment groups.

### Example 11

### Sequence of Anti-BKB2R Antibody

This example describes sequencing of the murine monoclonal anti-BKB2R antibody, 5F12G1. Total RNA from hybridoma 5F12G1 was extracted using an RNAeasy™ kit according to the manufacturer's instructions (Qiagen, Valencia, Calif.). cDNA was synthesized by a modification to the method described in the instructions for 5′-RACE™ kits (SMART RACE cDNA kit, Clontech, Mountain View, Calif.), using MMLV reverse transcriptase.

5′-RACE PCR was performed as described (Clontech SMART RACE™ kit) using one of the following as the RACE-specific primer: MOCG12FOR(CTC AAT TTT CTT GTC CAC CTT GGT GC) (SEQ ID NO:61) for Mouse IgG1, IgG2a, MOCG2bFOR(CTC AAG TTT TTT GTC CAC CGT GGT GC) (SEQ ID NO:62) for Mouse IgG2b, MOCG3FOR(CTC GAT TCT CTT GAT CAA CTC AGT CT) (SEQ ID NO:63) for Mouse IgG3 MOCMFOR (TGG AAT GGG CAC ATG CAG ATC TCT) (SEQ ID NO:64) for IgM, CKMOsp (CTC ATT CCT GTT GAA GCT CTT GAC AAT GGG) (SEQ ID NO:65) for Mouse kappa, CL1 FORsp (ACA CTC AGC ACG GGA CAA ACT CTT CTC CAC AGT) (SEQ ID NO:66) for Mouse Lambda 1, CL2FORsp (ACA CTC TGC AGG AGA CAG ACT CTT TTC CAC AGT) (SEQ ID NO:67), and CL4FORsp (ACA CTC AGC ACG GGA CAA ACT CTT CTC CAC ATG) (SEQ ID NO:68). (A Bradbury, Cloning Hybridoma cDNA by RACE, Antibody Engineering 2^{nd} Edition 2010).

cDNA was sequenced from both ends using standard chain-termination technology as well as cloned into pCR-Topo2.1 using the Topo TA cloning kit (Life Technologies). Clones containing the cDNA were sequenced using M13rev (TCACACAGGAAACAGCTATGA) (SEQ ID NO:69) and T7-forward primers (TAATACGACTCACTATAGG) (SEQ ID NO:70).

The resulting sequences were the murine 5F12G1 immunoglobulin heavy chain variable region domain encoding sequence set forth in SEQ ID NO:49, and the murine 5F12G1 immunoglobulin light chain variable region domain encoding sequence set forth in SEQ ID NO:50. The deduced translated amino acid sequence for the murine 5F12G1 immunoglobulin heavy chain variable region domain is set forth in SEQ ID NO:1, and the deduced translated amino acid sequence for the murine 5F12G1 immunoglobulin light chain variable region domain is set forth in SEQ ID NO:2.

The murine hybridoma mAb, 5F12G1, which specifically bound to the human BKB2R and exerted an agonist effect, as disclosed herein, was then humanized to obtain an anti-BKB2R monoclonal antibody that would avoid potential human immune reactions (immunogenicity) against the mouse monoclonal antibody, to allow for multiple injections and/or long-term use of the antibody in humans.

The antibody humanization process was accomplished by inserting the appropriate mouse complementarity determining region (CDR) coding segments, responsible for the desired binding properties, into a human antibody “scaffold”. The three mouse CDR regions in the heavy chain (SEQ ID NOS:43-45) and three CDR regions in the light chain (SEQ ID NOS:46-48) of the antibody were identified using the Kabat method (Kabat E A, et al. (1991)) Sequences of Proteins of Immunological Interest, Fifth Edition. NIH Publication No. 91-3242) and grafted into the VH and VL human donor scaffold regions. The CDR grafting approach was first described for humanization of a mouse antibody (Queen, et al. Proc Natl Acad Sci USA. (1989) December; 86(24):10029-33) and was recently reviewed by Tsurushita and Vasquez (2004) and Almagro and Fransson (2008) (Tsurushita N, et al., J Immunol Methods. 2004 December; 295(1-2):9-19; Almagro J C, and Fransson J. Front Biosci. (2008) 13:1619-33).

To determine the human antibody gene sequence that could best accept the mouse CDRs and still allow binding to the epitope, the surrounding Fv regions in the mouse 5F12G1 monoclonal antibody sequence were analyzed, and a best-fit method was used to select the most appropriate donor human gene sequence using proprietary methodology provided by Panorama Research Inc. (Sunnyvale, Calif., USA) and LakePharma, Inc. (Belmont, Calif., USA).

Briefly, human antibody framework sequences were used that were germline or close to germline. The human VH sequences that were related to germline genes VH3-33, VH3-73, VH3-7, among others, provided the best matches. The human VL sequences that were related to germline genes VK2-28, VK2-30, among others, provided the best matches. Several 3D models of the Fv of the target antibody were built using combinations of light chain and heavy chain variable domains to produce models. Some of the considerations that were used to choose the backbone were that the human templates matched the CDR lengths and canonical structures with those predicted from the mouse 5F12G1 sequence. Amino acid positions were identified in the framework regions that differed between murine and human and that may have influenced antigen binding. That certain antibody genes exhibited high usage of the framework backbones in the human antibody repertoire was a positive factor for selection, as was good conservation at structurally significant framework positions relative to other germline choices.

Proprietary humanization optimizations performed by Panorama Research Inc. (Sunnyvale, Calif., USA) yielded the humanized anti-BKB2R immunoglobulin heavy (H1, H2), and light (L1, L2) chain variable region domains set forth in the Sequence Listing as SEQ ID NOS:3-4 and 8-9, respectively. Proprietary humanization optimizations performed by LakePharma, Inc. (Belmont, Calif., USA) yielded the humanized anti-BKB2R immunoglobulin heavy (H37, H38, H39), and light (L37, L38, L39) chain variable region domains set forth in the Sequence Listing as SEQ ID NOS:5-7 and 10-12, respectively.

Five different versions of humanized light chains and five versions of humanized heavy chains were thus created from both instances above, based on the mouse 5F12G2 clone, and the amino acid and encoding polynucleotide sequences, including CDRs, V regions, and H and L chains, are set forth in the Sequence Listing as SEQ ID NOS:3-48, 51-60, and 75-92.

### Example 12

### Expression and Purification of Humanized Anti-Human BKB2R Monoclonal Antibodies

H1, H2, L1, L2

Coding sequences, respectively, SEQ ID NO:51, 52, 56 and 57, for the H1 (SEQ ID NO:3), H2 (SEQ ID NO:4), L1 (SEQ ID NO:8) and L2 (SEQ ID NO:9) humanized variable region sequences, were synthetically made into DNA constructs (BioBasic, Markham Ontario). The DNA sequences for the H1 and H2 heavy chains were each cloned into a pDH2 vector in frame with a human IgG2 Fc region. Similarly, the L1 and L2 humanized light chains were each cloned into a pDH2 vector in frame with the human kappa constant region. Various combinations of humanized VL, VH or chimeric VL and VH (mix and match approach) were transiently transfected into at least 100 mls of 293-derived cells (e.g., 293F) using standard lipid-based transfection protocol. Specifically, the vector encoding the sequence H1 was co-transfected with the vector encoding L1, or the vector encoding L2, or the original mouse 5F12G1 VL. Similarly, the vector encoding H2 was co-transfected with vectors encoding L1 or L2, and the vector encoding original mouse 5F12G1 VH was co-transfected with L1, or L2. HEK-293 cells were cultivated in suspension culture using Gibco's Freestyle serum-free medium. The cultures were incubated at 37° C. in an atmosphere comprising 5% CO₂ and 95% air. The 100 mL test expressions were produced using 500 mL sterile, disposable Corning Erlenmeyer flasks and the 500 mL and 1-liter expressions were conducted using 3-liter Corning sterile disposable flasks. The suspension cultures were placed on a platform shaker with an agitation rate of 100 rpm. When the cell density reached 1.5×10⁶ cells per mL the cultures were transfected with the selected plasmid pair. Polyethyleneimine (PEI, 25 kDa linear, Polyplus Transfections) was used as the transfection reagent in a ratio of 4:1 with plasmid DNA. A total of 1 mg plasmid was used for each liter of culture. The transfected cells were incubated for 120 hours and the supernatant was harvested and sterile filtered using 0.2 micron vacuum filter units (Nalgene). The sterile supernatant was stored at 2-8° C. prior to purification.

The recombinant IgG present in the culture supernatant was purified using affinity chromatography. For each 100 mL expression, 1 mL of Protein G Sepharose Fast Flow (GE Bioscience) was equilibrated using PBS pH 7.4 and added directly to the supernatant. The IgG was batch absorbed at 2-8° C. for 16 hours with gentle agitation. After incubation the resin/supernatant mixture was transferred to a conical centrifuge bottle and the resin was allowed to settle. The supernatant (flow-through) was decanted and the resin was transferred to a disposable column (GE). The resin was washed with 20 volumes of PBS using gravity flow. The IgG was eluted in three to five fractions of 1 mL each using 0.1 M Glycine pH 3.0. A volume of 1M Tris pH 9.0 was added to each fraction tube to neutralize the pH of the glycine buffer. The eluate samples and the flow-through were analyzed by SDS PAGE (Coomassie stain) and fractions containing the IgG were pooled. The pooled eluates were diafiltered and concentrated into PBS using centrifugal ultrafilters (Millipore Centricon, 50 kDa MWCO). If possible the final products were pfilter sterilized using 0.2 micron syringe filter units (Millipore PES). The protein concentration of each sample was determined using A₂₈₀ absorbance and an extinction coefficient of 1.4. The samples were stored at 2-8° C. prior to shipment. The conditions used for the 500 mL and 1-liter cultures were identical to those outlined above with the exception that 4 mL of resin was used to capture the IgG. Plasmid pairs were expressed as summarized in Table 7.

TABLE 7
Humanized H + L Chains

| | Purified | Scale | |
|---|---|---|---|
| Plasmid Pairs | rIgG, mg/L | (L) | mg IgG |
| L1/H1 | 0.56 | 0.1 | 0.056 |
| L1/HC | 0.48 | 0.1 | 0.048 |
| L2/HC | 0.53 | 0.1 | 0.053 |
| L2/H1 | 1.28 | 0.1 | 0.128 |
| L1/H2 | 1.13 | 0.1 | 0.113 |
| L2/H2 | 1.61 | 0.1 | 0.161 |
| L1/H1 | 4.00 | 2 | 8.2 |
| L2/H2 | 4.00 | 1 | 4.2 |
| L2/H1 | 0.75 | 0.5 | 038 |

A non-reduced SDS-PAGE gel of the various purified IgG preparations yielded an electrophoretogram demonstrating the expected weight of an intact IgG antibody, thus confirming proper antibody expression and purification.

H37, H38, H39, L37, L38, L39

A similar strategy to that described above was used to generate combinations of humanized heavy chain H37 with L37, L38, L39; H38 with L37, L38 or L39, and H39 with L37, L38 or L39. The VH and VL sequences were cloned in frame into pcDNA 3.3 vectors encoding a human IgG2 heavy or light chain constant region. The plasmids containing the full-length heavy chain and light chain sequences were transfected into CHO cells with Lafectine transfection reagent (LakePharma catalog number 4502030). Supernatants were collected four days after transfection, and the total IgG levels in supernatants were determined using Fc ELISA (LakePharma catalog number 2001002). Supernatants from CHO transient transfections were purified using a protein A ligand on the MabSelect SuRe™ beads (GE Healthcare). Antibodies captured by beads were eluted by acetic acid pH 3.0, and stored in 200 mM Tris pH 7.5, 0.4% sodium acetate and 150 mM NaCl. Antibody preparations contained isolated proteins (approximately 0.3-0.85 mg) at concentrations of approximately 0.9 to 1.7 mg/ML, and SDS-PAGE analysis demonstrated purities of greater than 95%, with the expected heavy (50 kDa) and light chain (25 kDa) molecular weights.

### Example 13

### Binding Affinity and Avidity

Proteins corresponding to all combinations of humanized or chimeric (5F12G1 VH and VL on human IgG2 backbone) antibodies were tested for binding to the human BKB2R-derived epitope peptide, SE ID NO:73.

A ForteBio (Menlo Park, Calif., USA) Octet® platform was used to analyze the binding affinities and binding characteristics of the humanized monoclonal antibodies to the peptide epitope (SEQ ID NO:73) and compared to the original mouse monoclonal 5F12G1. This platform employed label-free technology for measuring biomolecular interactions by optical analysis of the interference pattern of white light reflected from two surfaces: a layer of immobilized protein on the biosensor tip, and an internal reference layer. Any change in the number of molecules bound to the biosensor tip caused a shift in the interference pattern that was measured in real-time. Binding specificity, and rates of association and dissociation were monitored.

For the Octet study, antibodies were analyzed by kinetic titration of the antibodies. Antibodies were prepared in kinetic buffer (0.001 M phosphate buffered saline (NaCl 0.0138 M; KCl-0.00027 M); pH 7.4, at 25° C., 0.1 mg/ml BSA, 0.002% Tween and 0.005% Sodium Azide) followed by 1:2 serial dilutions.

Sensor Prep: Streptavidin biosensors (FortéBio Inc, Menlo Park, Calif.) were coated by incubation in a solution containing a peptide (Seq ID No. 2-PEG-biotin) (Biosyn, Lewisville, Tex.) at 50 μg/ml (300 seconds/1000 rpm shaking). 96 well half-volume plates were used for testing. 90 μl of sample was plated per well. Sensors were allowed to equilibrate to baseline in kinetic buffer (60 seconds/1000 rpm). The sensors were then placed into the various antibody dilutions to allow binding (association) to the probe for 500 seconds/1000 rpm, and measurements were taken. The sensors were then moved into kinetic buffer without antibody for dissociation (500 seconds/1000 rpm), and measurements were taken. Octet system software calculated kinetic constants for on rate/off rate/affinity.

The control mouse antibody 5F12G1 (sample No. ab 404) was tested at an initial concentration of 3000 nM (450 μg/ml) followed by 1:2 dilutions. For test humanized monoclonal antibodies, the highest concentration used was 500 nM followed by 1:2 dilutions. Each concentration was tested twice. The HC and LC represented the mouse original 5F12G1 VH and VL sequences. Exemplary data are presented in Tables 8 and 9.

TABLE 8
Antibody binding data

| Sample | Conc. | Max | | | |
|---|---|---|---|---|---|
| ID | (nM) | Response | KD (M) | kon(1/Ms) | kdis(1/s) |
| ab 404 | 3333 | 0.0757 | 4.62E−07 | 2.35E+03 | 1.09E−03 |
| ab 404 | 1667 | 0.0544 | 4.62E−07 | 2.35E+03 | 1.09E−03 |
| ab 404 | 1000 | 0.0385 | 4.62E−07 | 2.35E+03 | 1.09E−03 |
| ab 404 | 833 | 0.0265 | 4.62E−07 | 2.35E+03 | 1.09E−03 |
| L1/H1 | 500 | 0.216 | 2.33E−06 | 4.04E+05 | 9.42E−01 |
| L1/HC | 500 | 0.194 | 3.98E−06 | 5.43E+05 | 2.16E+00 |
| L2/HC | 500 | 0.1434 | 4.53E−06 | 1.45E+05 | 6.55E−01 |
| L2/H1 | 500 | 0.1715 | 4.19E−09 | 1.21E+06 | 5.07E−03 |
| L1/H2 | 500 | 0.161 | 4.03E−07 | 2.43E+06 | 9.76E−01 |
| L2/H2 | 500 | 0.171 | 2.25E−08 | 9.92E+05 | 2.23E−02 |
| L1/H1 | 250 | 0.2864 | 4.41E−07 | 1.59E+06 | 7.01E−01 |
| L1/HC | 250 | 0.1481 | 3.04E−06 | 2.39E+05 | 7.28E−01 |
| L2/HC | 250 | 0.2042 | 3.37E−07 | 2.17E+06 | 7.32E−01 |
| L2/H1 | 250 | 0.1799 | 2.03E−08 | 2.16E+05 | 4.38E−03 |
| L1/H2 | 250 | 0.174 | 2.05E−06 | 3.50E+05 | 7.19E−01 |
| L2/H2 | 250 | 0.1705 | 2.04E−08 | 1.76E+06 | 3.59E−02 |

TABLE 9
Antibody binding data

| Sample | Conc. | Max | | | |
|---|---|---|---|---|---|
| ID | (nM) | Response | KD (M) | kon(1/Ms) | kdis(1/s) |
| H37/L37 | 500 | 0.1198 | 7.58E−07 | 1.55E+06 | 1.18E+00 |
| H37/L38 | 500 | 0.1726 | 2.20E−07 | 9.47E+06 | 2.08E+00 |
| H37/L39 | 500 | 0.1215 | 1.23E−06 | 1.11E+06 | 1.98E+00 |
| H38/L37 | 500 | 0.5774 | 3.11E−06 | 9.44E+06 | 2.94E+01 |
| H38/L38 | 500 | 0.1315 | 2.49E−09 | 3.06E+06 | 7.63E−03 |
| H38/L39 | 500 | 0.0681 | 9.32E−08 | 1.14E+09 | 1.06E+02 |
| H39/L37 | 500 | 0.1191 | 2.34E−08 | 1.23E+06 | 2.88E−02 |
| H39/L38 | 500 | 0.1435 | 7.61E−07 | 6.44E+06 | 4.90E+00 |
| H39/L39 | 500 | 0.0915 | 1.73E−06 | 7.29E+05 | 1.26E+00 |

Humanized monoclonal antibodies with the light chain L2 coupled with the heavy chain H1 or H2 demonstrated stronger binding (lower K_{D}) than the original mouse monoclonal antibody. Also, the combinations of H38/L38, H38/L39 and H39/L37 appeared to demonstrate stronger binding (lower KD) than the original mouse monoclonal antibody.

### Example 14

### Testing the Bioactivity of Humanized Monoclonal Antibodies

This example describes evaluation of mouse mAb 5F12G1 and its twelve humanized clones in Zucker Diabetic fatty (ZDF fa/fa) rats for effects on insulin sensitivity in animals. Zucker Diabetic Rats develop symptoms similar to type 2 diabetes and are genetically resistant to insulin. Zucker rats demonstrate excessive increases in blood glucose levels during an OGTT. Therefore, the Zucker rat is a good model to test the ability of the monoclonal antibody to increase insulin sensitivity, especially in an OGTT.

Male ZDF fa/fa rats were obtained from Charles River (Kingston, ON). Upon arrival, rats were ten weeks of age. The rats were housed individually per cage in a room with a photo cycle of 12 hours of light and 12 hours of dark and an ambient temperature of 70-72° F. and fed on regular rodent diet and water ad libitum. After seven days of acclimatization, rats were grouped into fourteen groups of three rats per group (Table 10).

TABLE 10
ZDF fa/fa Groups

| | | | | | Dosing | Dosing |
|---|---|---|---|---|---|---|
| Group | Description | N | ROA | Dose | Volume | Frequency |
| 1 | 5F12G1 | 3 | s.c | 0.2 | 200 μl | 30 min prior to |
| | Positive | | | mg/kg | | glucose |
| | Control | | | | | administration |
| 2 | L1/H1 | 3 | s.c | 0.2 | 200 μl | |
| | | | | mg/kg | | |
| 3 | L2/H2 | 3 | s.c | 0.2 | 200 μl | |
| | | | | mg/kg | | |
| 4 | L2/H1 | 3 | s.c | 0.2 | 200 μl | |
| | | | | mg/kg | | |
| 5 | H37/L37 | 3 | s.c | 0.2 | 200 μl | |
| | | | | mg/kg | | |
| 6 | H37/L38 | 3 | s.c | 0.2 | 200 μl | |
| | | | | mg/kg | | |
| 7 | H37/L39 | 3 | s.c | 0.2 | 200 μl | |
| | | | | mg/kg | | |
| 8 | H38/L37 | 3 | s.c | 0.2 | 200 μl | |
| | | | | mg/kg | | |
| 9 | H38/L38 | 3 | s.c | 0.2 | 200 μl | |
| | | | | mg/kg | | |
| 10 | H38/L39 | 3 | s.c | 0.2 | 200 μl | |
| | | | | mg/kg | | |
| 11 | H39/L37 | 3 | s.c | 0.2 | 200 μl | |
| | | | | mg/kg | | |
| 12 | H39/L38 | 3 | s.c | 0.2 | 200 μl | |
| | | | | mg/kg | | |
| 13 | H39/L39 | 3 | s.c | 0.2 | 200 μl | |
| | | | | mg/kg | | |
| 14 | PBS | 3 | s.c | xxxxx | 200 μl | |
| | (Vehicle | | | | | |
| | control) | | | | | |

Oral Glucose Tolerance Test. An oral glucose tolerance test (OGTT) was performed on overnight fasted (16 hours) rats. Rats received subcutaneously administered humanized mAbs, mouse mAb 5F12G1 (positive control) and PBS (vehicle control) at a dose of 0.2 mg/kg body weight, thirty minutes prior to glucose administration. D-Glucose was prepared in distilled water and administered orally at 2 g/kg body weight. Blood glucose levels were measured before administration of humanized mAbs, 5F12G1 or vehicle (t=−30 minutes) and just before glucose loading (0 minute), and at timepoints of 30, 60, 90 and 120 minutes, using Accu-chek™ glucose meter.

Results and Data Analysis: The data (FIG. 34) were presented as the mean±standard error (SEM) obtained from Microsoft Excel or GraphPad Prism version 5.00 for Windows (GraphPad Software, San Diego Calif. USA).

Single administration of humanized anti-BKB2R mAbs derived from 5F12G1, and of 5F12G1 (0.2 mg/kg), decreased the area under curve (AUC) of blood glucose concentration after oral administration of glucose in ZDF fa/fa rats as compared to vehicle control, except for mAb L1/H1. The decrease in AUC of blood glucose was higher with H38/L39 followed in order of effect by H37/L38, L2/H2, H38/L38, H37/L37, H38/L38, H39/137, H39/L39, H37/L37, H39/L38, H37/L39, and L1/H1. mAb 5F12G1 also showed improvement in glucose tolerance.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-34. (canceled)

35. A method for lowering blood glucose in a patient in need thereof, comprising administering to the patient an antibody, or antigen-binding fragment thereof, that specifically binds to a bradykinin B2 receptor (BKB2R) peptide epitope having the sequence set forth in SEQ ID NO:73 or 74.

36. The method of claim 35, where the patient has one or more of insulin resistance, impaired glucose tolerance (IGT), impaired insulin secretion, or excessive hepatic glucose output.

37. The method of claim 35, where the patient has diabetes.

38. The method of claim 37, where the patient has type 2 diabetes.

39. The method of claim 35, where the patient is human.

40. The method of claim 35, where the antibody, or antigen-binding fragment thereof, specifically binds to the peptide epitope of SEQ ID NO:73.

41. The method of claim 35, where the antibody, or antigen-binding fragment thereof, is selected from the group consisting of a single chain antibody, a ScFv, a univalent antibody lacking a hinge region, and a minibody.

42. The method of claim 35, where the antibody is a Fab or a Fab′ fragment.

43. The method of claim 35, where the antibody is a F(ab)₂ fragment.

44. The method of claim 35, where the antibody is a whole antibody.

45. The method of claim 35, where the antibody comprises a human IgG Fc domain.

46. The method of claim 45, where the antibody comprises a human IgG2 Fc domain.

47. The method of claim 35, where the antibody is humanized.

48. The method of claim 35, where the antibody, or antigen-binding fragment thereof, comprises a heavy chain variable region that comprises VHCDR1, VHCDR2 and VHCDR3 amino acid sequences; and a light chain variable region that comprises VLCDR1, VLCDR2 and VLCDR3 amino acid sequences, wherein at least one of:
(1) (A) VHCDR1, VHCDR2 and VHCDR3 comprise, respectively, the amino acid sequences of (i) SEQ ID NOS:19, 20 and 21, or (ii) SEQ ID NOS:22, 23 and 24; and (B) VLCDR1, VLCDR2 and VLCDR3 comprise, respectively, the amino acid sequences of (i) SEQ ID NOS:34, 35 and 36, (ii) SEQ ID NOS:37, 38 and 39, or (iii) SEQ ID NOS:40, 41 and 42; including variants thereof where at least one of said VHCDR or VLCDR amino acid sequences is modified by one amino acid substitution; or
(2) (A) VHCDR1, VHCDR2 and VHCDR3 comprise, respectively, the amino acid sequences of (i) SEQ ID NOS:13, 14 and 15, or (ii) SEQ ID NOS:16, 17 and 18; and (B) VLCDR1, VLCDR2 and VLCDR3 comprise, respectively, the amino acid sequences of (i) SEQ ID NOS:28, 29 and 30, or (ii) SEQ ID NOS:31, 32 and 33; including variants thereof where at least one of said VHCDR or VLCDR sequences is modified by one amino acid substitution.

49. The method of claim 35, where VHCDR1, VHCDR2 and VHCDR3 comprise, respectively, the amino acid sequences of (i) SEQ ID NOS:19, 20 and 21, or (ii) SEQ ID NOS:22, 23 and 24; and where VLCDR1, VLCDR2 and VLCDR3 comprise, respectively, the amino acid sequences of (i) SEQ ID NOS:34, 35 and 36, (ii) SEQ ID NOS:37, 38 and 39, or (iii) SEQ ID NOS:40, 41 and 42.

50. The method of claim 35, where VHCDR1, VHCDR2 and VHCDR3 comprise, respectively, the amino acid sequences of SEQ ID NOS:16, 17 and 18, and where VLCDR1, VLCDR2 and VLCDR3 comprise, respectively, the amino acid sequences of SEQ ID NOS:31, 32 and 33.

51. The method of claim 35, where VHCDR1, VHCDR2 and VHCDR3 comprise, respectively, the amino acid sequences of SEQ ID NOS:19, 20 and 21, and where VLCDR1, VLCDR2 and VLCDR3 comprise, respectively, the amino acid sequences of SEQ ID NOS:34, 35 and 36.

52. The method of claim 35, where VHCDR1, VHCDR2 and VHCDR3 comprise, respectively, the amino acid sequences of SEQ ID NOS:19, 20 and 21, and where VLCDR1, VLCDR2 and VLCDR3 comprise, respectively, the amino acid sequences of SEQ ID NOS:37, 38 and 39.

53. The method of claim 35, where VHCDR1, VHCDR2 and VHCDR3 comprise, respectively, the amino acid sequences of SEQ ID NOS:19, 20 and 21, and where VLCDR1, VLCDR2 and VLCDR3 comprise, respectively, the amino acid sequences of SEQ ID NOS:40, 41 and 42.

54. The method of claim 35, where VHCDR1, VHCDR2 and VHCDR3 comprise, respectively, the amino acid sequences of SEQ ID NOS:22, 23 and 24, and where VLCDR1, VLCDR2 and VLCDR3 comprise, respectively, the amino acid sequences of SEQ ID NOS:34, 35 and 36.

55. The method of claim 35, where VHCDR1, VHCDR2 and VHCDR3 comprise, respectively, the amino acid sequences of SEQ ID NOS:22, 23 and 24, and where VLCDR1, VLCDR2 and VLCDR3 comprise, respectively, the amino acid sequences of SEQ ID NOS:37, 38 and 39.

56. The method of claim 35, where VHCDR1, VHCDR2 and VHCDR3 comprise, respectively, the amino acid sequences of SEQ ID NOS:22, 23 and 24, and where VLCDR1, VLCDR2 and VLCDR3 comprise, respectively, the amino acid sequences of SEQ ID NOS:40, 41 and 42.


SEQUENCE LISTING (92 sequences)

SEQ ID NO: 1 - protein, 118 aa, Mus musculus
EVKLEESGGGLVQPGGSMKLSCVVSGFTFSDYWMSWVRHSPEMGLEWVAEIRLKSDNYATHYAESVKGRFTISRDDSKSRLYLQINSLRGEDTGIYYCTRDYYAMEYWGQGTSVTVSS

SEQ ID NO: 2 - protein, 112 aa, Mus musculus
DVVMTQTPLSLPVSLGDQASISCRSSQTIVHSNGKTYLEWYLQKPGQSPKLLIYKVSNRFSGVPDRFSGSGSGTEFTLKISRVETEDLGVYYCFQASHVPYTFGGGTKLEIK

SEQ ID NO: 3 - protein, 118 aa, Artificial Sequence
  Humanized H1 Heavy Chain Variable Region VH
EVQLVESGGGLVQPGGSLRLSCAASGFTFSDYWMSWVRQAPGKGLEWVAEIRLKSDNYATHYAESVKGRFTISRDNAKNSLYLQMNSLRAEDTAVYYCTRDYYAMEYWGQGTLVTVSS

SEQ ID NO: 4 - protein, 118 aa, Artificial Sequence
  Humanized H2 Heavy Chain Variable Region VH
EVQLVESGGGLVQPGGSLRLSCVASGFTFSDYWMSWVRQAPGKGLEWVAEIRLKSDNYATHYAESVKGRFTISRDDAKNSLYLQMNSLRAEDTAVYYCTRDYYAMEYWGQGTLVTVSS

SEQ ID NO: 5 - protein, 118 aa, Artificial Sequence
  Humanized H37 Heavy Chain Variable Region VH
EVQLVESGGGLVQPGGSLRLSCAASGFTFSDYWMDWVRQAPGKGLEWVGEIRLKSDNYATHYAASVKGRFTISRDDSKNSLYLQMNSLKTEDTAVYYCTRDYYAMEYWGQGTTVTVSS

SEQ ID NO: 6 - protein, 118 aa, Artificial Sequence
  Humanized H38 Heavy Chain Variable Region VH
EVQLLESGGGLVQPGGSLKLSCAVSGFTFSDYWMSWVRQAPGKGLEWVSEIRLKSDNYATHYADSVKGRFTISRDDSKNTLYLQMNSLRAEDTAVYYCTRDYYAMEYWGQGTLVTVSS

SEQ ID NO: 7 - protein, 118 aa, Artificial Sequence
  Humanized H39 Heavy Chain Variable Region VH
EVKLVESGGGLVQPGGSLRLSCAVSGFTFSDYWMDWVRQAPGKGLEWVAEIRLKSDNYATHYAASVKGRFTISRDDSKSRLYLQMNSLKTEDTAVYYCTRDYYAMEYWGQGTTVTVSS

SEQ ID NO: 8 - protein, 112 aa, Artificial Sequence
  Humanized L1 Light Chain Variable Region VL
DIVMTQSPLSLPVTPGEPASISCRSSQTIVHSNGKTYLEWYLQKPGQSPQLLIYKVSNRFSGVPDRFSGSGSGTDFTLKISRVEAEDVGVYYCFQASHVPYTFGQGTKLEIK

SEQ ID NO: 9 - protein, 112 aa, Artificial Sequence
  Humanized L2 Light Chain Variable Region VL
DIVMTQSPLSLPVTPGEPASISCRSSQTIVHSNGKTYLEWYLQKPGQSPQLLIYKVSNRFSGVPDRFSGSGSGTEFTLKISRVEAEDVGVYYCFQASHVPYTFGQGTKLEIK

SEQ ID NO: 10 - protein, 112 aa, Artificial Sequence
  Humanized L37 Light Chain Variable Region VL
DIVMTQTPLSLSVTPGQPASISCKSSQTIVHSNGKTYLYWYLQKPGQSPQLLIYKVSSRFSGVPDRFSGSGSGTDFTLKISRVEAEDVGVYYCFQASHVPYTFGQGTKLEIK

SEQ ID NO: 11 - protein, 112 aa, Artificial Sequence
  Humanized L38 Light Chain Variable Region VL
DIVMTQTPLSLPVTSGEPASISCRSSQTIVHSNGKTYLDWYLQKPGQSPQLLIYKVSNRASGVPDRFSGSGSGTDFTLKISRVEAEDVGVYYCFQASHVPYTFGGGTKVEIK

SEQ ID NO: 12 - protein, 112 aa, Artificial Sequence
  Humanized L39 Light Chain Region VL
DVVMTQTPLSLSVTPGQPASISCKSSQTIVHSNGKTYLEWYLQKPGQSPQLLIYKVSNRFSGVPDRFSGSGSGTDFTLKISRVEAEDVGVYYCFQASHVPYTFGQGTKLEIK

SEQ ID NO: 13 - protein, 5 aa, Artificial Sequence
  H1 VHCDR1 humanized anti-BKB2R antibody
DYWMS

SEQ ID NO: 14 - protein, 19 aa, Artificial Sequence
  H1 VHCDR2 humanized anti-BKB2R antibody
EIRLKSDNYATHYAESVKG

SEQ ID NO: 15 - protein, 7 aa, Artificial Sequence
  H1 VHCDR3 humanized anti-BKB2R antibody
DYYAMEY

SEQ ID NO: 16 - protein, 5 aa, Artificial Sequence
  H2 VHCDR1 humanized anti-BKB2R antibody
DYWMS

SEQ ID NO: 17 - protein, 19 aa, Artificial Sequence
  H2 VHCDR2 humanized anti-BKB2R antibody
EIRLKSDNYATHYAESVKG

SEQ ID NO: 18 - protein, 7 aa, Artificial Sequence
  H2 VHCDR3 humanized anti-BKB2R antibody
DYYAMEY

SEQ ID NO: 19 - protein, 5 aa, Artificial Sequence
  H37 VHCDR1 humanized anti-BKB2R antibody
DYWMD

SEQ ID NO: 20 - protein, 19 aa, Artificial Sequence
  H37 VHCDR2 humanized anti-BKB2R antibody
EIRLKSDNYATHYAASVKG

SEQ ID NO: 21 - protein, 7 aa, Artificial Sequence
  H37 VHCDR3 humanized anti-BKB2R antibody
DYYAMEY

SEQ ID NO: 22 - protein, 5 aa, Artificial Sequence
  H38 VHCDR1 humanized anti-BKB2R antibody
DYWMS

SEQ ID NO: 23 - protein, 19 aa, Artificial Sequence
  H38 VHCDR2 humanized anti-BKB2R antibody
EIRLKSDNYATHYADSVKG

SEQ ID NO: 24 - protein, 7 aa, Artificial Sequence
  H38 VHCDR3 humanized anti-BKB2R antibody
DYYAMEY

SEQ ID NO: 25 - protein, 5 aa, Artificial Sequence
  H39 VHCDR1 humanized anti-BKB2R antibody
DYWMD

SEQ ID NO: 26 - protein, 19 aa, Artificial Sequence
  H39 VHCDR2 humanized anti-BKB2R antibody
EIRLKSDNYATHYAASVKG

SEQ ID NO: 27 - protein, 7 aa, Artificial Sequence
  H39 VHCDR3 humanized anti-BKB2R antibody
DYYAMEY

SEQ ID NO: 28 - protein, 16 aa, Artificial Sequence
  L1 VLCDR1 humanized anti-BKB2R antibody
RSSQTIVHSNGKTYLE

SEQ ID NO: 29 - protein, 7 aa, Artificial Sequence
  L1 VLCDR2 humanized anti-BKB2R antibody
KVSNRFS

SEQ ID NO: 30 - protein, 9 aa, Artificial Sequence
  L1 VLCDR3 humanized anti-BKB2R antibody
FQASHVPYT

SEQ ID NO: 31 - protein, 16 aa, Artificial Sequence
  L2 VLCDR1 humanized anti-BKB2R antibody
RSSQTIVHSNGKTYLE

SEQ ID NO: 32 - protein, 7 aa, Artificial Sequence
  L2 VLCDR2 humanized anti-BKB2R antibody
KVSNRFS

SEQ ID NO: 33 - protein, 9 aa, Artificial Sequence
  L2 VLCDR3 humanized anti-BKB2R antibody
FQASHVPYT

SEQ ID NO: 34 - protein, 16 aa, Artificial Sequence
  L37 VLCDR1 humanized anti-BKB2R antibody
KSSQTIVHSNGKTYLY

SEQ ID NO: 35 - protein, 7 aa, Artificial Sequence
  L37 VLCDR2 humanized anti-BKB2R antibody
KVSSRFS

SEQ ID NO: 36 - protein, 9 aa, Artificial Sequence
  L37 VLCDR3 humanized anti-BKB2R antibody
FQASHVPYT

SEQ ID NO: 37 - protein, 16 aa, Artificial Sequence
  L38 VLCDR1 humanized anti-BKB2R antibody
RSSQTIVHSNGKTYLD

SEQ ID NO: 38 - protein, 7 aa, Artificial Sequence
  L38 VLCDR2 humanized anti-BKB2R antibody
KVSNRAS

SEQ ID NO: 39 - protein, 9 aa, Artificial Sequence
  L38 VLCDR3 humanized anti-BKB2R antibody
FQASHVPYT

SEQ ID NO: 40 - protein, 16 aa, Artificial Sequence
  L39 VLCDR1 humanized anti-BKB2R antibody
KSSQTIVHSNGKTYLE

SEQ ID NO: 41 - protein, 7 aa, Artificial Sequence
  L39 VLCDR2 humanized anti-BKB2R antibody
KVSNRFS

SEQ ID NO: 42 - protein, 9 aa, Artificial Sequence
  L39 VLCDR3 humanized anti-BKB2R antibody
FQASHVPYT

SEQ ID NO: 43 - protein, 5 aa, Mus musculus
  5F12G1 VHCDR1
DYWMS

SEQ ID NO: 44 - protein, 19 aa, Mus musculus
  5F12G1 VHCDR2
EIRLKSDNYATHYAESVKG

SEQ ID NO: 45 - protein, 7 aa, Mus musculus
  5F12G1 VHCDR3
DYYAMEY

SEQ ID NO: 46 - protein, 16 aa, Mus musculus
  5F12G1 VLCDR1
RSSQTIVHSNGKTYLE

SEQ ID NO: 47 - protein, 7 aa, Mus musculus
  5F12G1 VLCDR2
KVSNRFS

SEQ ID NO: 48 - protein, 9 aa, Mus musculus
  5F12G1 VLCDR3
FQASHVPYT

SEQ ID NO: 49 - DNA, 354 bp, Mus musculus
gaagtgaaacttgaggagtctggaggaggcttggtgcaacctggaggatccatgaaactctcctgtgtagtctctggatttactttcagtgactactggatgtcttgggtccgccactctccagagatggggcttgagtgggttgctgaaattagattgaaatctgataattatgcaacacattatgcggagtctgtgaaagggaggttcaccatctcaagagatgattccaaaagtcgtctttatttgcaaataaacagcttaagaggtgaagacactggaatttattactgtacgagggattattatgctatggaatattggggtcaaggaacctcagtcaccgtctcctca

SEQ ID NO: 50 - DNA, 336 bp, Mus musculus
gatgttgtgatgacccaaactccactctccctgcctgtcagtcttggagatcaagcctccatctcttgcagatctagtcagaccattgtacatagtaatggaaaaacctatttagaatggtacctgcagaaaccaggccagtctccaaagctcctgatctacaaagtttccaatcgattttctggggtcccagacaggttcagtggcagtggatcagggacagagttcacactcaagatcagcagagtggagactgaggatctgggagtttattactgctttcaggcttcacatgttccttacacgttcggaggggggaccaagctggaaataaaa

SEQ ID NO: 51 - DNA, 354 bp, Artificial Sequence
  DNA Encoding Humanized H1 Heavy Chain Variable Region
gaggtgcagctggtggagtctgggggaggcttggtccagcctggggggtccctgagactctcctgtgcagcctctggattcacctttagtgactattggatgagctgggtccgccaggctccagggaaggggctggagtgggtggccgaaattagattgaaatctgataattatgcaacacattatgcggagtctgtgaagggccgattcaccatctccagagacaacgccaagaactcactgtatctgcaaatgaacagcctgagagccgaggacacggctgtgtattactgtacgagagattattatgctatggaatattggggccaaggaaccctggtcaccgtctcctca

SEQ ID NO: 52 - DNA, 354 bp, Artificial Sequence
  DNA Encoding Humanized H2 Heavy Chain Variable Region
gaggtgcagctggtggagtctgggggaggcttggtccagcctggggggtccctgagactctcctgtgtagcctctggattcacctttagtgactattggatgagctgggtccgccaggctccagggaaggggctggagtgggtggccgaaattagattgaaatctgataattatgcaacacattatgcggagtctgtgaagggccgattcaccatctccagagacgatgccaagaactcactgtatctgcaaatgaacagcctgagagccgaggacacggctgtgtattactgtacgagagattattatgctatggaatattggggccaaggaaccctggtcaccgtctcctca

SEQ ID NO: 53 - DNA, 354 bp, Artificial Sequence
  DNA Encoding Humanized H37 Heavy Chain Variable Region
gaagttcaactggtcgaaagcggggggggactcgtgcagcccggcggttctcttcgactgtcttgcgccgcttctggtttcacattctctgactattggatggactgggttaggcaagcccctggcaaagggttggaatgggtgggtgagattcgactgaagtctgacaattacgccactcattatgccgccagtgtgaaaggccggtttactatttcccgagatgactccaagaactccctgtacctccaaatgaactctctgaaaactgaggatactgctgtttactattgcactagagactattacgctatggaatattgggggcaaggcactacagtgacagtctctagt

SEQ ID NO: 54 - DNA, 355 bp, Artificial Sequence
  DNA Encoding Humanized H38 Heavy Chain Variable Region
gaggttcaactgttggaaagtggaggggggctcgtacaacctggtggaagcctgaagctcagctgtgctgtgtcaggttttacattctctgactattggatgagctgggttcgtcaagcccctggcaaaggattggagtgggtttccgaaatccgtctcaagtctgacaattacgctactcattacgcagacagcgttaaggggagattcaccatttcacgcgatgattccaaaaacaccctgtatctccagatgaactcactgcgggcagaggacaccgcagtctattactgtaccagggattattatgcaatggagtattggggccagggcacactggtgaccgtaagctccg

SEQ ID NO: 55 - DNA, 354 bp, Artificial Sequence
  DNA Encoding Humanized H39 Heavy Chain Variable Region
gaagtgaaattggtcgaatctggcggagggctcgtccaacccggtggcagcctcagactgtcttgtgcagtatccgggttcacctttagcgattactggatggattgggtccggcaggctcctggcaagggtctggaatgggtagccgagatcagacttaaaagcgacaactacgcaacccactatgccgcaagcgtaaaaggtcgattcaccatatcacgggacgattccaagagccggctgtacttgcagatgaactccttgaaaacagaggacacagccgtgtattattgtactcgggattactacgcaatggaatactggggtcagggaaccacagtaacagtttccagt

SEQ ID NO: 56 - DNA, 336 bp, Artificial Sequence
  DNA Encoding Humanized L1 Light Chain Variable Region
gatattgtgatgactcagtctccactctccctgcccgtcacccctggagagccggcctccatctcctgcaggtctagtcagaccattgtacatagtaatggaaaaacctatttggaatggtacctgcagaagccagggcagtctccacagctcctgatctataaagtttctaatcggttttccggggtccctgacaggttcagtggcagtggatcaggcacagattttacactgaaaatcagcagagtggaggctgaggatgttggggtttattactgctttcaggcttcacatgttccttacacttttggccaggggaccaagctggagatcaaa

SEQ ID NO: 57 - DNA, 336 bp, Artificial Sequence
  DNA Encoding Humanized L2 Light Chain Variable Region
gatattgtgatgactcagtctccactctccctgcccgtcacccctggagagccggcctccatctcctgcaggtctagtcagaccattgtacatagtaatggaaaaacctatttggaatggtacctgcagaagccagggcagtctccacagctcctgatctataaagtttctaatcggttttccggggtccctgacaggttcagtggcagtggatcaggcacagagtttacactgaaaatcagcagagtggaggctgaggatgttggggtttattactgctttcaggcttcacatgttccttacacttttggccaggggaccaagctggagatcaaa

SEQ ID NO: 58 - DNA, 336 bp, Artificial Sequence
  DNA Encoding Humanized L37 Light Chain Variable Region
gatatcgtcatgacccagacccccctgtccctgtctgtaacaccaggccagccagcaagcatctcctgcaagagttcccagaccattgttcatagcaacggcaaaacatacctctactggtatcttcagaagcccggacagtccccccaactgctgatctacaaagtgtcctctcggttctctggagtaccagaccgtttctctggcagtgggagcggcactgattttacactcaagatctcaagggtggaggctgaagacgtcggagtctactactgttttcaggctagtcatgtgccatacaccttcggccagggaaccaagctggaaatcaag

SEQ ID NO: 59 - DNA, 336 bp, Artificial Sequence
  DNA Encoding Humanized L38 Light Chain Variable Region
gatatcgtgatgacacagacacctctctcccttcccgtgacatctggggaaccagcctcaatctcttgcaggtcttcccagacaatcgtccatagcaatggcaaaacatatctggactggtatctccagaagccaggacagagcccacagctgcttatatataaagtctccaatagggcaagtggggtgccagaccggttttccggttcaggttccggcactgactttactcttaagatctccagggtggaggcagaggatgtgggagtgtactattgctttcaggcttcccacgtaccctacacctttggcgggggcactaaggtggagattaag

SEQ ID NO: 60 - DNA, 336 bp, Artificial Sequence
  DNA Encoding Humanized L39 Light Chain Variable Region
gacgtggttatgactcagacccctctctctctctccgtgacacctggccagccagcctcaataagctgtaagtcttcacaaactattgtccacagcaacggaaagacttatttggaatggtacttgcagaaacccggacagtcaccccagctccttatctacaaggtgagcaacagattttccggcgtgcccgatcgcttcagtggatctggctccggcacagattttacactgaaaatttctcgtgtggaggctgaagacgtcggcgtctactactgtttccaggcctcccacgttccctataccttcggccagggaacaaaactggagatcaag

SEQ ID NO: 61 - DNA, 26 bp, Artificial Sequence
  RACE primer
ctcaattttcttgtccaccttggtgc

SEQ ID NO: 62 - DNA, 26 bp, Artificial Sequence
  RACE primer
ctcaagttttttgtccaccgtggtgc

SEQ ID NO: 63 - DNA, 26 bp, Artificial Sequence
  RACE primer
ctcgattctcttgatcaactcagtct

SEQ ID NO: 64 - DNA, 24 bp, Artificial Sequence
  RACE primer
tggaatgggcacatgcagatctct

SEQ ID NO: 65 - DNA, 30 bp, Artificial Sequence
  RACE primer
ctcattcctgttgaagctcttgacaatggg

SEQ ID NO: 66 - DNA, 33 bp, Artificial Sequence
  RACE primer
acactcagcacgggacaaactcttctccacagt

SEQ ID NO: 67 - DNA, 33 bp, Artificial Sequence
  RACE primer
acactctgcaggagacagactcttttccacagt

SEQ ID NO: 68 - DNA, 33 bp, Artificial Sequence
  RACE primer
acactcagcacgggacaaactcttctccacatg

SEQ ID NO: 69 - DNA, 21 bp, Artificial Sequence
  Primer
tcacacaggaaacagctatga

SEQ ID NO: 70 - DNA, 19 bp, Artificial Sequence
  Primer
taatacgactcactatagg

SEQ ID NO: 71 - protein, 364 aa, Homo sapiens
MLNVTLQGPTLNGTFAQSKCPQVEWLGWLNTIQPPFLWVLFVLATLENIFVLSVFCLHKSSCTVAEIYLGNLAAADLILACGLPFWAITISNNFDWLFGETLCRVVNAIISMNLYSSICFLMLVSIDRYLALVKTMSMGRMRGVRWAKLYSLVIWGCTLLLSSPMLVFRTMKEYSDEGHNVTACVISYPSLIWEVFTNMLLNVVGFLLPLSVITFCTMQIMQVLRNNEMQKFKEIQTERRATVLVLVVLLLFIICWLPFQISTFLDTLHRLGILSSCQDERIIDVITQIASFMAYSNSCLNPLVYVIVGKRFRKKSWEVYQGVCQKGGCRSEPIQMENSMGTLRTSISVERQIHKLQDWAGSRQ

SEQ ID NO: 72 - protein, 392 aa, Mus musculus
MPCSWKLLGFLSVHEPMPTAASFGIEMFNVTTQVLGSALNGTLSKDNCPDTEWWSWLNAIQAPFLWVLFLLAALENLFVLSVFFLHKNSCTVAEIYLGNLAAADLILACGLPFWAITIANNFDWVFGEVLCRVVNTMIYMNLYSSICFLMLVSIDRYLALVKTMSMGRMRGVRWAKLYSLVIWGCTLLLSSPMLVFRTMREYSEEGHNVTACVIVYPSRSWEVFTNVLLNLVGFLLPLSVITFCTVRILQVLRNNEMKKFKEVQTERKATVLVLAVLGLFVLCWVPFQISTFLDTLLRLGVLSGCWDEHAVDVITQISSYVAYSNSGLNPLVYVIVGKRFRKKSREVYRVLCQKGGCMGEPVQMENSMGTLRTSISVERQIHKLQDWAGKKQ

SEQ ID NO: 73 - protein, 13 aa, Homo sapiens
KEYSDEGHNVTAC

SEQ ID NO: 74 - protein, 13 aa, Mus musculus
REYSEEGHNVTAC

SEQ ID NO: 75 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 76 - DNA, 981 bp, Homo sapiens
gcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 77 - protein, 107 aa, Homo sapiens [sequence omitted]

SEQ ID NO: 78 - DNA, 324 bp, Homo sapiens
cggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 79 - protein, 326 aa, Homo sapiens; identical to SEQ ID NO: 75

SEQ ID NO: 80 - DNA, 981 bp, Homo sapiens
gctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 81 - protein, 107 aa, Homo sapiens [sequence omitted]

SEQ ID NO: 82 - DNA, 324 bp, Homo sapiens
cggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 83 - protein, 444 aa, Artificial Sequence
  Humanized Hi Heavy Chain + IgG2 constant region
EVQLVESGGGLVQPGGSLRLSCAASGFTFSDYWMSWVRQAPGKGLEWVAEIRLKSDNYATHYAESVKGRFTISRDNAKNSLYLQMNSLRAEDTAVYYCTRDYYAMEYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 84 - protein, 444 aa, Artificial Sequence
  Humanized H2 Heavy Chain + IgG2 constant region
EVQLVESGGGLVQPGGSLRLSCVASGFTFSDYWMSWVRQAPGKGLEWVAEIRLKSDNYATHYAESVKGRFTISRDDAKNSLYLQMNSLRAEDTAVYYCTRDYYAMEYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 85 - protein, 444 aa, Artificial Sequence
  Humanized H37 Heavy Chain + IgG2 constant region
EVQLVESGGGLVQPGGSLRLSCAASGFTFSDYWMDWVRQAPGKGLEWVGEIRLKSDNYATHYAASVKGRFTISRDDSKNSLYLQMNSLKTEDTAVYYCTRDYYAMEYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 86 - protein, 444 aa, Artificial Sequence
  Humanized H38 Heavy Chain + IgG2 constant region
EVQLLESGGGLVQPGGSLKLSCAVSGFTFSDYWMSWVRQAPGKGLEWVSEIRLKSDNYATHYADSVKGRFTISRDDSKNTLYLQMNSLRAEDTAVYYCTRDYYAMEYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 87 - protein, 444 aa, Artificial Sequence
  Humanized H39 Heavy Chain + IgG2 constant region
EVKLVESGGGLVQPGGSLRLSCAVSGFTFSDYWMDWVRQAPGKGLEWVAEIRLKSDNYATHYAASVKGRFTISRDDSKSRLYLQMNSLKTEDTAVYYCTRDYYAMEYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 88 - protein, 219 aa, Artificial Sequence
  Humanized L1 Light Chain + Ig kappa constant region
DIVMTQSPLSLPVTPGEPASISCRSSQTIVHSNGKTYLEWYLQKPGQSPQLLIYKVSNRFSGVPDRFSGSGSGTDFTLKISRVEAEDVGVYYCFQASHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 219 aa, Artificial Sequence
  Humanized L2 Light Chain + Ig kappa constant region
DIVMTQSPLSLPVTPGEPASISCRSSQTIVHSNGKTYLEWYLQKPGQSPQLLIYKVSNRFSGVPDRFSGSGSGTEFTLKISRVEAEDVGVYYCFQASHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 219 aa, Artificial Sequence
  Humanized L37 Light Chain + Ig kappa constant region
DIVMTQTPLSLSVTPGQPASISCKSSQTIVHSNGKTYLYWYLQKPGQSPQLLIYKVSSRFSGVPDRFSGSGSGTDFTLKISRVEAEDVGVYYCFQASHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 219 aa, Artificial Sequence
  Humanized L38 Light Chain + Ig kappa constant region
DIVMTQTPLSLPVTSGEPASISCRSSQTIVHSNGKTYLDWYLQKPGQSPQLLIYKVSNRASGVPDRFSGSGSGTDFTLKISRVEAEDVGVYYCFQASHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 219 aa, Artificial Sequence
  Humanized L39 Light Chain + Ig kappa constant region
DVVMTQTPLSLSVTPGQPASISCKSSQTIVHSNGKTYLEWYLQKPGQSPQLLIYKVSNRFSGVPDRFSGSGSGTDFTLKISRVEAEDVGVYYCFQASHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC